(19) United States
(12) Patent Application Publication
(10) Pub. No.: US 2025/0250641 A1
(43) Pub. Date: Aug. 7, 2025

(54) METHODS AND COMPOSITIONS RELATED TO EPIGENETIC EDITING BY A METHLYLATION-DEPENDENT CAS9

(71) Applicants: Florida State University Research Foundation, Inc., Tallahassee, FL (US); Wageningen Universiteit, Wageningen (NL)

(72) Inventors: Hong Li, Tallahassee, FL (US); Mitchell Roth, Tallahassee, FL (US); Yuerong Shu, Tallahassee, FL (US); Yu Zhao, Tallahassee, FL (US); Renee Hoffman, Tallahassee, FL (US); John van der Oost, Renkum (NL); Despoina Trasanidou, Wageningen (NL)

(21) Appl. No.: 19/046,886

(22) Filed: Feb. 6, 2025

Related U.S. Application Data
(60) Provisional application No. 63/550,225, filed on Feb. 6, 2024.

(51) Int. Cl. C12Q 1/6886; C12N 9/22; C12Q 1/44; C12Q 1/6809; C12Q 1/6827; C12Q 1/686; C12Q 1/6895

(52) U.S. Cl. CPC C12Q 1/6886; C12N 9/22; C12Q 1/44; C12Q 1/6809; C12Q 1/6827; C12Q 1/686; C12Q 1/6895; C12Q 2600/106; C12Q 2600/13; C12Q 2600/154; G01N 2333/922

(57) ABSTRACT

Certain Cas9 molecules are capable of discriminately cleaving nucleic acid based upon its epigenetic pattern. These molecules can be either naturally occurring or engineered. They can be used to diagnose and treat disease.

### CROSS-REFERENCE TO RELATED APPLICATION

This application claims priority to, and the benefit of, U.S. Provisional Patent Application No. 63/550,225, filed Feb. 6, 2024, entitled “METHODS AND COMPOSITIONS RELATED TO EPIGENETIC EDITING BY A METHYLATION-DEPENDENT CAS9,” which is incorporated by reference herein in its entirety.

### STATEMENT REGARDING FEDERALLY SPONSORED RESEARCH

This invention was made with Government Support under Grant No. GM099604 awarded by the National Institutes of Health. The Government has certain right in the invention.

### REFERENCE TO SEQUENCE LISTING

The sequence listing submitted on Feb. 6, 2025, as an .XML file entitled “10850-098WO1_ST26” created on Feb. 5, 2025, and having a file size of 220,135 bytes is hereby incorporated by reference pursuant to 37 C.F.R. § 1.52 (c) (5).

### FIELD

The present disclosure relates Cas9 molecules capable of discriminately cleaving nucleic acids based on epigenetic patterns and methods of using said Cas9 molecules.

### BACKGROUND

CRISPR-Cas9 (Cas9), derived from the CRISPR-mediated immune system in bacteria, has become a powerful genome manipulation tool that allows for precise changes to be made in the DNA of nearly any organism. Directed by a guide RNA (gRNA) that recognizes the target DNA sequence via base pairing, Cas9 proteins catalyze the cleavage of both target DNA strands. Cytosine methylation (5° C.) and its dynamic counterpart, demethylation, are hallmarks of gene regulation in animals and plants, playing pivotal roles in cell differentiation, transposon silencing, aging, pathogenesis of various diseases and development of therapeutics. This important DNA marker is, however, not being recognized by the Cas9 effectors currently in use. To fully harness the power of epigenetic information in genome editing and beyond, ^{5m}C-sensitive Cas9 effectors are highly desired. The discovery of a ^{5m}C-sensitive Cas9 would significantly extend the reach of other Cas9-based tools, including site-specific base-editing, transcription regulation, prime-editing, and virus eradication.

Comprehensive analysis of human cell type methylomes displays well-conserved and cell type-specific DNA methylation patterns in healthy cells. Comparison of the methylation patterns in healthy cells to those in diseased or aging cells unveil disease- and location-specific biomarkers for diagnosis and therapeutic applications. Many cancer types, for instance, are driven by both genetic as well as epigenetic abnormalities. As a result, methylation profiling has been developed as an effective method for disease detection and post-treatment follow-ups. A methylation-sensitive Cas9 would offer a simple enzyme-based utility in discovering and controlling epigenetic changes. It can be programmed to target a single or multiple disease-driven genes that have undergone epigenetic changes. Although Cas9 has been retooled for site-specific methyl addition or removal in cells through fused epigenetic modifiers, these methods do not link DNA methylation to Cas9-mediated recognition or editing. In contrast, methylation-sensitive Cas9 systems can enzymatically respond to methylation changes in cells.

What is needed in the art is a methylation-sensitive Cas9.

### SUMMARY

The present disclosure relates to Cas9 molecules, particularly those which can discriminately cleave based upon methylation status.

In one aspect, disclosed is a method of detecting one or more methylation sites in a specific location on a target nucleic acid, the method comprising: a) exposing a target nucleic acid to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid at a cleavage site differently upon presence of a methylated cytosine residue within the PAM recognition sequence compared to a non-methylated version of the same cytosine residue; and b) detecting whether the Cas9 molecule has cleaved the target nucleic acid, thereby detecting the presence of one or more methylation sites.

In some embodiments, the methylation site within the PAM recognition sequence comprises a cytosine. In some embodiments, the cytosine is a fifth nucleotide within the PAM. In some embodiments, the PAM recognition sequence further comprises a guanine. In some embodiments, the guanine is a sixth nucleotide within the PAM.

In some embodiments, the Cas9 molecule cleaves the target nucleic acid upstream of the PAM sequence. In some embodiments, the Cas9 molecule is ThermoCas9. In some embodiments, the ThermoCas9 is naturally occurring. In some embodiments, the ThermoCas9 comprises SEQ ID NO: 52, or a variation thereof. In some embodiments, the ThermoCas9 is engineered.

In some embodiments, the PAM recognition site further comprises a second cytosine. In some embodiments, the second cytosine is a sixth nucleotide within the PAM. In some embodiments, a site of methylation on the target nucleic acid indicates a higher likelihood of presence of a disease or disorder, including but not limited to cancer or aging, than a non-methylated version of the target nucleic acid.

In some embodiments, more than one methylation site within the target nucleic acid is recognized by the Cas9 molecule. In some embodiments, an epigenetic pattern of the target nucleic acid molecule can be established. In some embodiments, detection occurs by carrying out PCR on non-cleaved target nucleic acid and detecting a product thereof, thereby determining that the target nucleic acid did not comprise one or more methylation sites in a specific location. In some embodiments, detection occurs via a high throughput assay.

Also disclosed is a modified Cas9 molecule, wherein said modified Cas9 molecule cleaves nucleic acid differently upon presence of a methylated cytosine residue within its PAM recognition sequence, wherein said modified Cas9 comprises one or more mutations which alters its PAM recognition site.

Further disclosed is a method of determining an epigenetic pattern in a subject, the method comprising: a) exposing a target nucleic acid from the subject to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid differently upon presence of a methylated cytosine residue within the PAM recognition sequence; and b) detecting whether the Cas9 molecule has cleaved the target nucleic acid at one or more sites, thereby determining the epigenetic pattern.

In some embodiments, the subject is diagnosed with a disease based on the epigenetic pattern. In some embodiments, the subject is determined to have an increased risk of having or developing a disease based on the epigenetic pattern. In some embodiments, a treatment regimen of the subject is determined by the epigenetic pattern. In some embodiments, the subject is already undergoing a treatment for a disease or disorder related to the epigenetic pattern. In some embodiments, success of treatment is evaluated based on results of the epigenetic pattern. In some embodiments, the epigenetic pattern was also evaluated at an earlier timepoint, such that success of treatment can be measured as a function of time. In some embodiments, a risk score is obtained after step b). In some embodiments, the risk score determines how a subject is treated.

A modified Cas9 molecule is disclosed herein, wherein said modified Cas9 molecule cleaves a nucleic acid differently upon presence of a methylated cytosine residue within a PAM recognition sequence of the nucleic acid, wherein said modified Cas9 comprises one or more mutations which alters a PAM recognition site of the modified Cas9.

In some embodiments, the Cas9 molecule is a modified ThermoCas9. In some embodiments, the Cas9 molecule comprises one or more modifications. In some embodiments, the one or more modifications alter a specificity of the Cas9. In some embodiments, the one or more modifications alter an efficiency of the Cas9. In some embodiments, the one or more modifications alter a cleavage rate of the Cas9 molecule. In some embodiments, the one or more modifications alter a recognition rate of the Cas9 molecule. In some embodiments, the one or more modifications alter a cleavage rate of the Cas9 molecule and a recognition rate of the Cas9 molecule.

Also disclosed is a method of treating a disease or disorder in a subject in need thereof, wherein the disease or disorder is associated with a loss of methylation at one or more locations in a target nucleic acid of the subject, wherein the method comprises: exposing the target nucleic acid to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid at an unmethylated cleavage site, and further wherein the unmethylated cleavage site comprises a cytosine residue within the PAM recognition sequence; and administering a therapeutic agent to the subject, wherein the therapeutic agent prevents the loss of methylation at the one or more locations in the target nucleic acid.

In some aspects, disclosed herein is a method of treating a crop plant for improved yield, wherein the crop plant comprises a loss of methylation at one or more locations in a target nucleic acid, the method comprising exposing the target nucleic acid to a Cas9 molecule and administering a therapeutic agent to the crop plant, wherein the target nucleic acid molecule comprises a protospacer adjacent motif (PAM) recognition sequence that is recognized by the Cas9 molecule, wherein the Cas9 molecule cleaves the target nucleic acid at an unmethylated cleavage site, wherein the unmethylated cleavage site comprises a cytosine residue within the PAM recognition sequence, and wherein the therapeutic agent alters one or more nucleotide sequences at the targeted nucleic acid relative to an untreated control.

In some embodiments, the disease or disorder comprises cancer. In some embodiments, the disease or disorder comprises aging. In some embodiments, the loss of methylation causes arrested or delayed development or a risk of pesticide intoxication. In some embodiments, the Cas9 molecule is ThermoCas9. In some embodiments, the Cas9 molecule is a modified ThermoCas9. In some embodiments, the Cas9 molecule comprises one or more modifications. In some embodiments, the one or more modifications alter a specificity of the Cas9. In some embodiments, the one or more modifications alter an efficiency of the Cas9. In some embodiments, the one or more modifications alter a cleavage rate. In some embodiments, the one or more modifications alter a recognition rate of the Cas9 molecule. In some embodiments, the one or more modifications alter a cleavage rate of the Cas9 molecule and a recognition rate of the Cas9 molecule.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIGS. 1A, 1B, and 1C show that the ThermoCas9 activity is sensitive to DNA methylation. FIG. 1A shows the schematic Cas9 activity (left) targeting methylated or unmethylated DNA plasmids. DNA cleavage (right) results on methylated or unmethylated DNA plasmids. NTS, nontarget strand; TS, target strand; ^{5m}CpG_{w}, full methylation on both nontarget and target strand cytosine; M.SssI methyltransferase methylates CpG sequence; HaeIII methyltransferase methylates GGCC sequence; HaeIII restriction enzyme cleaves unmethylated GGCC sequence. For DNA cutting reactions, ThermoCas9 at 1 mM was incubated with 100 nM DNA at 37° C. for 30 minutes. Cleavage results are analyzed on agarose gels. FIG. 1B shows the schematic Cas9 activity (left) targeting methylated or unmethylated DNA oligo substrates. DNA cleavage (right) results on methylated or unmethylated DNA oligo substrates. ^{5m}CpGrs, oligo substrate with methylation on target strand cytosine; ^{5m}CpG_{NTS}, oligo substrate with methylation on nontarget strand cytosine; ^{5m}CpG_{w}, oligo substrate with methylation on both nontarget and target strand cytosine. For DNA cutting reactions, ThermoCas9 at 1 mM was incubated with 100 nM DNA oligo substrate at 37° C. for 60 minutes. Cleavage results are analyzed on polyacrylamide gels and imaged by fluorescent probes associated with each strand (HEX-NTS and FAM-TS). “*” indicates the possible non-denatured HEX-NTS-target DNA. FIG. 1C shows the competition assay results. Cleavage of a pUC19 plasmid containing a protospacer followed by a 5′-NNNNCGAA-3′ (SEQ ID NO: 7) PAM by ThermoCas9 in presence of no or increasing concentrations of unmethylated or various methylated DNA oligos of the same protospacer. Left shows the representative gel images. Right shows the fraction of cleavage as a function of competitor DNA concentrations in log scales. Three experimental replications were performed.

FIGS. 2A, 2B, 2C, and 2D show the visualization of epigenetic profiles and methylation-dependent screening results. FIG. 2A shows the differential methylation profiles for 10 selected cell lines in ENCODE on chromosome 17. Colored lines indicate the degree of methylation from green indicating fully unmethylated to red indicating fully methylated CpG. The number associated with each cell line indicates the percent of CpG differentially methylated from those in HEK293 cells. FIG. 2B shows the close-up view of the methylation profiles for selected cells of the targeted PRDX4 and EMX1 sites, respectively. FIG. 2C shows the bisulfite sequencing results for the targeted EMX1 (T4), VEGFA (T3) and PRDX4 (T5) sites, respectively. The protospacer and PAM sequences are shown under the sequencing traces. Filled circles indicate methylated cytosine. “(C)” indicates unmethylated cytosine that was converted to thymine in bisulfite sequencing. “(G)” also indicate unmethylated cytosine when sequencing the complementary DNA strand. “C*” indicates possible hemi-methylated sites or incomplete bisulfite treatment. FIG. 2D shows the ICE indel frequencies for the four targeted editing sites. Error bars are calculated from the indel percent of three biological replications. Filled circles under the bars indicate methylated sites. Open circles under the bars indicates unmethylated sites.

FIGS. 3A, 3B, 3C, 3D, 3E, 3F, and 3G show the overview of ThermoCas9 CryoEM structures. FIG. 3A shows the domain architecture and sequence features of ThermoCas9 with domain boundaries indicated. FIG. 3B shows the secondary structures of the observed R-loop structure and nucleotide labeling. “PK” denotes pseudoknot. Dash lines indicate unmodeled regions. FIG. 3C shows the cartoon representation of the observed R-loop structure of the post-cleavage conformation. “PK” denotes pseudoknot. Dash lines indicate unmodeled regions. FIG. 3D shows the CryoEM density of the pre-cleavage conformation of ThermoCas9 color coded as in panel a. FIG. 3E shows the cartoon representation of ThermoCas9 of the pre-cleavage conformation color coded as in panel a. FIG. 3F shows the CryoEM density of the post-cleavage conformation of ThermoCas9 color coded as in panel a. FIG. 3G shows the cartoon representation of ThermoCas9 of the post-cleavage conformation color coded as in panel a.

FIGS. 4A, 4B, 4C, and 4D show the structural transitions in active sites of ThermoCas9. FIG. 4A shows the cartoon representation of the pre-cleavage (OPEN) (top) and the post-cleavage (CLOSED) (bottom) conformation highlighting the two active sites and DNA. The HNH active site residues are colored in green and those for RuvC are in blue. Magnesium ions are shown as orange spheres. FIG. 4B shows the close-up views of two cleavage sites. Top, aligned sequences of select Cas9 in regions that comprise the two nuclease centers, HNH and RuvC, respectively. Residues participate in metal coordination and additional catalytic functions are highlighted by different colors. Bottom, cryoEM density and stick models of two active sites are shown around the cleaved nucleotides, the magnesium ion and the coordinated water molecules and protein residues. Dash lines indicate close contact distances. Scissile phosphate oxygen atoms are labeled as “O_{Sp}” for pro-Sp, “O_{Rp}” for pro-Rp and “O_{NUC}” for the nucleophilic oxygen. Water molecules are labels as W1, W2, etc. and the protein residues are labeled.

FIG. 4C shows the superimposed RuvC active structures between the pre-cleavage (tan) and post-cleavage (blue) conformation. Magnesium ions are shown as orange spheres. Black dash lines indicate the contacts that undergo changes from the pre- to post-cleavage transition. FIG. 4D shows the bacterial cell survival assay results of the wild-type (WT), Lys711 to alanine (K711A) and Asp723 to alanine (D723A) of ThermoCas9. The survival rate is calculated by dividing the colony forming unit (CFU) on the arabinose-plus (inducing ccdB toxic protein) plate by that on the arabinose-minus plate.

FIGS. 5A, 5B, and 5C show the PAM recognition by ThermoCas9. FIG. 5A shows the cartoon representations of ThermoCas9 structure highlighting PAM recognition region. Inset shows stick models of PAM nucleotides and the protein residues within 3.5 Å of PAM. FIG. 5B shows the recognition of each PAM base pair in stick models overlaid with cryoEM density. Dash lines indicate close contacts between the protein residues and the DNA bases. FIG. 5C shows the cell survival assay results of wild-type (WT), Asp1017 to alanine (D1017A) and Ser1019 to alanine (S1019A) of ThermoCas9. The survival rate is calculated by dividing the colony forming units (CFU) on the arabinose-plus (inducing ccdB toxic protein) plate by that on the arabinose-minus plate.

FIGS. 6A, 6B, and 6C show the specimen preparation and DNA binding analysis results. FIG. 6A shows the elution profile of ThermoCas9, following Ni-NTA affinity purification on a Heparin column. The shaded fractions are analyzed on an SDS-PAGE gel and pooled before used in biochemical analysis or cryoEM sample preparation. FIG. 6B shows the elution profile of ThermoCas9, following incubation with the in vitro transcribed single guide RNA at 37° C. for 30 minutes, on a S200i gel filtration column. The shaded fractions were analyzed on an SDS-PAGE gel and were used immediately for cryoEM analysis. FIG. 6C shows the DNA binding competition assays with ThermoCas9. Gel images illustrate the cleavage results of a DNA plasmid by ThermoCas9 in the presence of increasing amount of four different competing double-stranded DNA oligos: no methylation (CpG), methylated on the non-target strand (^{5m}CpG_{NTS}), methylated on the target strand (₅ₘCpG_{TS}), and methylated on both strands (^{5m}CpG_{w}), respectively.

FIG. 7 shows the flow cytometry plots displaying green fluorescent protein (GFP) fluorescence intensity (x-axis) versus red fluorescent protein (RFP) fluorescence intensity (y-axis; to control for auto-fluorescence) following 78 h post transfection for the ThermoCas9 in HEK293 cells. GFP+ cells fall within the Q4 gate. The flow cytometry plots for non-transfected cells are shown on the top row. Experiments were conducted in triplicate.

FIG. 8 shows the flow cytometry plots displaying green fluorescent protein (GFP) fluorescence intensity (x-axis) versus red fluorescent protein (RFP) fluorescence intensity (y-axis; to control for auto-fluorescence) following 78 h post transfection for the ThermoCas9 in HCT116 cells. GFP+ cells fall within the Q4 gate. The flow cytometry plots for non-transfected cells are shown on the top row of FIG. 7. Experiments were conducted in triplicate.

FIGS. 9A, 9B, 9C, and 9D show the analysis of indel frequencies within HEK293 cells transformed with ThermoCas9 using Sanger sequencing and Inference of CRISPR Edits (ICE) tool. The purple box indicates the protospacer sequence and the orange box indicates the PAM sequence. T #R #denotes “T” for “Target” and “R” for “Replicate”.

FIGS. 10A, 10B, 10C, 10D, and 10E show the analysis of indel frequencies within HCT116 cells transformed with ThermoCas9 using Sanger sequencing and Inference of CRISPR Edits (ICE) tool. The purple box indicates the protospacer sequence and the orange box indicates the PAM sequence. T #R #denotes “T” for “Target” and “R” for “Replicate”.

FIGS. 11A, 11B, 11C, 11D, and 11E show the Cryo-EM image collection, analysis and 3D reconstruction results of the ThermoCas9 bound with a cognate DNA substrate. FIG. 11A shows the example micrograph and 2D class averages (scale bar 50 nm). FIG. 11B shows the data collection, particle selection, and reconstruction flowchart. All classes reconstructed and fitted with atomic models are indicated with reported resolutions. FIG. 11C shows the final map (upper) used for building the model of the post-cleavage and dsDNA-bound state (CLOSED) and analyzed by Resmap. Resolutions are color-coded according to a scale bar, showing the comparably high-resolution inner core. The Fourier Shell Correlation (FSC) curves of the refined model (lower). 0.143 FSC cutoff was used for resolution estimation. FIG. 11D shows the final map (upper) used for building the model of the post-cleavage and target strand-bound state (CLOSED) and analyzed by Resmap. Resolutions are color-coded according to a scale bar, showing the comparably high-resolution inner core. The Fourier Shell Correlation (FSC) curves of the refined model (lower). 0.143 FSC cutoff was used for resolution estimation. FIG. 11E shows the final map (upper) used for building the model of the pre-cleavage state (OPEN) and analyzed by Resmap. Resolutions are color-coded according to a scale bar, showing the comparably high-resolution inner core. The Fourier Shell Correlation (FSC) curves of the refined model (lower). 0.143 FSC cutoff was used for resolution estimation.

FIG. 12 shows the Cryo-EM density maps for ThermoCas9 observed at the post-cleavage (CLOSED) and pre-cleavage (OPEN) states shown for the protein-(top) and nucleic acids-(middle) in two orientations. Each functional domain or nucleic acid element is labeled. The bottom row shows close-up views of the density around the target strand cleavage site of the CLOSED and OPEN states, respectively.

FIGS. 13A and 13B show the detailed interactions between ThermoCas9 and the single guide RNA. FIG. 13A shows the observed percent of five charged amino acids involved in contacting guide RNA for ThermoCas9 and four other Cas9 variants. The coordinates used for analysis are 8DLK for AccCas9, 6JDJ for Nme1Cas9, and 7S4X for SpyCas9. FIG. 13B shows the depiction of the secondary structure of the single guide RNA and the contacts with ThermoCas9 for the CLOSED state.

FIGS. 14A, 14B, and 14C show the Cryo-EM image collection, analysis and 3D reconstruction results of the ThermoCas9 bound with a methylated DNA substrate. FIG. 14A shows an example micrograph and 2D class averages (scale bar 50 nm). FIG. 14B shows the data collection, particle selection, and reconstruction flowchart. The only class containing closed conformation is with target strand only. Due to redundance, no model was refined to the final stage.

FIGS. 15A, 15B, 15C, and 15D shows the Cryo-EM image collection, analysis and 3D reconstruction results of the AceCas9 bound with a methylated DNA substrate. FIG. 15A shows the data collection, particle selection, and reconstruction flowchart. All classes reconstructed and fitted with atomic models are indicated with reported resolutions. FIG. 15B shows an example micrograph and 2D class averages (scale bar 100 nm). FIG. 15C shows the Fourier Shell Correlation (FSC) curves of the three refined models. 0.143 FSC cutoff was used for resolution estimation. FIG. 15D shows the final map used for building the model of the uncleaved state 1 and analyzed by Resmap. Resolutions are color-coded according to a scale bar, showing the comparably high-resolution inner core.

FIGS. 16A, 16B, and 16C show the structural features of AceCas9 bound with a methylated DNA. FIG. 16A shows the schematic of AceCas9 protein and the nucleic acids used in cryoEM structure study. FIG. 16B shows the structure of AceCas9 bound with its guide RNA (sgRNA) and methylated DNA. Top, cryoEM density with each domain and nucleic acids color coded as in panel a and labeled. Bottom, cartoon representation of AceCas9 bound with the methylated DNA color coded as in panel a and labeled. Insets show close-up views of the target strand cleavage site and the PAM interactions. “Me” indicates the methyl group on the methylated cytosine. Dash lines indicate close contacts between the DNA bases and protein residues. FIG. 16C shows the stick models (left) showing the contacts of the two phosphate lock residues, Glu839 and Glu840, with the kinked target strand. Arrows indicate the Glu839 to arginine and Glu840 to tyrosine or leucine (RY or RL), respectively. The gel analysis of phosphate lock residue mutants RY and RL cleaving unmethylated and methylated DNA plasmid (right). Methylated DNA was obtained with treatment with HaeIII methyltransferase. HaeIII restriction endonuclease (HaeIII endo) is included as a control.

FIG. 17 shows when DNA is methylated, it is not cleaved by Cas9 at the same rate as non-methylated DNA.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In aspects of the invention the terms “chimeric RNA”, “chimeric guide RNA”, “guide RNA”, “single guide RNA” and “synthetic guide RNA” are used interchangeably and refer to the polynucleotide sequence comprising the guide sequence, the tracr sequence and the tracr mate sequence. The term “guide sequence” refers to the sequence within the guide RNA that specifies the target site and may be used interchangeably with the terms “guide” or “spacer”. The term “tracr mate sequence” may also be used interchangeably with the term “direct repeat(s)”.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, cd. (1987)).

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerivisae include pYepSecl (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (In Vitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd cd., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Banciji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (Sec, Groenen et al., Mol. Microbiol., 10:1057-1065 [1993]; Hoc et al., Emerg. Infect. Dis., 5:254-263 [1999]; Mascpohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

The term “disorder”, as used herein, is to be understood in the broadest sense. The term denotes: (i) any type of medical condition, that is, any morphological and/or physiological alterations in the target samples (i.e. the cells and/or tissues) exhibiting characteristics of a dysfunctional and/or aberrant cellular phenotype a as compared to unaffected (wild-type) control samples; and/or (ii) any morphological, physiological and/or pharmacological difference between the respective target and reference samples. Examples of alterations according to (i) may relate inter alia to cell size and shape (enlargement or reduction), cell proliferation (increase in cell number), cell differentiation (change in physiological state), apoptosis (programmed cell death) or cell survival. Examples of differences according to (ii) includes inter alia tumor samples vs. healthy controls (for the purpose of diagnosis or recurrence monitoring), aggressive vs. non-aggressive tumor samples (i.e. different tumor stages and/or tumor sub-types; for the purpose of prognostic analyses), conditions relating to treatment regimens such as responsiveness vs. non-responsiveness to a particular therapy for a given disorder/medical condition. Thus, the term disorder may be interpreted as a any kind of difference between two or more samples based on which said samples may be distinguished and/or classified.

In a method according to the present invention, the disorder is a cancer, that is, a type of malignant neoplasm (also referred to as carcinoma) including inter alia colon, lung, liver, breast, ovary, and pancreas cancer, melanoma, neuronal tumors (e.g., gliobastoma, astrocytoma, medullobastoma), and the like.

The term “having a predisposition to develop a disorder”, as used herein, denotes any cellular phenotype being indicative for a pre-state of a disorder, i.e. an intermediate state in the transformation of a normal into an aberrant phenotype. In other words, the term denotes a state of risk of developing a disorder.

The term “identifying one or more candidate genes/loci”, as used herein, should be interpreted in the sense of “selecting” at least one candidate gene from the group of genes present in a given sample that undergo differential methylation. The term “candidate genes” (herein also referred to as “candidate loci”), as used herein, relates to any genetic loci comprising in their nucleic acid sequence one or more nucleic acid sites that may be present in a methylated state and in an unmethylated state. Within the context of the present invention, the term gene is not necessarily restricted to sequences (open reading frames) encoding a protein but includes intergenic regions as well. The selection (i.e. the number and/or type of candidate genes/loci chosen) may vary, for example, depending on treatment modalities of a disease or disorder to be analyzed, including therapeutic intervention, diagnostic criteria such as disease stages, and disease monitoring and surveillance for the disease in the subject to be treated, from whom the sample to be analyzed is derived. Additionally, the term “identifying” encompasses the determination of the extent of the differential DNA methylation in the at least one target sample and the at least one reference sample and comparing the results obtained.

The one or more candidate genes/loci identified may be subjected to further analysis individually or they may be clustered to one or more candidate gene/loci signatures, wherein the entities of each signature are analyzed en bloc (i.e. together). The term “candidate gene/loci signatures”, as used herein, denotes subsets of at least two candidate genes/loci that are related to each other, for example, encoding functionally equivalent proteins or proteins participating in the same signaling pathway, or the like.

The term “DNA methylation”, as used herein, denotes the type of chemical modification of DNA that involves the addition of a methyl group to DNA, for example to the C5 carbon atom of the cytosine pyrimidine ring or to the N6 nitrogen atom of the adenosine purine ring, with the first option being particularly preferred herein. This modification can be inherited and subsequently removed without changing the original DNA sequence. As such, it is part of the epigenetic code and the most well characterized epigenetic mechanism.

The term “differential DNA methylation”, as used herein, denotes a condition in which a particular candidate gene is (at one or more nucleic acid sites comprised in its sequence) methylated in the at least one target sample but unmethylated in the at least reference sample, or vice versa, in which a particular candidate gene is (at one or more nucleic acid sites comprised in its sequence) unmethylated in the at least one reference sample but methylated in the at least target sample.

The term “CpG dinucleotide sites” (or “CpG sites), as used herein, refers to regions of DNA where a cytosine nucleotide is located immediately adjacent to a guanine nucleotide in the linear sequence. “CpG” stands for cytosine and guanine separated by a phosphate (i.e., —C-phosphate-G-). The “CpG” notation is used to distinguish a cytosine followed by guanine from a cytosine base paired to a guanine. There are regions of the DNA that have a higher concentration of CpG sites, known as CpG islands. Many genes in mammalian genomes have CpG islands associated with the transcriptional start site (including the promoter) of the gene.

Hypermethylation (i.e. an increased level of methylation) of CpG sites within the promoters of genes can lead to their silencing, a feature found, e.g., in a number of human cancers (for example the silencing of tumor suppressor genes). In contrast, the hypomethylation (i.e. a reduced level of methylation) of CpG sites has been associated with the over-expression of oncogenes within cancer cells (reviewed, e.g., in Robertson, K. D. and Wolffe, A. P. (2000), supra; Li, E. (2002), supra; Bird, A. P. (2002), supra; Klose, R. J. and Bird, A. P. (2006) Trends Biochem. Sci. 31, 89-97).

### General Description

### Cas9 Molecules

While CRISPR-Cas9 has transformed genome editing, it has yet to fully leverage the pervasive presence of DNA methylation. To fill this gap, disclosed herein are biochemical, structural, and human cell characterizations of an epigenetic-specific Cas9, such as ThermoCas9. ThermoCas9 efficiently binds and cleaves DNA upstream of its protospacer adjacent motif (PAM) 5′-NNNNCG-3′ (SEQ ID NO: 1) or 5′-NNNNCC-3′ (SEQ ID NO: 2), as shown in Example 1. Methylation of the first cytosine in either PAM sequence (^{5m}CpG or ^{5m}CpC), however, significantly inhibits ThermoCas9 activity. Methylation-sensitive editing by ThermoCas9 is demonstrated in two human cell lines that differ in methylation landscape (Example 1). Cryogenic electron microscopy structures of ThermoCas9 in pre- and post-cleavage states at 2.8 Å and 2.2 Å resolution, respectively, reveal the molecular basis for the stringent requirement of the unmethylated cytosine in its function and provide guidance for further enzyme engineering. The methylation-dependent activity of ThermoCas9 opens doors to epigenetic genome screening and the associated technologies, as discussed herein.

The II-C Cas9 protein Geobacillus thermodenitrificans T12 Cas9 (ThermoCas9) is controlled by cytosine-containing PAM sequences^{25, 26}. ThermoCas9 has a 5′-NNNNCNR-3′ (R=purine) PAM²⁶ (SEQ ID NO: 3) and exhibits efficient editing activities in human cells 27. Herein, it is shown to be similarly sensitive to ^{5m}C at its 5^{th} position. ThermoCas9 therefore provides an opportunity for sensing both ^{5m}CpC and ^{5m}CpG. Whereas ^{5m}CpC methylation has increasingly been shown to occur in stem and brain cells 28-30 and on the mitochondrial genome 31, a large majority of methylations occur on the CpG sequence as ^{5m}CpG^{32, 33}. Therefore, a Cas9 sensitive to SmCpG permits more broad epigenetic applications. The sensitivity of ThermoCas9 to ^{5m}CpG and ^{5m}CpC both in vitro and in vivo are shown herein (Example 1). Also demonstrated is a proof-of-concept application of ThermoCas9 in performing genome editing in a DNA methylation-dependent manner. CryoEM structures of the active ThermoCas9 bound with DNA substrates at two functional states, are also reported. Structural and biochemical characterization reveal the molecular mechanism for ThermoCas9 in discriminating against methylated DNA substrates.

Based on these findings, disclosed herein is a method of detecting one or more methylation sites in a specific location on a target nucleic acid, the method comprising: a) exposing a target nucleic acid to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid at a cleavage site differently upon presence of a methylated cytosine residue within the PAM recognition sequence compared to a non-methylated version of the same cytosine residue; and b) detecting whether the Cas9 molecule has cleaved the target nucleic acid, thereby detecting the presence of one or more methylation sites.

As discussed above, the methylation site is within the PAM recognition sequence. This methylation site can comprise, for example, a cytosine. In the example of naturally occurring ThermoCas9, this cytosine can be at the 5^{th} position within the PAM sequence (5′-NNNNCG-3′ (SEQ ID NO: 1)). This position can be followed by either another cytosine at position 6 (5′-NNNNCC-3′ (SEQ ID NO: 2)), or can be followed by a guanine at position 6, as in SEQ ID NO: 1. In one example, the PAM sequence can comprise one or more downstream purines, which can further enhance the activity of Cas9. An example of that can be found in SEQ ID NOS: 4-8. It is noted, however, that the Cas9 (or its corresponding CRISPR molecule, such as gRNA) can be modified so that the PAM recognition sequence is different than naturally occurring PAM, and can therefor cleave a different target as well. A Cas9 molecule typically cleaves within 2-6 nucleotides of the PAM sequence. This is typically upstream of the PAM recognition sequence.

In some embodiments, the methylation site within the PAM recognition sequence comprises a cytosine. In some embodiments, the cytosine is a fifth nucleotide within the PAM. In some embodiments, the PAM recognition sequence further comprises a guanine. In some embodiments, the guanine is a sixth nucleotide within the PAM. In some embodiments, the PAM recognition site further comprises a second cytosine. In some embodiments, the second cytosine is a sixth nucleotide within the PAM. In some embodiments, a site of methylation on the target nucleic acid indicates a higher likelihood of presence of a disease or disorder, including but not limited to cancer or aging, than a non-methylated version of the target nucleic acid.

The target nucleic acid sequence may be cleaved by the Cas protein, and optionally the cleavage can be DNA cleavage. The target nucleic acid strand comprising the target sequence may be double stranded DNA and this can result in a double stranded break in the target nucleic acid and its complement. It is stated herein that the Cas9 molecule cleaves the target nucleic acid at a cleavage site differently upon presence of a methylated cytosine residue within the PAM recognition sequence compared to a non-methylated version of the same cytosine residue. What is meant by “differently” is that the Cas9 can cleave the unmethylated version of the same sequence with a different efficiency or ability than the methylated version. For example, the methylated version may be cleaved less often, or may be cleaved less quickly, than the methylated version. By “less often” is meant that the methylated version may be cleaved 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% less often than the unmethylated version. It can also be cleaved less quickly. For example, the cleavage rate (time to cleavage) of the methylated version may be increased by 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% or more, for example.

In some embodiments, more than one methylation site within the target nucleic acid is recognized by the Cas9 molecule. In some embodiments, an epigenetic pattern of the target nucleic acid molecule can be established. In some embodiments, detection occurs by carrying out PCR on non-cleaved target nucleic acid and detecting a product thereof, thereby determining that the target nucleic acid did not comprise one or more methylation sites in a specific location. In some embodiments, detection occurs via a high throughput assay.

### Engineered Cas9

The Cas9 molecule disclosed herein, which is capable of discriminating between methylated and unmethylated nucleic acid, can be naturally occurring or can be engineered. When the Cas9 is naturally occurring, it can be, for example, ThermoCas9, which is represented by SEQ ID NO: 52. ThermoCas9 can be mutated, however, in a variety of ways which increases its usefulness. This can be done to increase the efficiency, processivity, or specificity, for example, This can include the conditions under which Cas9 cleaves the target nucleic acid, or the rate at which cleavage occurs.

A modified Cas9 molecule is disclosed herein, wherein said modified Cas9 molecule cleaves a nucleic acid differently upon presence of a methylated cytosine residue within a PAM recognition sequence of the nucleic acid, wherein said modified Cas9 comprises one or more mutations which alters a PAM recognition site of the modified Cas9.

In some embodiments, the Cas9 molecule is a modified ThermoCas9. In some embodiments, the Cas9 molecule comprises one or more modifications. In some embodiments, the one or more modifications alter a specificity of the Cas9. In some embodiments, the one or more modifications alter an efficiency of the Cas9. In some embodiments, the one or more modifications alter a cleavage rate of the Cas9 molecule. In some embodiments, the one or more modifications alter a recognition rate of the Cas9 molecule. In some embodiments, the one or more modifications alter a cleavage rate of the Cas9 molecule and a recognition rate of the Cas9 molecule.

In some embodiments, the Cas9 molecule cleaves the target nucleic acid upstream of the PAM sequence. In some embodiments, the Cas9 molecule is ThermoCas9. In some embodiments, the ThermoCas9 is naturally occurring. In some embodiments, the ThermoCas9 comprises SEQ ID NO: 52, or a variation thereof. In some embodiments, the ThermoCas9 is engineered.

For example, amino acid residues may be substituted conservatively or non-conservatively. Conservative amino acid substitutions refer to those where amino acid residues are substituted for other amino acid residues with similar chemical properties (e.g., charge or hydrophobicity) and therefore do not alter the functional properties of the resulting polypeptide. Similarly it will be appreciated by a person of average skill in the art that nucleic acid sequences may be substituted conservatively or non-conservatively without affecting the function of the polypeptide. Conservatively modified nucleic acids are those substituted for nucleic acids which encode identical or functionally identical variants of the amino acid sequences. It will be appreciated by the skilled reader that each codon in a nucleic acid (except AUG and UGG; typically the only codons for methionine or tryptophan, respectively) can be modified to yield a functionally identical molecule. Accordingly, each silent variation (i.e. synonymous codon) of a polynucleotide or polypeptide, which encodes a polypeptide of the present invention, is implicit in each described polypeptide sequence.

Cas9 molecules include engineered Cas9 polypeptides (engineered, as used in this context, means merely that the Cas9 molecule or Cas9 polypeptide differs from a reference sequences, and implies no process or origin limitation). An engineered Cas9 polypeptide can comprise altered enzymatic properties, e.g., altered nuclease activity (as compared with a naturally occurring or other reference Cas9 molecule) or altered helicase activity. An engineered Cas9 molecule or Cas9 polypeptide can have nickase activity (as opposed to double strand nuclease activity). In certain embodiments, an engineered Cas9 molecule or Cas9 polypeptide can have an alteration that alters its size, e.g., a deletion of amino acid sequence that reduces its size, e.g., without significant effect on one or more Cas9 activities. In certain embodiments, an engineered Cas9 molecule or Cas9 polypeptide can comprise an alteration that affects PAM recognition, e.g., an engineered Cas9 molecule can be altered to recognize a PAM sequence other than that recognized by the endogenous wild-type PI domain. In certain embodiments, a Cas9 molecule or Cas9 polypeptide can differ in sequence from a naturally occurring Cas9 molecule but not have significant alteration in one or more Cas9 activities.

When the ThermoCas9 is modified, it can be modified to increase a variety of desirable characteristics or to minimize a variety of undesirable characteristics. For example, the Cas9 can be modified so that it differs in specificity, processivity, hybridization conditions, or how or where cleavage and/or recognition occur.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In an embodiment, a Cas9 molecule or Cas9 polypeptide can comprises one or more mutations or differences, e.g., at least 1, 2, 3, 4, 5, 10, 15, 20, 30, 40, or 50 mutations relative to a reference, e.g., a parental, Cas9 molecule, such as ThermoCas9 (SEQ ID NO: 52).

In certain embodiments, a mutation or mutations do not have a substantial effect on a Cas9 activity, e.g., a Cas9 activity described herein. In other embodiments, a mutation or mutations have a substantial effect on a Cas9 activity, e.g., a Cas9 activity described herein.

### Epigenetic Analysis

Further disclosed is a method of determining an epigenetic pattern in a subject, the method comprising: a) exposing a target nucleic acid from the subject to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid differently upon presence of a methylated cytosine residue within the PAM recognition sequence; and b) detecting whether the Cas9 molecule has cleaved the target nucleic acid at one or more sites, thereby determining the epigenetic pattern.

In some embodiments, a biomarker (or an epigenetic marker) is methylated or unmethylated in a normal sample (e.g., normal or control tissue without disease, or normal or control body fluid, stool, blood, serum, and amniotic fluid), most importantly in healthy stool, blood, serum, amniotic fluid or other body fluid. In other embodiments, a biomarker (or an epigenetic marker) is hypomethylated or hypermethylated in a sample from a patient having or at risk of an increased risk of disease; for example, at a decreased or increased (respectively) methylation frequency of at least about 50%, at least about 60%, at least about 70%, at least about 75%, at least about 80%, at least about 85%, at least about 90%, at least about 95%, or about 100% in comparison to a normal sample. In one embodiment, a sample is also hypomethylated or hypermethylated in comparison to a previously obtained sample analysis of the same patient having or at risk of an increased risk of a disease or disorder.

In some embodiments, the subject is diagnosed with a disease based on the epigenetic pattern. In some embodiments, the subject is determined to have an increased risk of having or developing a disease based on the epigenetic pattern. In some embodiments, a treatment regimen of the subject is determined by the epigenetic pattern. In some embodiments, the subject is already undergoing a treatment for a disease or disorder related to the epigenetic pattern. In some embodiments, success of treatment is evaluated based on results of the epigenetic pattern. In some embodiments, the epigenetic pattern was also evaluated at an earlier timepoint, such that success of treatment can be measured as a function of time. In some embodiments, a risk score is obtained after step b). In some embodiments, the risk score determines how a subject is treated.

In some embodiments, DNA (e.g., genomic DNA such as extracted genomic DNA or treated genomic DNA) is isolated by any means standard in the art, including the use of commercially available kits. Briefly, wherein the DNA of interest is encapsulated in by a cellular membrane the biological sample is disrupted and lysed by enzymatic, chemical or mechanical means. In some cases, the DNA solution is then cleared of proteins and other contaminants e.g. by digestion with proteinase K. The DNA is then recovered from the solution. In such cases, this is carried out by means of a variety of methods including salting out, organic extraction or binding of the DNA to a solid phase support. In some instances, the choice of method is affected by several factors including time, expense and required quantity of DNA.

Wherein the sample DNA is not enclosed in a membrane (e.g. circulating DNA from a cell free sample such as blood or urine) methods standard in the art for the isolation and/or purification of DNA are optionally employed (See, for example, Bettegowda et al. Detection of Circulating Tumor DNA in Early- and Late-Stage Human Malignancies. Sci. Transl. Med, 6 (224): ra24. 2014). Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. In some cases, the person skilled in the art also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, and charged switched surfaces. In some instances, once the nucleic acids have been extracted, methylation analysis is carried out using the Cas9 molecules described herein.

Changes in DNA methylation leading to aberrant gene silencing have been demonstrated in several human cancers (reviewed, e.g., in Robertson, K. D. and Wolffe, A. P. (2000) Nat. Rev. Genet. 1, 11-19). Hypermethylation of promoters was demonstrated to be a frequent mechanism leading to the inactivation of tumor suppressor genes (Bird, A. P. (2002) Genes Dev. 16, 6-21). DNA methylation can lead to the silencing of genes by means of two distinct mechanisms: first, methylation at CpG dinucleotide sites that prevents the binding of transcription factors with their cognate DNA recognition sequences; and second, recognition of methyl-CpG dinucleotide sites by a family of methyl-CpG binding proteins (MBD), thus eliciting repression of methylated DNA.

Within the context of the present disclosure, a candidate gene/locus may comprise only a single nucleic acid site that is differentially methylated between the at least one target sample and the at least one reference sample. However, it may also be possible that a particular candidate gene/locus. In a scenario of more than one differentially methylated nucleic acid site comprised in a particular candidate gene/locus the subsequent analysis of the methylation pattern may be performed separately for each individual nucleic acid site or for the candidate gene in its entirety. Accordingly, the terms “candidate gene/locus” and “nucleic acid” may be used interchangeably herein depending on the type of analysis performed. Thus, the method of the present invention may comprise the selection and analysis of one or more candidate genes, one or more nucleic acid sites or a combination thereof.

In analogy, the DNA methylation state (or level) may refer to an individual nucleic acid site or to the overall methylation level of a candidate gene/locus comprising more than one nucleic acid sites. When the plurality of nucleic acid sites comprises entities of different types a candidate gene/locus is considered to be in the “methylated” state if in the at least one target sample as compared to the at least one reference sample a higher number of unmethylated nucleic acid sites becomes methylated than vice versa. One the other hand, a candidate gene/locus is considered to be in the “unmethylated” state if in the at least one target sample as compared to the at least one reference sample a higher number of methylated nucleic acid sites becomes unmethylated than vice versa.

The methylation state can indicate that a subject has a disease, or has a higher likelihood of developing that disease or disorder. For example, the methylation pattern can indicate that a subject has a predisposition to develop a disorder.

### Detection/Diagnosing/Treating

Detection of cleavage products can be done by a variety of ways known to those of skill in the art. For example, Kaminski et al. (Kaminski, M. M., Abudayych, O. O., Gootenberg, J. S. et al. CRISPR-based diagnostics. Nat Biomed Eng 5, 643-656 (2021), herein incorporated by reference in its entirety, discloses a variety of methods for detection a Cas9 cleavage product. Examples include, but are not limited to, PCR amplification, sequencing, lateral flow assays, and high throughput assay means. The method of claim 1, wherein detection occurs via high throughput assay.

Disclosed herein is a method of determining epigenetic pattern in a subject with a disease or disorder, the method comprising: a) exposing a target nucleic acid from the subject to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid differently upon presence of a methylated cytosine residue within the PAM recognition sequence; and b) detecting whether the Cas9 molecule has cleaved the target nucleic acid at one or more sites, thereby determining epigenetic pattern. The subject can be diagnosed with a disease based on the epigenetic pattern, as discussed above.

The sample obtained from the subject may be a blood sample, a fine needle aspirate (FNA) sample, a tissue sample, a fecal sample or any combination thereof. The sample may comprise cell-free DNA. The sample may comprise a small sample volume, for example, from about 1 nanogram to about 15 ng. The sample may comprise a small sample volume, for example from about 1 cell to about 1000 cells; from about 1 cell to about 500 cells; from about 1 cell to about 100 cells. A sample may comprise a first portion comprising a blood sample and a second portion comprising a tissue sample or a fecal sample.

A result of assaying may be compared to a result obtained from a control sample. The control sample may comprise a database of control samples. The control sample may comprise at least: 1, 2, 3, 4, 5, 6, 7, 8, 9, 10, 15, 20, 30, 40, 50, 60, 70, 80, 90, 100, 150, 200 independent samples. The control sample may comprise at least 5 independent samples. The control sample may comprise at least 10 independent samples. The control sample may comprise at least 5 independent samples. The control sample may comprise at least 20 independent samples. The control sample may comprise at least 50 independent samples. The control sample may comprise at least 100 independent samples. The control sample may comprise a blood sample, an FNA sample, a tissue sample, or any combination thereof. The control sample may be obtained from a healthy volunteer. The control sample may be obtained from a subject having received a positive diagnosis of a disease such as cancer. The control sample may be obtained from a subject having a specific cancer type, such as a colorectal cancer, a colon cancer, etc. The control sample may include a sample previously obtained from the same subject, such as a sample obtained at an early point in time. The control sample may include a sample obtained from a different subject.

An epigenetic biomarker may be a metabolic-related biomarker, an immune-related biomarker, or any combination thereof. A comparison of the result to a result from a control sample may identify the sample as benign or malignant for a cancer. A result may include assaying a sample for a population of immune cells, including a number of immune cells or immune cell subtypes. Immune cell subtypes may include T cells, B cells, neutrophils, basophils, eosinophils, or any combination thereof. A result may include assaying a sample for a population of immune cells and quantifying one or more markers expressed by the population of immune cells.

A method may comprise identifying a presence or an absence of an early stage cancer or a late stage cancer in a sample. The cancer may be colorectal cancer, a colon cancer, or others. The method may identify the sample as having a particular stage of cancer, such as stage I, II, III, or IV. The method may identify the sample as having an aggressive type of cancer. The identification may be based on a comparison to a control sample. For example, the sample may be assayed for a result and the result may be compared to a result obtained from a control sample. The control sample may comprise samples obtained from early-stage cancer and late stage cancer, aggressive types of cancer, stage I cancers, stage II cancers, stage III cancers, stage IV cancers, metastatic cancers, or any combination thereof. The assaying may include assaying for at least a portion of a biomarker. The comparison may include comparing a presence or an absence of an epigenetic modification between the control sample and the sample. The comparison may include comparing a differential gene expression, a presence or an absence of a sequence variant, a copy number, a presence or an absence of an epigenetic modification, a patient's genetic history, a patient's environmental history, or any combination thereof.

A method may identify the sample as representative of a subtype of the cancer, such as an aggressive type of cancer. A method may identify the sample as representative of a subtype of the cancer, such as a tissue type (i.e. colorectal cancer). A method may identify the sample as representative of a subtype of the cancer, such as a stage I, stage II, stage III, or stage IV cancer. A method may identify the sample as representative of a subtype of the cancer, such as a colon cancer that may be a serrated adenoma or a tubular adenoma. A method may identify the sample as representative of a subtype of the cancer, such as a colon cancer that may be CMS1, CMS2, CMS3, or CMS4.

A result obtained from assaying may be input into a computer processor. A result obtained from assaying may be input into a trained algorithm. A result including the presence or absence of an epigenetic modification may be input into the trained algorithm. This can be used to calculate a risk score. A trained algorithm may be a classifier, a supervised machine learning algorithm, or a molecular classifier. Epigenetic data (or additionally gene expression data, sequence variant data, copy number data, immune population data, or others) may in some cases be improved through the application of algorithms designed to normalize and or improve the reliability of the data. Data analysis may employ a computer or other device, machine or apparatus for application of the various algorithms described herein due to the large number of individual data points that may be processed. A “machine learning algorithm” may refer to a computational-based prediction methodology, also known to persons skilled in the art as a “classifier”, employed for characterizing epigenetic data, gene expression data, sequence variant data, copy number data, any combination thereof or others. The data obtained from a sample may be input to the algorithm in order to classify the sample, such as benign or malignant for a cancer. Supervised learning generally involves “training” a classifier with a training set to recognize the distinctions among classes or disease states and then “testing” the accuracy of the classifier on an independent test set. For new, unknown samples the classifier can be used to predict the class in which the samples belong, such as benign or malignant for a cancer.

A trained algorithm may identify significant differences in epigenetic data, such as a significant difference in a presence or an absence of an epigenetic modification, as determined by feature selection using LIMMA (linear models for micro array data) and SVM (support vector machine) for classification of malignant vs. benign samples. Rank or weight denotes the marker significance (lower rank, higher significance) after Benjamini and Hochberg correction for False Discovery Rate (FDR). A trained algorithm may include a support vector machine (SVM) algorithm, a random forest algorithm, or a combination thereof.

LIMMA may be used for feature selection. Classification may be performed with a random forest algorithm or SVM methods. Markers that repeatedly appear in multiple iterative rounds of training, classification, and cross validation may be identified and ranked. A joint set of core features may be created using the top ranked features. Biomarkers with a non-zero repeatability score may be selected as significant.

A result of a trained algorithm may be output in a report. Results may be presented as a report on a computer screen or as a paper record. In some cases, the report may include, but is not limited to, such information as one or more of the following: the number of biomarkers comprising an epigenetic modification, a classification of a sample as benign or malignant for a cancer, the suitability of the original sample, a diagnosis, a statistical confidence for the diagnosis, the likelihood of cancer or malignancy, a recommendation for further treatment, or any combination thereof.

The comparison to a control sample may be performed by a trained algorithm. A trained algorithm may be trained to identify feature selections within a data set. A trained algorithm may classify a sample as benign or malignant for a cancer. A cancer may include a colorectal cancer, or a colon cancer.

In some cases, the methods may include identifying a sample as benign or malignant for cancer. In some cases, the method may include identifying a sample as premalignant or precancerous. In some cases, the methods may include identifying a presence of or likelihood of developing a tumor, neoplasm, or cancer. A cancer may include colon cancer, colon cancer, a rectal cancer, or any combination thereof. In some cases, the methods may include identifying a presence of a premalignant condition or a precancerous lesion or growth. A premalignant condition or precancerous lesion or growth may comprise a polyp (such as an adenomatous polyp), a nonpolyp, an adenoma, a dysplasia (such as high grade or low grade), or any combination thereof. In some cases, the methods may include distinguishing a premalignant condition from a benign condition (such as a benign polyp, benign lesion, benign hyperplastic tissue, benign hyperplasia, or the like).

The methods may include comparing a result obtained from assaying a sample to a result obtained from a control or derivative thereof. The comparing may identify the sample as a precancerous lesion or precancerous growth. The comparing may distinguish a precancerous lesion or growth from a benign condition. The comparing may be performed by a trained algorithm. A precancerous lesion or growth may be identified by performing the methods as described herein on a blood sample. The sample may comprise cell-free DNA.

Assaying a sample may be performed in the absence of a screening procedure. The methods herein may provide a replacement or alternative to a screening procedure. A screening procedure may include a colonoscopy, an assay performed on a stool sample provided by the subject, a sigmoidoscopy, or any combination thereof. A benefit of the method may include an alternative pre-screening tool that does not require a colonoscopy or providing a stool sample. The method may provide a result having greater than 90% sensitivity and greater than 80% specificity to distinguish a precancerous lesion or growth from a benign condition. When a subject receives a result identifying the sample as benign, the method may permit a subject to opt out or not receive a screening procedure.

A method may provide a result in the absence of a further medical procedure such as a result that may include an identification of the sample as a malignant or benign for a cancer. A further medical procedure may include: obtaining a second sample from the subject, such as an invasive sample (such as a biopsy) or a blood sample; performing an imaging scan on a portion of the subject; performing surgery on the subject; or a combination thereof.

A method may include repeating the assaying. A method may include repeating the comparing to a control sample, such as comparing to a different control sample. A method may provide a result that includes a recommendation for monitoring a change over time in the result. A method may include assaying a second sample from the subject. The second sample may be obtained from the subject at a different period of time, such as an earlier period of time or a later period of time. A method may provide a result that includes a recommendation for the subject to receive a surgery.

A trained algorithm may be trained with a training set of samples. A trained algorithm may be validated with a validation set of samples. The validation set of samples may be independent of the training set. An independent sample may be input into the trained algorithm that may be independent of both the training set and the validation set.

Also disclosed is a method of treating a disease or disorder in a subject in need thereof, wherein the disease or disorder is associated with a loss of methylation at one or more locations in a target nucleic acid of the subject, wherein the method comprises: exposing the target nucleic acid to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid at a cleavage site when that site is not methylated, and further wherein the cleavage site comprises a cytosine residue within the PAM recognition sequence; and administering a treatment to the subject, wherein the treatment prevents the loss of methylation at the one or more locations in the target nucleic acid. In some embodiments, the disease or disorder comprises cancer. In some embodiments, the disease or disorder comprises aging. Such diseases and disorders include, but are not limited to, those found in Jin Z, Liu Y. DNA methylation in human diseases. Genes Dis. 2018 Jan. 31; 5 (1):1-8, herein incorporated by reference in its entirety.

In some embodiments, the Cas9 molecule is ThermoCas9. In some embodiments, the Cas9 molecule is a modified ThermoCas9. In some embodiments, the Cas9 molecule comprises one or more modifications. In some embodiments, the one or more modifications alter a specificity of the Cas9. In some embodiments, the one or more modifications alter an efficiency of the Cas9. In some embodiments, the one or more modifications alter a cleavage rate. In some embodiments, the one or more modifications alter a recognition rate of the Cas9 molecule. In some embodiments, the one or more modifications alter a cleavage rate of the Cas9 molecule and a recognition rate of the Cas9 molecule.

### Crop Plant Epigenomics

Epigenetics has emerged as an important research field for crop improvement. Heritable epigenetic changes can arise independently of DNA sequence alterations and have been associated with altered gene expression and transmitted phenotypic variation. By modulating plant development and physiological responses to environmental conditions, epigenetic diversity-naturally, genetically, chemically, or environmentally induced- can help optimize crop traits. Presently, there are difficulties in transferring the knowledge of the epigenetic mechanisms from model plants to crops. The present disclosure provides methods of improving, treating, and/or optimizing a crop plant using the ThermoCas9 of any preceding aspect. As used herein, a “crop plant” refers to a plant that is grown for a specific purpose including but not limited to food source, fiber, and/or fuel, wherein said crop plants can be grown and harvested for profit and/or sustenance. “Crop plants” include, but are not limited to food crops (grown for human consumption), feed crops (grown for livestock consumption), fiber crops (grown for the production of textiles, such as for example cotton, hemp, and jute), oil crops (grown for consumption or industrial uses, such as for example corn, canola, and soybeans), Ornamental crops (grown for landscaping and gardening), and/or industrial crops (grown for various personal and industrial uses, such as for example tobacco and rubber).

In some aspects, disclosed herein is a method of treating a crop plant for improved yield, disease resistance, nutritional contents, and/or other desirable traits, wherein the crop plant comprises a loss of methylation at one or more locations in a target nucleic acid, the method comprising exposing the target nucleic acid to a Cas9 molecule and administering a therapeutic agent to the crop plant, wherein the target nucleic acid molecule comprises a protospacer adjacent motif (PAM) recognition sequence that is recognized by the Cas9 molecule, wherein the Cas9 molecule cleaves the target nucleic acid at an unmethylated cleavage site, wherein the unmethylated cleavage site comprises a cytosine residue within the PAM recognition sequence, and wherein the therapeutic agent alters one or more nucleotide sequences at the targeted nucleic acid relative to an untreated control. As used herein, “altering one or more nucleotide sequences” refers to nucleotide changes known in the art, including but not limited to insertions and deletions (INDELs), non-homologous end joining (NHEJ) repair, and HDR repair (combining a cleavage event with introducing one or more genes or fragments thereof). In some embodiments, the therapeutic agent alters the one or more nucleotide sequences by adding, deleting, and/or exchanging one or more nucleotides within the nucleotide sequence.

In some embodiments, the loss of methylation causes arrested or delayed development or a risk of pesticide intoxication. In some embodiments, the Cas9 molecule is ThermoCas9. In some embodiments, the Cas9 molecule is a modified ThermoCas9. In some embodiments, the Cas9 molecule comprises one or more modifications. In some embodiments, the one or more modifications alter a specificity of the Cas9. In some embodiments, the one or more modifications alter an efficiency of the Cas9. In some embodiments, the one or more modifications alter a cleavage rate. In some embodiments, the one or more modifications alter a recognition rate of the Cas9 molecule. In some embodiments, the one or more modifications alter a cleavage rate of the Cas9 molecule and a recognition rate of the Cas9 molecule.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Molecular Basis for Epigenetic Editing by a Methylation-Dependent Cas9

### Results

### ThermoCas9 Discriminates Against 5″ CpC and 5m CpG Sequence

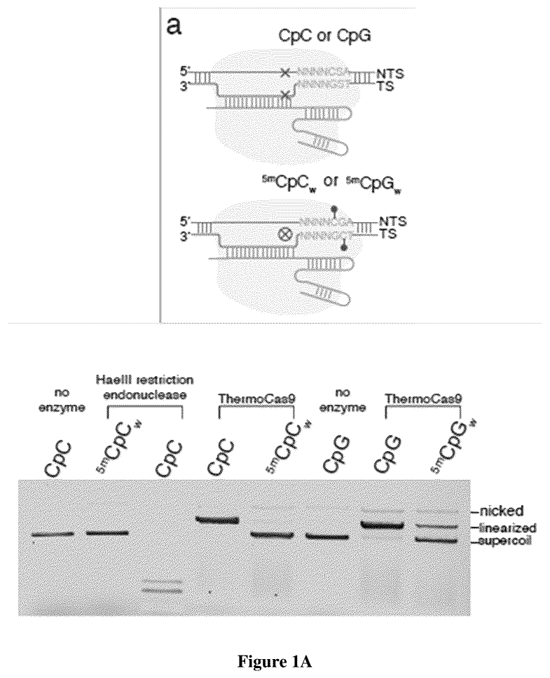

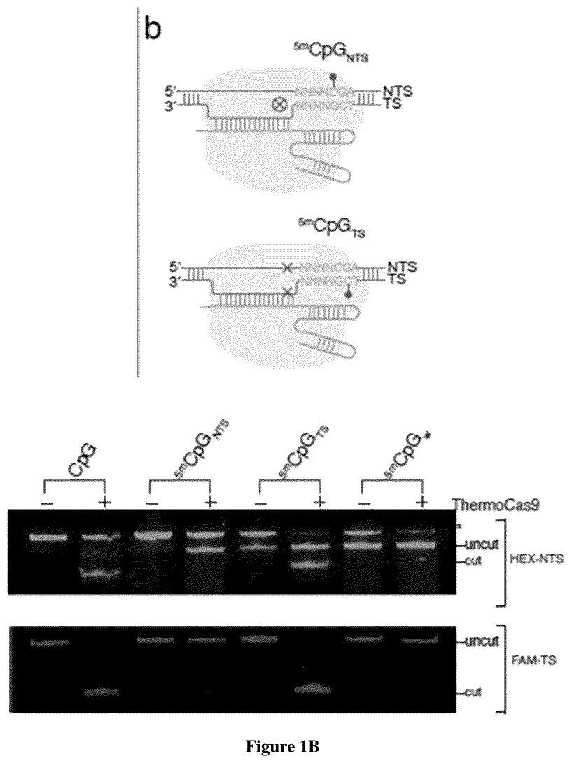

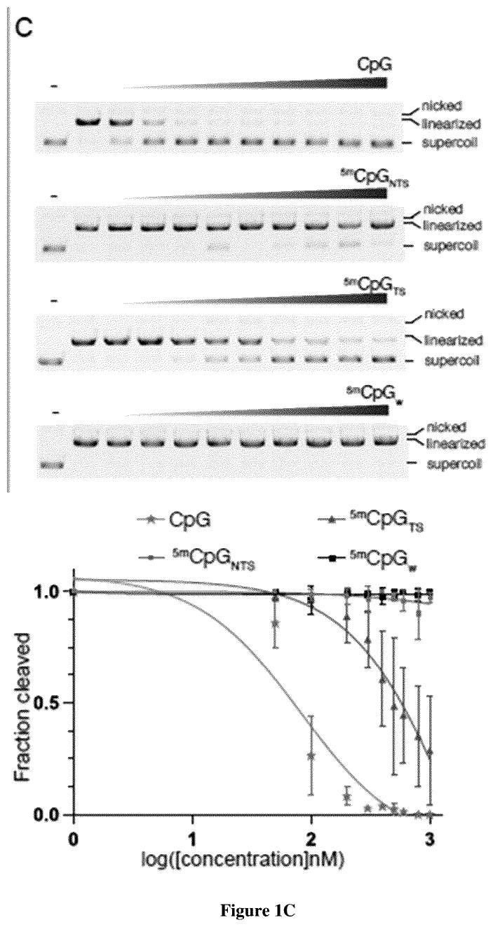

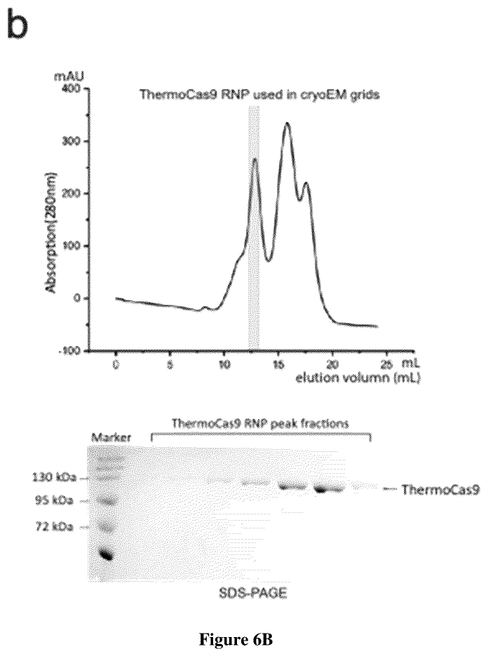

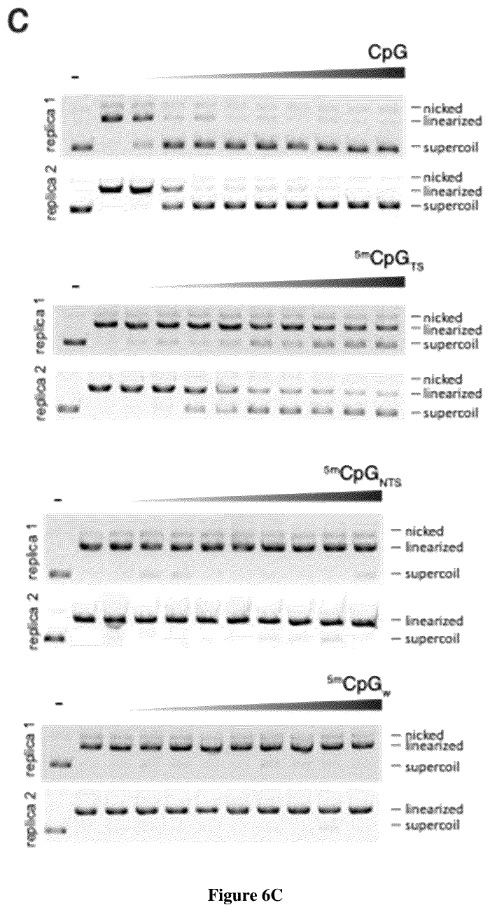

It has been previously demonstrated that ThermoCas9 exhibits a broad PAM specificity, with only the 5^{th} position strictly requiring a C: G pair while downstream purines further enhance the activity (optimal PAM, 5′-NNNNCCAA, SEQ ID NO: 4). To test if ThermoCas9 is sensitive to methylation of the cytosine on the 5th position in its PAM, a single guide RNA (sgRNA) was programmed to target in vitro a 23 base pair (bp) DNA sequence adjacent to either a CpC-(5′-NNGGCCA-3′ SEQ ID NO: 5) or a CpG-containing PAM (5′-NNNNCGA-3′ SEQ ID NO: 6). Methylation (i) was introduced on the 5′-NNGGCCA-3′ PAM (SEQ ID NO: 5) by the HaeIII methyltransferase (recognition site 5′-GGCC-3′) to 5′-NNGG^{5m}CCA-3′ (SEQ ID NO: 5), and (ii) on the 5′-NNNNCGA-3′ PAM (SEQ ID NO: 6) by the M.SssI methyltransferase (recognition site 5′-CG-3′) to 5′-NNNN^{5m}CGA-3′ (SEQ ID NO: 6). Remarkably, while ThermoCas9 cleaved both unmethylated DNA substrates efficiently, it had diminished activity against the DNA associated with either PAM sequence containing ^{5m}C (FIG. 1A & FIG. 6). To distinguish the effect of ^{5m}C on the nontarget from that on the target strand, synthetic oligo DNA duplexes containing strand-specific methylation were used, and it showed that ThermoCas9 is more sensitive to methylation on the nontarget than on the target strand. Methylation on both strands caused the strongest inhibition (FIG. 1B & FIG. 6).

It was shown that inhibition of AceCas9 by DNA methylation occurs at a functional step after AceCas9 binding to DNA. AceCas9 maintains a good binding affinity for methylated DNA, however, it does not cut it. Thus, the present disclosure examines if ThermoCas9 is subjected to a similar mechanism of inhibition. A competition assay was employed in which DNA duplexes formed from either methylated or unmethylated synthetic oligos were added to DNA cleavage reactions to allow them to compete with the nonmethylated plasmid substrates (FIG. 1C & FIG. 6). While unmethylated DNA duplex inhibited plasmid cleavage efficiently, methylated DNA duplex failed to inhibit the reaction even at concentrations as high as 1 mM (FIG. 1C & FIG. 6). Consistent with the strand-specific methylation sensitivity shown in the oligo cleavage assay (FIG. 1B), the DNA oligos containing methylation either on the nontarget strand alone or on both strands had the weakest competition (FIG. 1C & FIG. 6). This result shows that, unlike AceCas9, ThermoCas9 has significantly reduced affinity for the DNA containing a single methyl group at the cytosine at the 5th position of the PAM.

### Methylation-Dependent Genome Editing in Human Cells by ThermoCas9

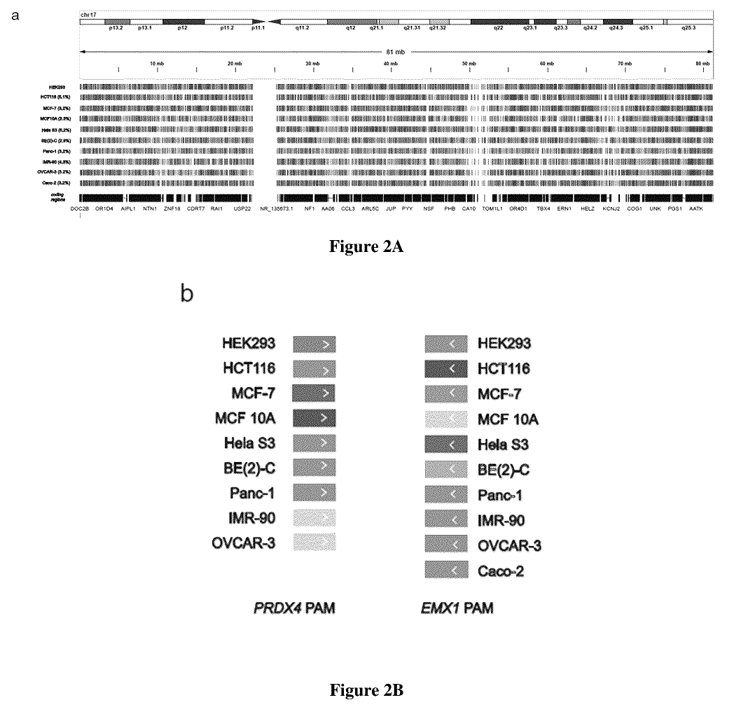

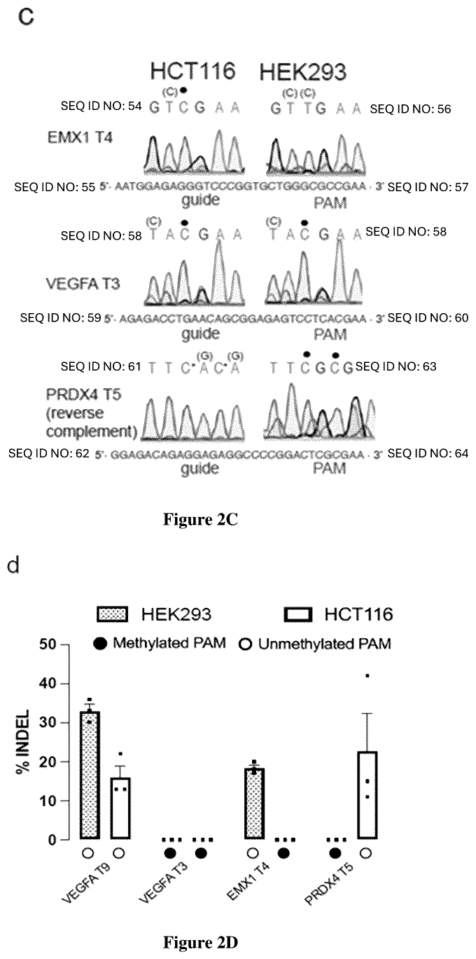

Human genomes undergo dynamic methylation changes in an environment- and disease-dependent manner. The reprogrammability and methylation sensitivity of ThermoCas9 offer the possibility of differential gene editing in human cells that differ in methylation landscape. To quantify the divergence of methylation profiles across various human cells, in silico analysis of reduced-representation bisulfite sequencing (RRBS) data for ten cell lines in the ENCODE database that are derived from various healthy and disease tissues (FIG. 2A). Their methylation status was compared against that of control HEK293 cells. Notably, approximately 3-6% of CpG sequences (>30,000 CpG sites) in the former cells exhibited opposing methylation status to their counterparts in HEK293 cells under a stringent selection criterion (i.e., completely methylated vs. completely unmethylated). The differentially methylated CpG sites are distributed across gene promoters, coding and non-coding regions, including those encoding disease-associated proteins (FIG. 2A). This observation shows tissue- and disease-specific gene editing strategies guided by CpG methylation profiles.

The utility of ThermoCas9 was tested in methylation-dependent editing in cells that differ in methylation profile. Using the in silico analysis results, three DNA targeting sites in the context of the PAM 5′-NNNNCGAA-3′ (SEQ ID NO: 7) for ThermoCas9 that show different methylation between the HEK293 and the HCT116 cells (FIG. 2B). A protospacer (target 4, T4) on the EMX1 gene associated with a PAM that is methylated in HCT116 while not methylated in HEK293 cells was selected. A protospacer on the PRDX4 gene (target 5, T5) was used which is associated with a PAM that is methylated in HEK293 but not in HCT116 cells. The protospacer sequence chosen from the VEGFA gene (target 3, T3) does not show methylation data in ENCODE for either HEK293 or HCT116 cells but was also included for analysis. In addition, another protospacer of the VEGFA gene (target 9, T9) associated with the 5′-NNNNCCAA-3′ (SEQ ID NO: 8) was selected as a control site because it is unmethylated in both cells. In addition, T9 was previously shown to be efficiently edited in HEK293 cells by ThermoCas9. RRBS of the T3, T4, and T5 sites was performed on genomic DNA isolated from the experimental HEK293 and HCT116 cells, respectively, to confirm their reported methylation profile. T4 and T5 showed methylation patterns consistent with those identified in ENCODE whereas T3, surprisingly, exhibited methylation in both cell types (FIG. 2C).

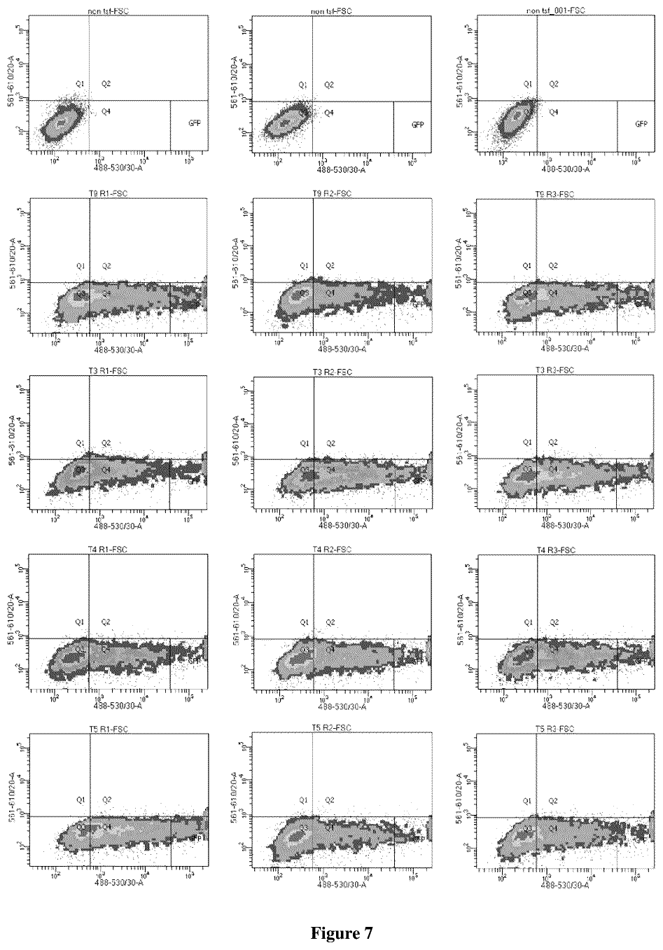

Endogenous genome editing experiments were then performed in HEK293 and HCT116 cells using ThermoCas9 programmed to target T3, T4, T5, and T9, respectively. The average insertions and/or deletions (indels) of each target site were quantified by the indel frequencies calculated using the interference of CRISPR edits from Sanger sequencing trace data (ICE, Synthego) (FIG. 2D & FIGS. 7-10). As expected, ThermoCas9 successfully edited the unmethylated VEGFA T9 site with mean indel frequencies up to 33% in HEK293 and 16% in HCT116, respectively. On the contrary, ThermoCas9 was unable to edit T3 of which the PAM is methylated in both cell lines, resulting in a null mean indel frequency for both HEK293 and HCT116 cells. At the two differentially methylated sites, T4 and T5, in the two cell lines, methylation-dependent editing was observed. As expected, ThermoCas9 was capable of editing the unmethylated EMX1 T4 in HEK293 with the mean indel frequencies reaching 18.3%, but not the same site in HCT116 cells where it is methylated, resulting in a mean null indel frequency. Similarly at the PRDX4 T5 site, ThermoCas9 did not edit the methylated T5 in HEK293 cells, leading to a mean null indel frequency, while it resulted in a mean indel frequency of 22.6% in HCT116 cells where the PAM was unmethylated (FIG. 2D & FIGS. 7-10). These data show that ThermoCas9 can be repurposed for methylation-dependent editing, which is consistent with the in vitro cleavage and binding results.

### Structural Basis for Activation of ThermoCas9

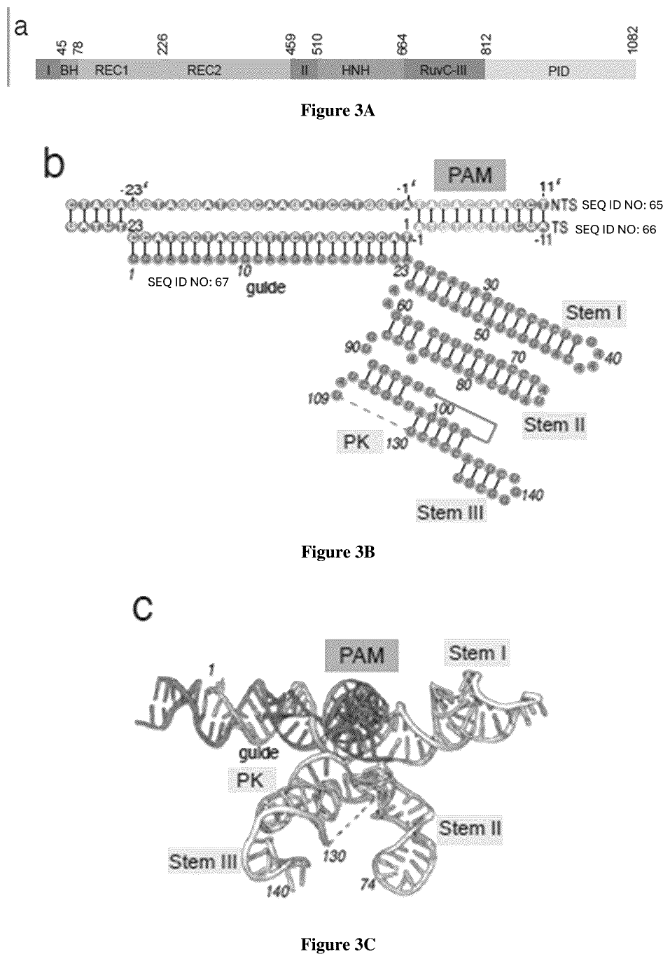

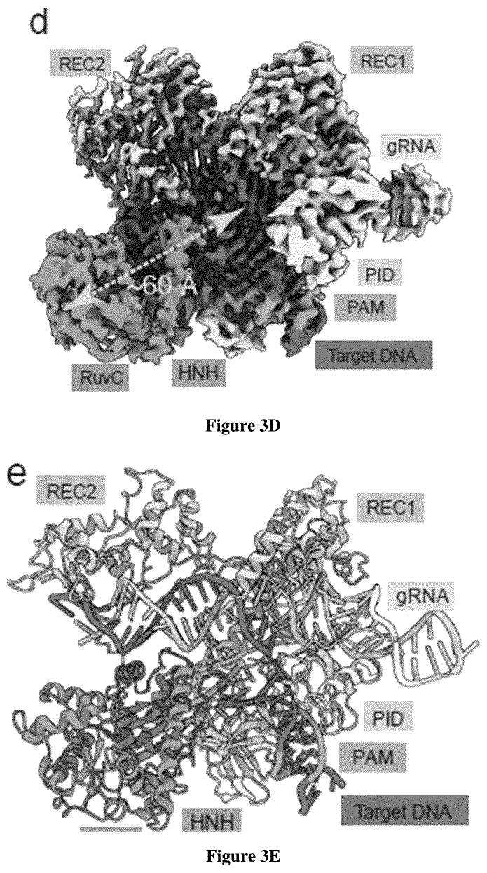

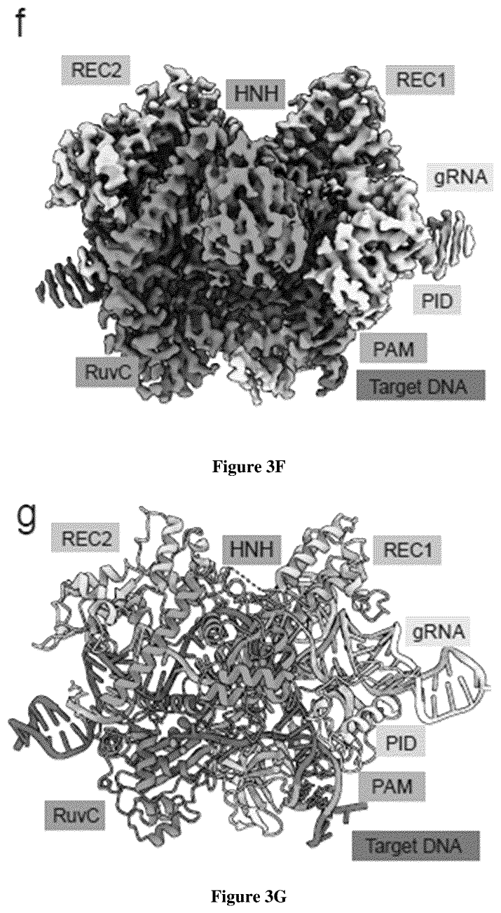

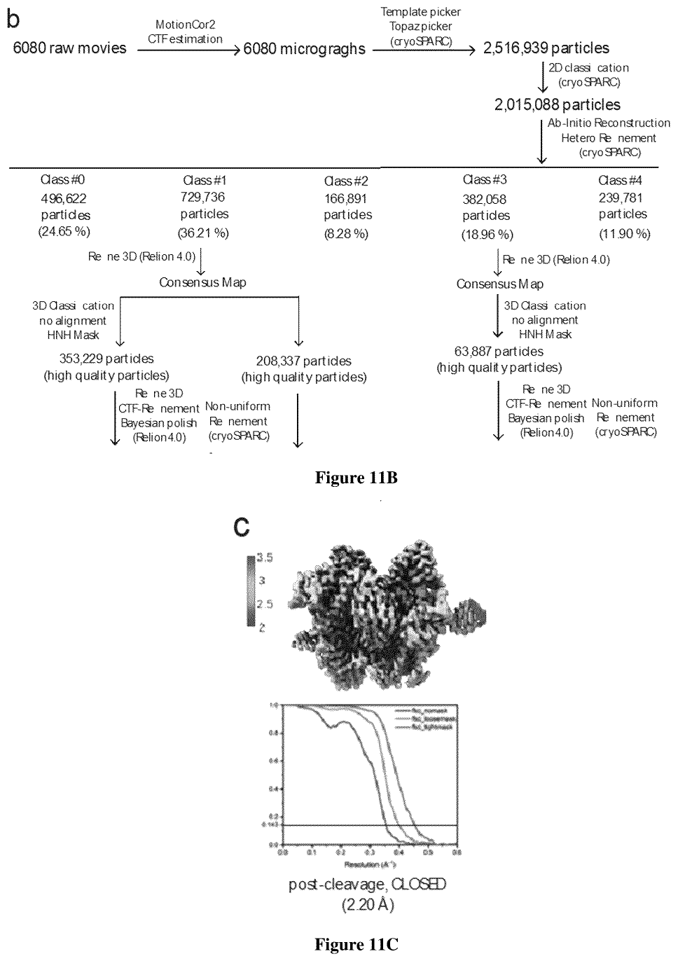

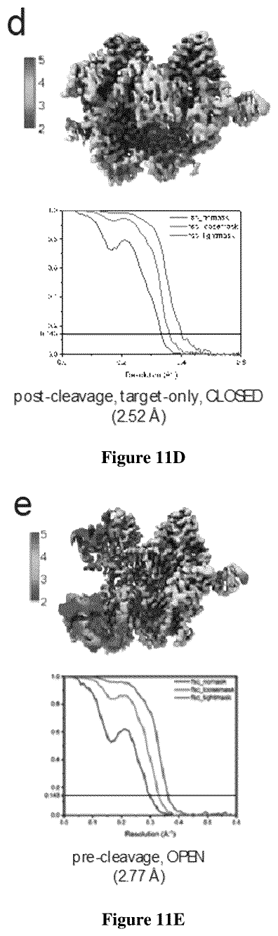

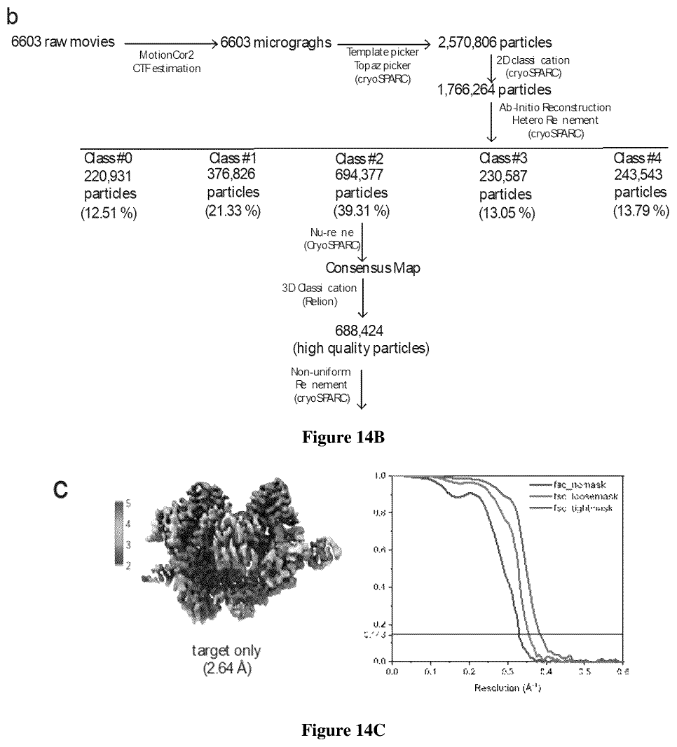

To understand the molecular basis of ThermoCas9 concerning its PAM and DNA methylation sensitivity as well as its catalytic efficiency, cryoEM structures of active ThermoCas9 bound with a DNA substrate associated with a 5′-NNGGCCA-3′ PAM (SEQ ID NO: 5) (Table 1, FIGS. 11, 12, and 13) were obtained. Active ThermoCas9 was assembled with the same DNA substrate associated with a 5′-NNGG^{5m}CCA-3′ PAM (SEQ ID NO: 5) (FIG. 14). The unmethylated DNA assembled complex resulted in three reconstructed structures at three functional states: the post-cleavage state at 2.2 Å resolution, the pre-cleavage state at 2.8 Å resolution, and a target DNA strand-only state at 2.5 Å, respectively (FIGS. 3A, 3B, 3C, 3D, 3E, 3F, 3G, Table 1 & FIG. 11). Consistent with a weaker interaction of ThermoCas9 for dsDNA with a methylated PAM, attempts to obtain such a complex resulted in complexes dominated by those bound with only the single target DNA strand despite the molar excess of the dsDNA used (FIG. 14).

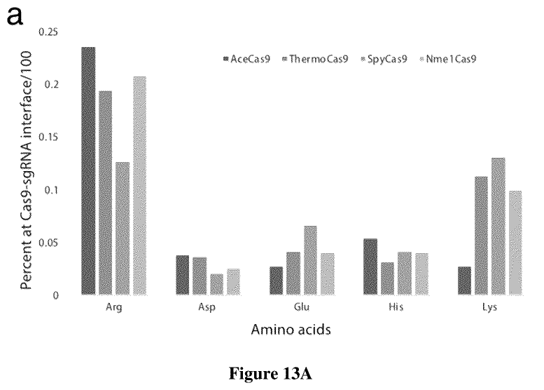

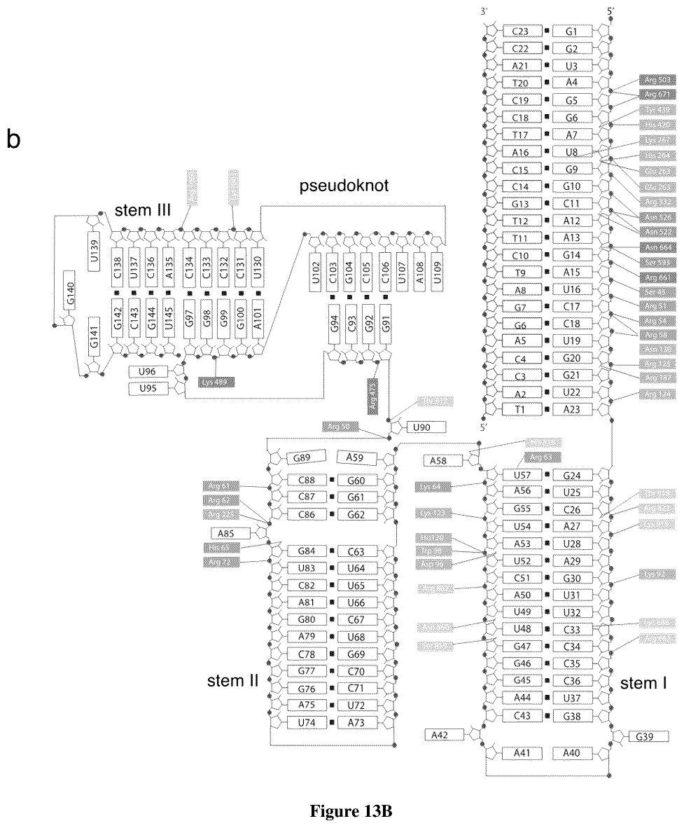

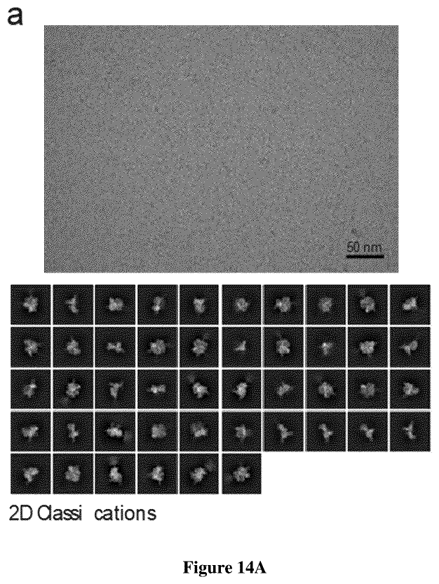

The overall architecture of ThermoCas9 resembles that of other Cas9 variants, with the typical nucleic acid recognition (REC) and the nuclease (NUC) lobes (FIG. 3A). Among those known, ThermoCas9 most closely resembles another Type II-C, the Cas9 of Neisseria meningitidis (NmelCas9; PDB 6JDV), with which it shares ˜39% amino acid sequence identity and identical size (1,082 aa). In contrast to NmelCas9, though, the guide RNA of ThermoCas9 folds differently in its 3′-terminal region. Residues 89-105 and 128-132 form a stable pseudoknot coaxially with stem loop III (133-145) (FIGS. 3B and 3C & FIG. 13B). The extended pseudoknot and stem loop III lie along the C-terminal extension of ThermoCas9 (FIG. 13B). Previous in vitro study showed that deletion of nucleotides 104-144, which would disrupt the pseudoknot, severely reduced DNA cleavage activity at high temperatures, showing a role in the thermostability of ThermoCas9. In addition, the extended Stem I interacts with the insertion elements to its PAM-interaction domain (PID) that are unique to ThermoCas9 (FIG. 13B). Interestingly, like other Type II-C Cas9 but unlike Type II-A Cas9, ThermoCas9 prefers arginine over lysine as RNA-binding residues (FIG. 13A).

Studies of other Cas9 nucleases have revealed the importance of domain motions, from an OPEN to a CLOSED conformation, in commencing its catalytic activities. After recognition of the correct PAM sequence by Cas9, the target DNA is slightly bent to position the target strand for base pairing with the guide RNA. Subsequent formation of the guide-target heteroduplex starting from the PAM-adjacent end (the seed) to the PAM-distal end gradually brings REC domains onto the guide-target heteroduplex until it is secured against the RuvC domain. This process coincides with a large swing of the HNH domain from the inactive (OPEN) to the active (CLOSED) configuration, which also adjusts the RuvC domain towards a cleavage-competent state. The captured pre- and post-cleavage conformations of ThermoCas9 are consistent with a similar activation process (FIGS. 3D, 3E, 3F, and 3G).

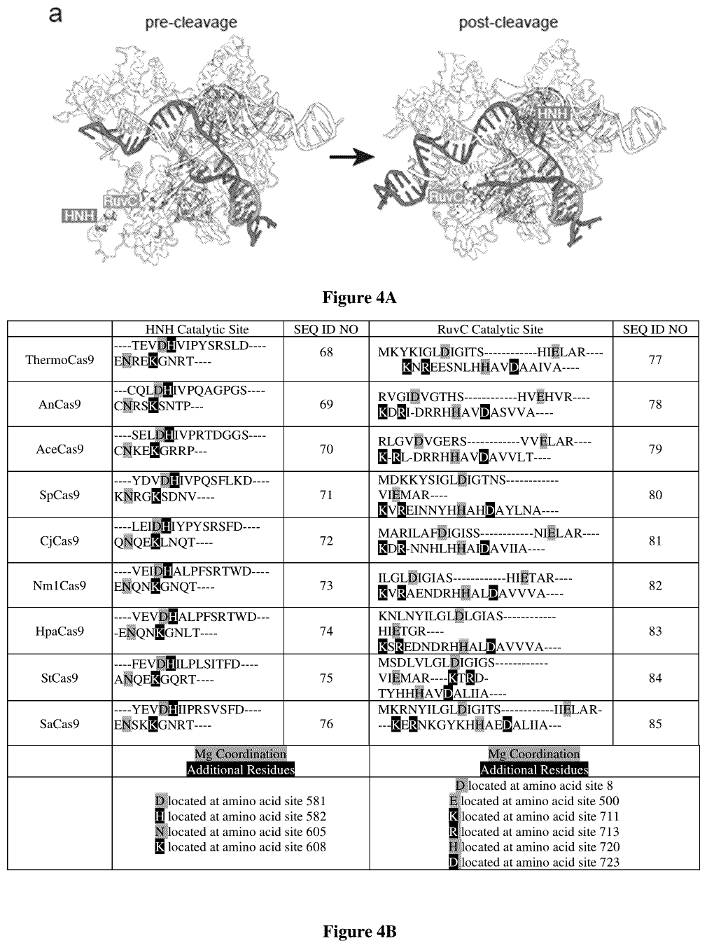

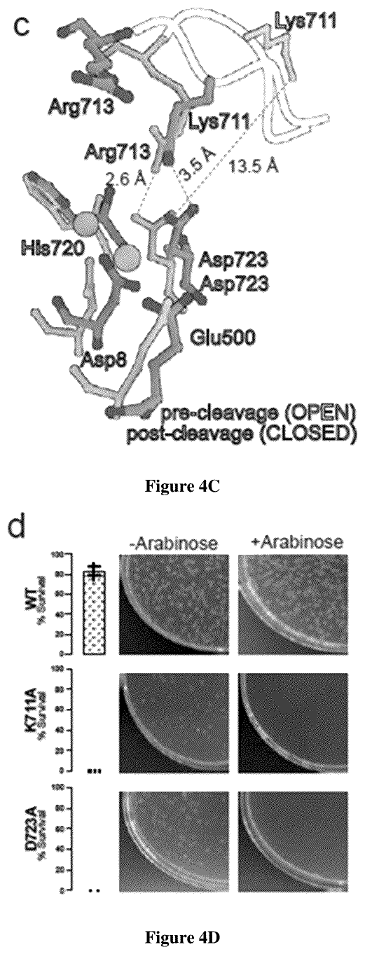

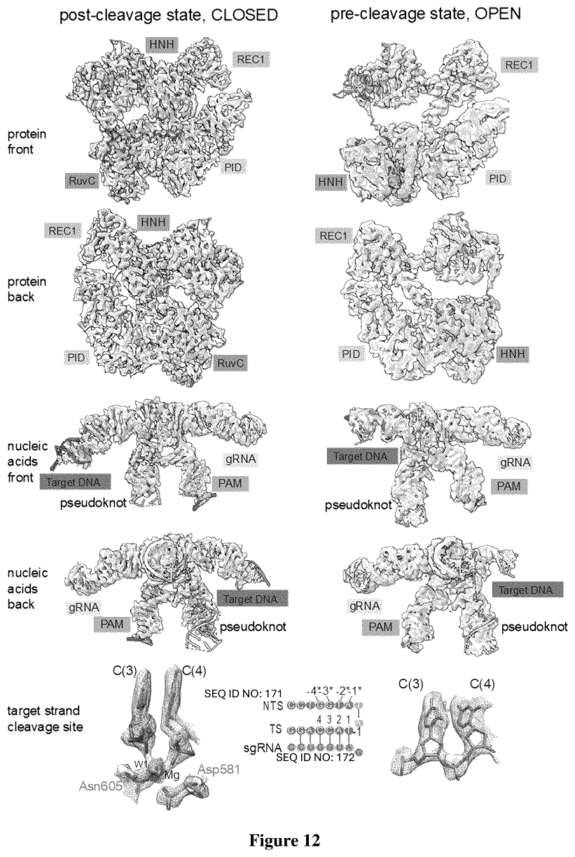

In the OPEN state, the target strand remains intact (FIG. 12), consistent with the fact that both the catalytic sites of both HNH domain and the RuvC domain are in an inactive state. The HNH nuclease domain is rested in a position ˜60 Å away from its cleavage site on the target DNA strand, while the REC2 domain loosely engages the guide: target heteroduplex (FIG. 3D, FIG. 3E, FIG. 4A). The region distal to PAM of the nontarget DNA strand is disordered and thus not placed into the RuvC active site (FIG. 3D, FIG. 3E, & FIG. 12).

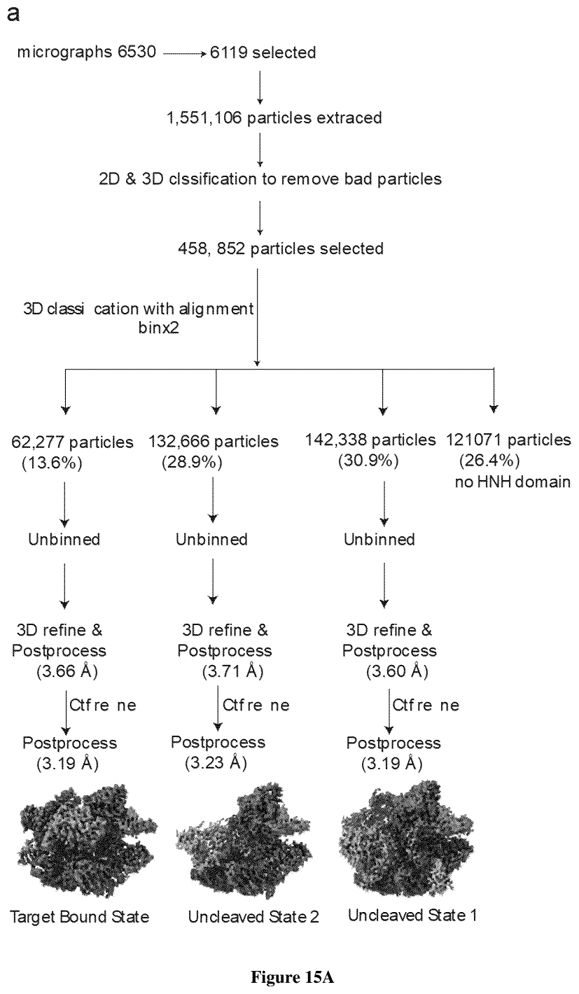

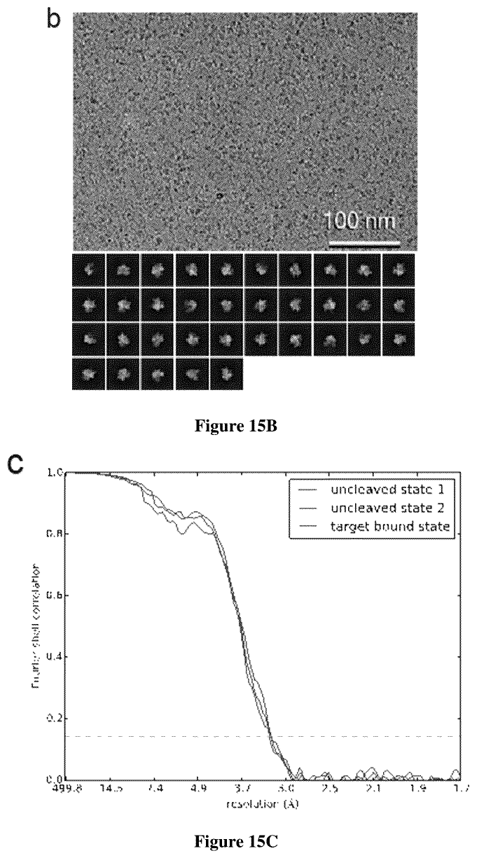

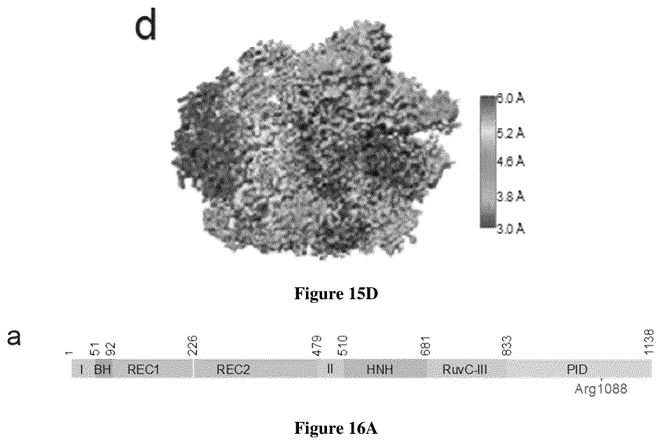

In the CLOSED state, the HNH domain has attacked the target strand, resulting in cleavage of the phosphodiester bond between nucleotides C3 and C4 (FIG. 3F, FIG. 3G, FIG. 4A, & FIG. 4B). Transition from the OPEN to the CLOSED conformation of ThermoCas9 requires a ˜ 180° rotation of the HNH domain. Similar to other Cas9 variants, this results in a cleavage-competent HNH site in which the conserved catalytic residues Asp581 and Asn605 coordinate with the catalytic magnesium ion (FIG. 4B). The leaving 3′-hydroxyl oxygen is cut from the scissile, but in the obtained structure it still remains coordinated with the magnesium at a distance of 2.2 Å (FIG. 4B). Together with the pro-Sp oxygen of the scissile phosphate and two water molecules, the six coordination ligands make a perfect octahedral geometry with the catalytic magnesium (FIG. 4B). In addition, Nd of His582 (equivalent to His840 of SpyCas9) maintains a close distance to the oxygen resulted from nucleophilic attack (2.35 Å), consist with its role in activating the nucleophilic water molecule. Interestingly, Lys608 (equivalent to Lys866 of SpyCas9), computationally predicted to activate His582, has a constant conformation throughout the OPEN to CLOSED transition process (FIG. 15), unlike Lys866 of SpyCas9 that undergoes a significant rearrangement, showing a different regulation process of HNH catalysis between the two enzymes.

Likewise, the RuvC active site lacks necessary metal ions and the substrate nontarget DNA in the OPEN state. In the CLOSED state, however, it forms a cleavage-competent configuration and has cleaved the phosphodiester bond between nucleotides G(−4*) and G(−3*) of the nontarget strand (FIG. 4B). The RuvC active center captured two magnesium ions that are coordinated with the pro-Sp oxygen of the scissile phosphate, the side chains of Asp8, Glu500, and His720 as well as four water molecules (FIG. 4B). The distance between the two magnesium ions is 3.8 Å, consistent with it being a state immediately following phosphodiester bond breakage. The observed structure of the RuvC active site confirms the importance of the universally conserved Glu500, Asp8, and His720 residues in coordinating the active site metals, similar to other Cas9 variants³⁸. However, unlike other Cas9 variants, ThermoCas9 undergoes a unique “OPEN-to-CLOSE” transition to activate its cleavage machinery (FIG. 4C). In the OPEN state, Asp723 forms an ion pair with Arg713 (2.6 Å) (equivalent to Asp986 and Arg976 of SpyCas9, respectively) while the nearby Lys711 (equivalent to Lys974 of SpyCas9) points away (FIG. 4C). In the CLOSED state, Asp723 breaks free from Arg713 and positions itself near the magnesium ions and their coordinated water molecules (FIG. 4B). Meanwhile, Lys711 makes ˜13 Å swing towards the cleavage site, interacting closely with the pro-Rp oxygen of the scissile phosphate (2.7 Å). This helps to stabilize the negatively charged state formed during cleavage. Though the equivalent residues of other Cas9 variants to Asp723, Lys711, and Arg713 of ThermoCas9 are well conserved (FIG. 4B) and similarly positioned in the CLOSED RuvC active site, they do not experience the similar transitions, showing a unique mechanism of regulation in ThermoCas9. Consistently, mutation of either Asp723 or Lys711 to alanine severely impaired ThermoCas9 activities in bacterial cells (FIG. 4D). These are the first demonstrated effects of the additional catalytic residues on Cas9 activity and highlight their importance in activation of the catalytic process.

### Structural Basis for DNA Methylation Sensitivity

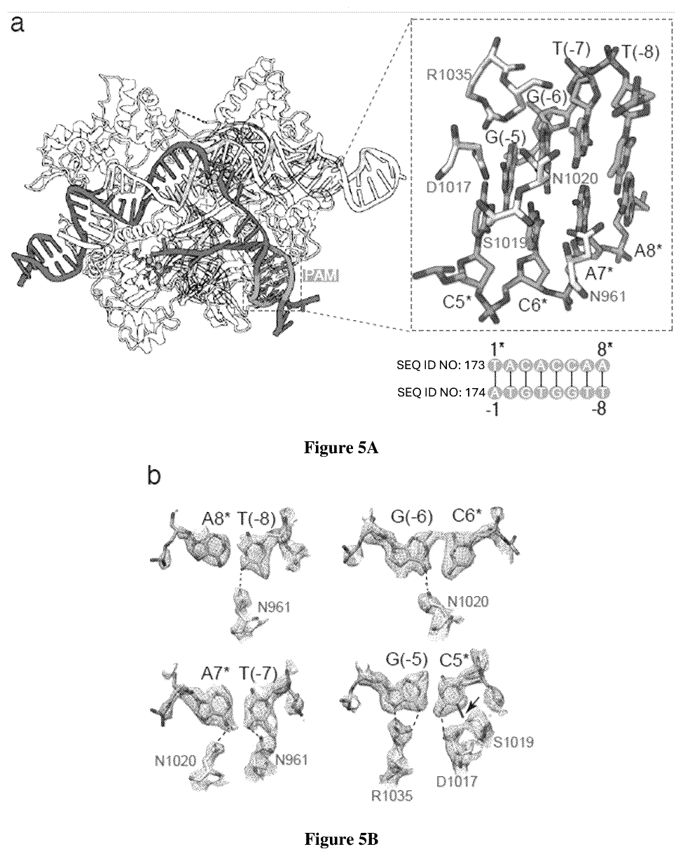

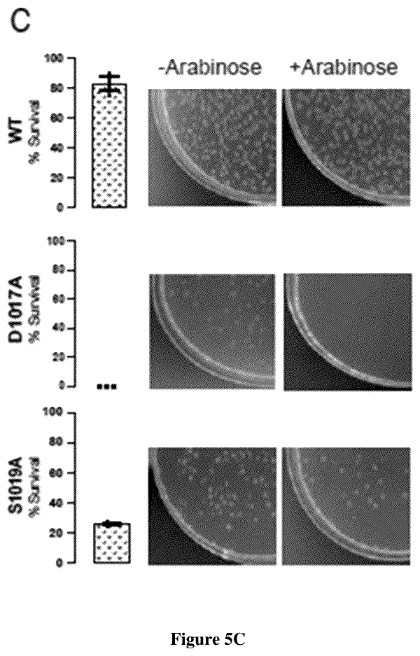

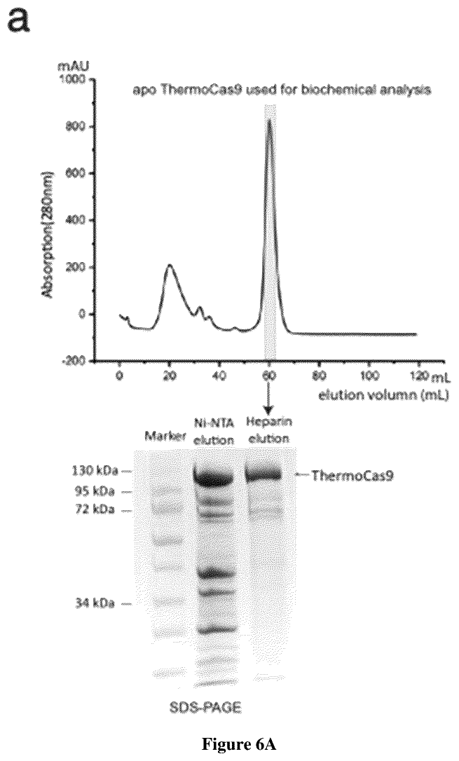

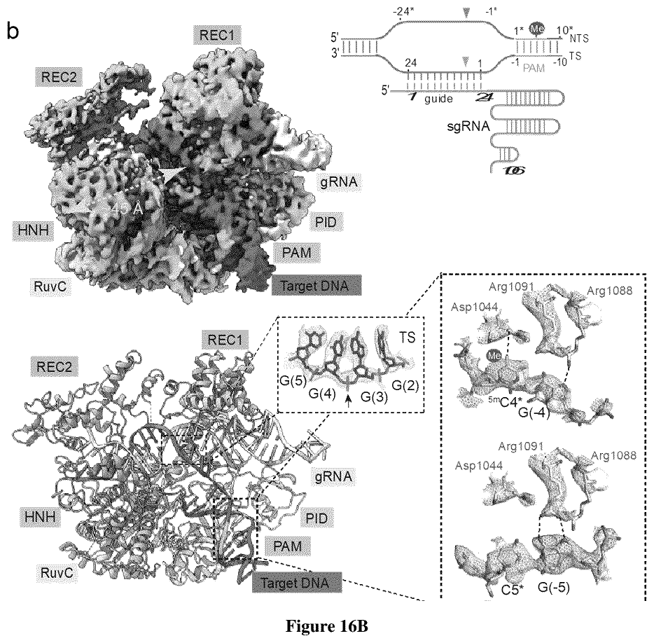

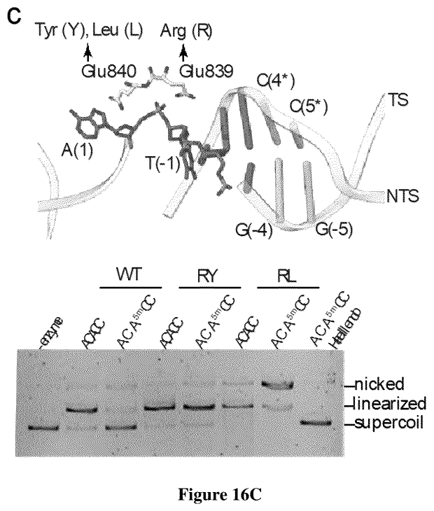

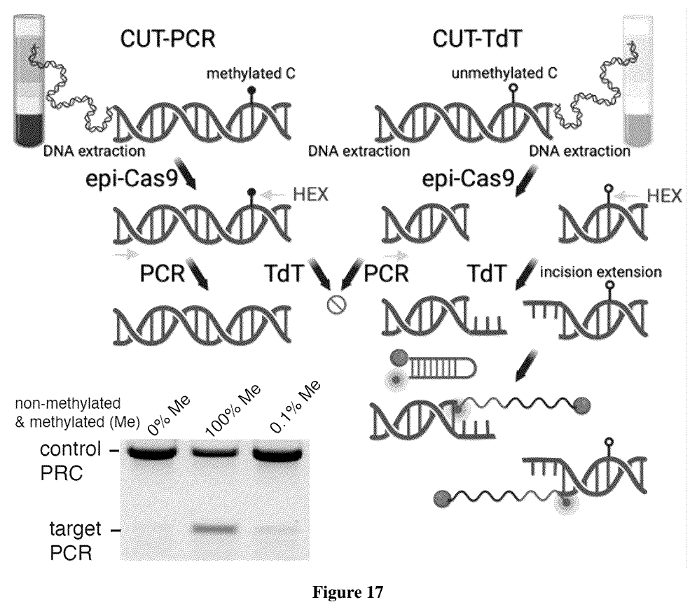

ThermoCas9 employs a PAM recognition method unique among the known Cas9s. It primarily focuses on recognition of the 5th base pair G(−5):C(5*) while imposing additional restrictions on the 6^{th}-8^{th} base pairs (FIG. 5A). Arg1035 employs a common mode of interaction observed in other Cas9s to form a pair of hydrogen bonds with G(−5). C(5*) is simultaneously recognized by Asp1017 and Ser1019 through its major groove edge, leaving little space for additional functional groups such as C5 methyl (FIG. 5B). The stringent interaction between ThermoCas9 and the G(−5):C(5*) pair explains why this is the most important base pair required for a PAM quality control check, and why C5 methylation is deleterious to PAM recognition. The close contact of Asn961 to T(−8) explains why this position prefers an AT pair while a lack of close contact supports little discrimination at position 7. The critical roles of Asp1017 and Ser1019 in C(5*) base recognition are revealed by activity assay results in bacterial cells (FIG. 5C). Mutation of Asp1017 to alanine virtually abolished DNA cleavage activity, while Ser1019 mutation retained partial activity. This demonstrates that Asp1017 is more critical to PAM recognition compared to Ser1019.

Whereas ThermoCas9 does not bind DNA containing a methylated PAM (FIG. 1C & FIG. 6), AceCas9 does form a stable complex with the PAM-methylated DNA. Thus, it was determined that cryoEM structures of AceCas9 were bound with a methylated DNA at 3.0 Å resolution (FIGS. 16 and 17 & Table 1). Strikingly, a large majority of particles formed from the active AceCas9 incubated with methylated DNA resulted in the pre-cleavage structure of AceCas9 where its HNH domain is rested far from the target strand cleavage site (FIG. 16). This result immediately shows that methylation in PAM inhibits HNH movement, a step critical to AceCas9 activation.

Comparison of structures of AceCas9 bound with dsDNA in the presence or the absence (PDB 8DKL) of PAM methylation does not show substantial structural changes, except for an increased mobility of the critical PAM-interacting Asp1044 and Arg1088 as indicated by weak density (FIG. 17B). This shows that methylation negatively impacts PAM recognition but it is insufficient to abolish DNA binding of AceCas9. It appears that the perturbation to PAM-interacting residues by methylation may prorogate to weaken the process of HNH placement. Mutation of the phosphate lock residues that was previously shown to overcome weaker 5′-NNNAC-3′ PAM 40 (SEQ ID NO: 9) alleviated inhibition by methylation (FIG. 17C). These findings demonstrate that AceCas9 activity is hindered by DNA methylation due to the disruption of PAM recognition. This provides a mechanistic explanation for the methylation-mediated regulation of AceCas9.

### DISCUSSION

It was found that ThermoCas9 has a stringent requirement for C: G pair at the 5th position of its PAM, which renders it inactive if the cytosine is methylated at carbon 5 (^{5m}C) both in vitro and in human cells. The structural characterization of ThermoCas9 was shown when bound with its DNA substrate and AceCas9 when bound with methylated DNA. The molecular basis of the inhibitory effect of PAM methylation differs in two nucleases: whereas ^{5m}C abolished the binding of ThermoCas9 to a dsDNA target, it prevented activation of AceCas9 when bound to a dsDNA. Along with AceCas9, these discoveries provide an opportunity to repurpose these Cas9 variants for epigenetic genome detection and manipulation.

The demonstrated methylation-dependent gene editing in human cells by ThermoCas9 expands the capability of the powerful CRISPR-Cas9 technology. Methylation-dependent editing can be used in targeted killing of lesion cells or in methylation-based diagnosis. This capability can enable RNA-guided detection of gain or loss of methylation sites in either genomic DNA or cell-free DNA (cfDNA) with high sensitivity at an early stage of disease onset.

Similar to other type II-C Cas9 variants, ThermoCas9 has weaker DNA cleavage activities than those of type II-A Cas9s such as SpyCas9. This is believed to be related to the weaker DNA unwinding activity by type II-C than type II-A Cas9 nucleases. While type II-C Cas9s may offer improved editing fidelity, which could be linked to their weaker catalytic efficiencies, efforts have been made in successfully improving catalytic efficiencies through enzyme engineering. It is thus possible to improve the catalytic efficiencies of ThermoCas9 for its broad applications in gene editing.

### Methods

### Cloning, Protein Expression and Purification

The DNA encoding ThermoCas9 with a C-terminal His6 tag was integrated into the pML-1B vector and expressed in Escherichia coli NiCo21 (DE3) strain. Cells were grown in Luria-Bertani (LB) medium with 0.2% D-(+)-glucose at 37° C. until optical density at 600 nm reached 0.8, at which point addition, isopropyl-β-D-thiogalactopyranoside (IPTG) was added to 0.5 mM concentration. Cells were grown for additional 16-18 hours at 20° C. and harvested by centrifugation and stored in −80° C. Previously frozen cells were lysed via sonication in a lysis buffer (500 mM NaCl, 50 mM Phosphate buffer pH 8.0 (sodium phosphate dibasic and sodium phosphate monobasic), 5 mM imidazole, 1 mM β-mercaptoethanol) containing 1 tablet of complete™ Mini Protease Inhibitor Cocktail (Sigma-Aldrich) per 100 mL. The lysate was centrifuged at a speed of 16,000 r.p.m. for 60 minutes at 4° C., after which the supernatant was loaded on a pre-equilibrated 5 mL HisTrap HP His tag protein purification column (Cytiva Life Sciences). The resin was subsequently washed with 200 mL wash buffer (500 mM NaCl, 50 mM Phosphate buffer pH8.0, 30 mM imidazole, 1 mM β-mercaptoethanol), before being eluted with elution buffer (500 mM NaCl, 50 mM Phosphate buffer pH 8.0, 250 mM imidazole, 1 mM β-mercaptoethanol). The resultant eluate was transferred onto a pre-equilibrated HiTrap Heparin HP affinity column (Cytiva Life Sciences) and eluted with a 100 mM-2 M NaCl gradient. The purified protein was then concentrated and stored at −80° C. until further use.

### In Vitro RNA Transcription

T7 in vitro transcription method was employed to produce the sgRNA for both ThermoCas9 and AceCas9. The sgRNA templates containing a T7 promotor were purchased from Eurofins Genomics (Louisville, KY). A 149 nt sgRNA for ThermoCas9 and a 106 nt sgRNA for AceCas9 (Table 2), respectively, were transcribed by T7 RNA polymerase in a transcription buffer (5 mM NTPs, 50 mM Tris-HCl pH 7.5, 15 mM MgCl₂, 5 mM dithiothreitol (DTT), 2 mM spermidine) and purified via the Monarch® RNA Cleanup Kits (New England Biolabs). The DNA employed in cryoEM and biochemical assays was purchased from Eurofins Genomics (Louisville, KY).

### Cryo-EM Sample Preparation, Data Collection, and 3D Reconstruction

The heparin purified protein was incubated with sgRNA at a 1:1.5 molar ratio at 37° C. for 30 min, and the resulting RNP was further purified via size exclusion chromatography with a Superdex 200 10/300 GL column (Cytiva Life Sciences) in gel filtration buffer (300 mM NaCl, 30 mM HEPES pH 7.5, 1 mM DTT). The Cas9-RNA-DNA ternary complex was assembled by adding pre-annealed substrate dsDNA into the RNP at a 2:1 molar ratio with the presence of 10 mM magnesium chloride. The reactive ternary complex was incubated at 37° C.-50° C. for 15-30 minutes. 4 μl of the sample was added to glow-discharged Gold 300 mesh R1.2/1.3 grids, which was then allowed to adsorb for 30 seconds prior to blotting for 2.5 seconds under conditions of 20° C. and 100% humidity. These grids were rapidly frozen in liquid nitrogen cooled ethane within Vitrobot Mark IV.

Raw micrographs were collected at the Laboratory for BioMolecular Structure (LBMS) of the Brookhaven National Laboratory using a Krios G3i cryo transmission electron microscope equipped with a Gatan K3 direct electron detector (ThermoFisher Scientific). Movies were recorded at a nominal magnification of 105,000 in a super-resolution mode with an energy filter of 15 eV, corresponding to a corrected physical pixel size of 0.82 Å/pixel. A total dose of 60 e⁻/Å² was spread over 60 frames with random defocus set to −0.8 to −2.5 μm. Motion correction was executed in bin 2 via MotionCorr2 and contrast transfer function (CTF) estimation was carried out with Getf. A total of 6080 micrographs were collected and 2,516,939 particles were picked using Topaz, followed by multiple rounds of 2D classification using CryoSPARC, resulting in 2,015,088 good particles for 3D classification. After heterogenous refinement in CryoSPARC, the data set was classified into 5 classes. Several rounds of 3D refinement and 3D classification were then performed using Relion 4.0 to obtain high-quality particles. Finally, several rounds of non-uniform refinement were performed using CryoSPARC to reach the final 3D structures. Structural models were built in COOT and refined in PHENIX to satisfactory stereochemistry and real space map correlation parameters. Note that water molecules were only modeled based on both density and interaction chemistry in the two high-resolution structures.

### Bacterial Survival Assay

The survival assay in bacterial cells followed a previously outlined procedure with minor modifications. In brief, electrocompetent E. coli BW25141, harboring the modified p11-LacY-wtx1 plasmid encoding toxic ccdB protein, were transformed with 60 ng of wild-type or mutant ThermoCas9 plasmids. Afterward, the cells were recovered in LB for 30 minutes with shaking at 37° C. Subsequently, 0.05 mM IPTG was introduced, and the recovery process continued for an additional 60 minutes. The recovered cells were then plated on LB agar plates containing either chloramphenicol (15 mg/mL) or a combination of chloramphenicol and 10 mM arabinose. The plates were incubated at 37° C. for 16-20 hours. Manual counting of colonies was performed on both plates, and survival rates were determined by dividing the colony forming units on arabinose-containing plates by those on chloramphenicol-only plates.

### In Vitro DNA Cleavage Assay and Competition Assay

ThermoCas9 was combined with sgRNA at a 1:1 ratio and incubated at 37° C. for 30 minutes to form the RNP. The target plasmid at 6 nM was then added to the RNP at 1 μM and allowed to incubate for varying lengths of time. The reactions were stopped by adding a 5× stop buffer (25 mM Tris pH 7.5, 250 mM EDTA pH 8.0, 1% SDS, 0.05% w/v bromophenol blue, and 30% glycerol). The reaction products were separated on a 0.8% agarose gel and stained by ethidium bromide.

Fluorescently labelled oligonucleotides were also used to prepare DNA substrates. 6-carboxyfluorescein (FAM)-labelled NTS DNA was annealed with an unlabeled TS DNA at a 1:1 molar ratio. Separately, hexachlorofluorescein (HEX)-labelled TS DNA was annealed with unlabeled NTS DNA at a 1:19 molar ratio. Annealing was performed by heating the DNA mixtures to 75° C. for 5 minutes, followed by a gradual cooling to room temperature. Pre-annealed double-stranded DNA (dsDNA) substrates were prepared at concentrations of 100 nM-200 nM for the labelled strand. These substrates were then added to a ThermoCas9 RNP solution at 1 μM to initiate the cutting reaction. Divalent metal ions, specifically 10 mM of MgCl₂, were also included in each reaction. The reaction mixtures were incubated at 37° C.-50° C. for a duration of 1 hour before being quenched by the addition of 2× RNA loading buffer (97% formamide, 0.02% SDS, and 1 mM EDTA). The reaction products were resolved using a 7 M urea 20% polyacrylamide denaturing gel. Gel electrophoresis was performed under denaturing conditions to ensure the separation of DNA fragments based on size. Following electrophoresis, the gel was visualized using a Bio-Rad ChemiDoc gel imaging system. Fluorescent labels were detected using excitation wavelengths of 488 nm for FAM and 580 nm for HEX, respectively.

For competition assays, ThermoCas9 RNP at 1 μM was mixed with the target plasmid at 10 nM, and a competing oligo DNA substrate at concentrations of 50 nM-1 μM. The reactions were incubated at 50° C. for 15 minutes and stopped by adding the 5× stop buffer. The reaction products were separated on a 0.8% agarose gel and stained by ethidium bromide.

### In Silico Analysis of Differentially Methylated Sites in HEK293 and HCT116 Cells

Reduced representation bisulfite sequencing (RRBS) data was collected from the ENCODE functional genomics database for various cells. The call sets were downloaded from the ENCODE portal with the following identifiers: ENCFF001TMQ, ENCFF001TMM, ENCFF001TLC, ENCFF001TOH, ENCFF001TOW, ENCFF001TMU, ENCFF001TOZ, ENCFF001TQE, ENCFF001TQA, and ENCFF001TLG. An in-house program was used to compare the methylation profiles based on the methylation scores. The RRBS data was utilized to map genes with different methylation profiles in the Integrative Genomics Viewer.

### Transfections and Gene Editing in HEK293 and HCT116 Cells

Human-codon optimized thermocas9-sv40nls gene and its sgRNA module were expressed under the control of the constitutive cytomegalovirus (P_{CMV}) and U6 RNA polymerase III (P_{U6}) promoters, respectively. The EGFP reporter gene was coexpressed under the constitutive elongation factor-1 alpha promoter (P_{EF-1α}) to allow for sorting of the successfully transfected cells, as previously described. Four spacers were designed that target protospacers in the chromosomal genes VEGFA, EMX1, and PRDX4. All differentially methylated protospacers were flanked by a PAM of (5′-NNNNCGAA-3′) (SEQ ID NO: 7) thus representing a CpG methylated PAM. The targeting spacers of EMX1 and PRDX4 are differentially methylated in the PAM sequence between HEK293 and HCT116; the negative and positive control targets are located on the VEGFA gene. HCT116 cells were maintained in McCoy's 5A media and HEK293 cells were maintained in DMEM media supplemented with 10% fetal bovine serum and 1% penicillin/streptomycin at 37° C. with 5% CO₂. Both HEK293 and HCT116 cells were seeded on physically surface-treated 24-well plates (Corning/Falcon) at a seeding density of 1.0×10⁵ cells per well. After 24 hours of incubation, 0.5 μg of genome editing plasmid was transfected into the HEK293 and HCT116 cells using Lipofectamine™ 3000 Transfection reagent (Thermo Fisher, L3000015). For each well on the plate, transfection plasmids were combined with OptiMEM Reduced Serum Medium (Thermo Fisher, 31985062) to a total volume of 25 μl and mixed with 1 μl P3000 reagent. Separately, 25 μl OptiMEM was combined with 1.1 μl Lipofectamine 3000 reagent. Plasmid and Lipofectamine solutions were then combined, incubated at room temperature for 10 min and pipetted on to cells. The transfected cells were cultured 72 hours and further evaluated for the presence of GFP using Fluorescence Activated Cell Sorting (FACS).

### Flow Cytometry

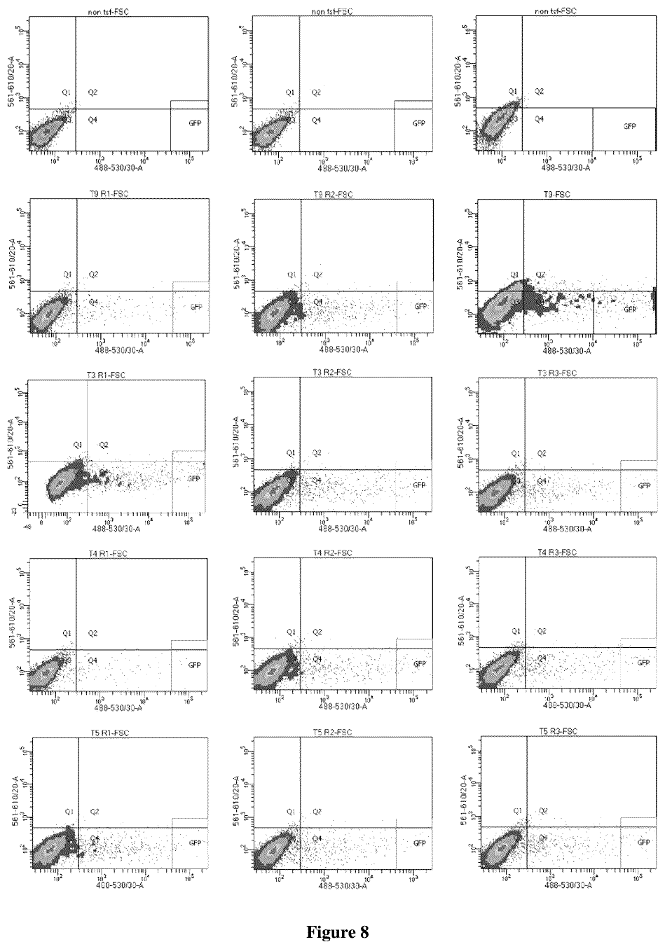

After 72 hours of incubation, the transfected HEK293 and HCT116 cells were harvested, centrifuged at 1000 r.p.m. for 5 minutes, resuspended in 250 μL DMEM (10% FBS and 1% pen/strep), and filtered through Nylon Mesh 52 micron, 32% open area filter (Component Supply Co.). GFP+ fluorescent cells were bulk sorted using the BD FACSAria III cell sorter device (BD) (488 nm laser, FITC detection channel for GFP fluorescence). The cells were gated for ‘high-green’ to reduce the signal to noise of auto-fluorescent cells (FIGS. 7-8). Cells were transferred to a 96-well nucleon plate and centrifuged at 200 r.p.m. for 2 minutes and cultured for ˜1-2 weeks (37° C.; 5% CO₂). When ˜75% confluency was reached, the propagated cells of each experiment were steadily passaged to 24-well plates and further screened for indels.

### Screening for Genome Editing

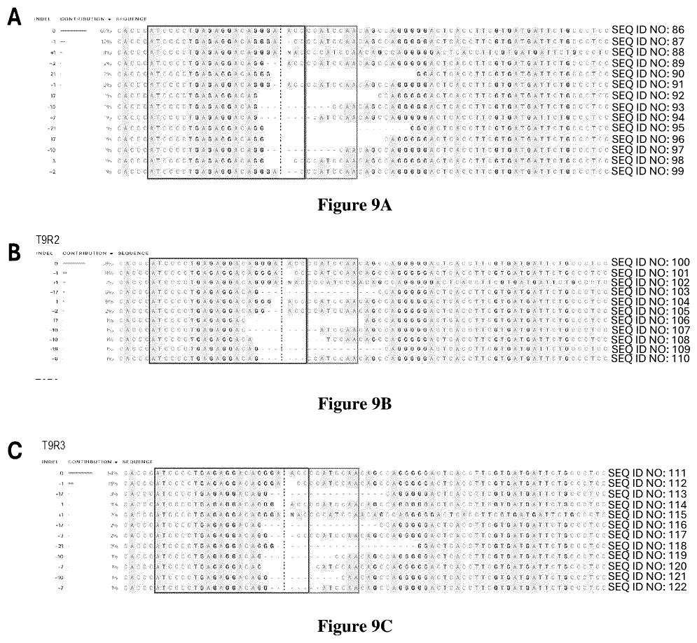

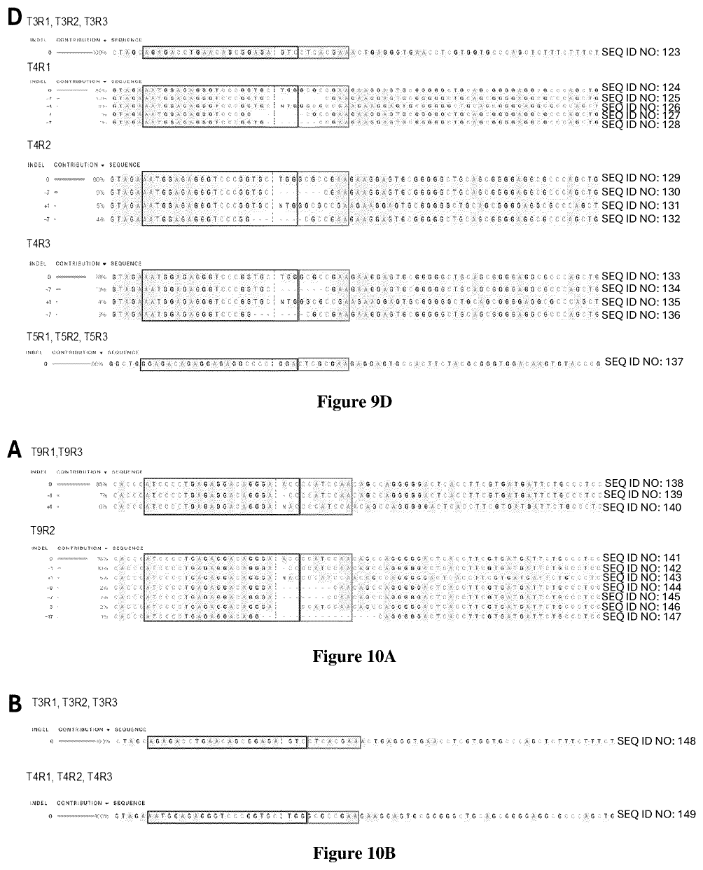

HEK293 and HCT116 genomic DNA was isolated from the bulk population of propagated cells grown ˜2-3 weeks post-FACS sorting. Genomic DNA was extracted using the Zymo Research Quick-DNA MicroPrep kit. Genomic target regions (VEGFA, EMX1, and PRDX4) were PCR amplified with Q5® High-Fidelity 2× Master Mix (New England Biolabs). The PCR products were verified on a 2% DNA agarose gel, and they were subsequently gel purified with the E.Z.N.A. gel extraction kit (Omega-BioTek). To detect indel formation, the gel purified PCR products were subjected to Sanger sequencing (FSU sequencing facility). The sequencing results of the genome editing assays were analyzed using the Inference of CRISPR Edits tool (ICE, Synthego) (FIGS. 9 & 10).

### Bisulfite Sequencing

Genomic DNA of both HEK293 and HCT116 were bisulfite treated via the EpiJET Bisulfite Conversion Kit (Thermo Scientific, K1461) following manufacturer's instructions. The MethPrimer online tool was utilized to design primers to amplify bisulfite-converted samples flanking the regions of gene editing targets followed by Sanger sequencing (FSU sequencing facility).

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLES
Table 1. Cryo-EM data collection, refinement and validation statistics

| | ThermoCas9- | ThermoCas9- | ThermoCas9- | ThermoCas9- | AceCas9- |
|---|---|---|---|---|---|
| | CpC DNA | CpC DNA | CpC DNA | ^{5m}CpC DNA | ^{5m}CpC DNA |
| | Post-cleavage | Pre-cleavage | target-only | target-only | Pre-cleavage |
| | (EMDB-xxxx) | (EMDB-xxxx) | (EMDB-xxxx) | (EMDB-N.A.) | (EMDB-xxxx) |
| | (PDB xxxx) | (PDB xxxx) | PDB xxxx) | (PDB N.A.) | (PDB xxxx) |
| Data collection and | | | | | |
| processing | | | | | |
| Magnification | 105K | 105K | 105K | 105K | 105K |
| Voltage (kV) | 300 kV | 300 kV | 300 kV | 300 kV | 300 kV |
| Electron exposure | 60 | 60 | 60 | 60 | 60 |
| (e−/Å²) | | | | | |
| Defocus range (μm) | (−0.8)-(−2.5) | (−0.8)-(−2.5) | (−0.8)-(−2.5) | (−1.0)-(−2.5) | (−1.0)-(−2.5) |
| Pixel size (Å) | 0.82 | 0.82 | 0.82 | 0.82 | 0.82 |
| Symmetry imposed | C1 | C1 | C1 | C1 | C1 |
| Initial particle images | 6080 | 6080 | 6080 | 6603 | 6530 |
| (no.) | | | | | |
| Final particle images | 6080 | 6080 | 6080 | 6603 | 6119 |
| (no.) | | | | | |
| Map resolution (Å) | 2.20 | 2.77 | 2.52 | 2.64 | 2.88 |
| FSC threshold | 0.143 | 0.143 | 0.143 | 0.143 | 0.143 |
| Refinement | | | | | |
| Initial model used | N.A. | N.A. | N.A | Not | 8D2N |
| (PDB code) | | | | refined | |
| Model resolution (Å) | | | | | |
| FSC threshold | 0.0/0.143/0.5 | 0.0/0.143/0.5 | 0.0/0.143/0.5 | | 0.0/0.143/0.5 |
| Model resolution | 2.2-2.6 | 2.7-3.0 | 2.5-2.9 | | 2.8-3.1 |
| range (Å) | | | | | |
| Map sharpening B | 66.5 | 72.6 | 89.9 | | 126.1 |
| factor (Å²) | | | | | |
| Map correlation | 0.77 | 0.80 | 0.79 | | 0.76 |
| coefficient | | | | | |
| Model composition | | | | | |
| Non-hydrogen atoms | 122442 | 12166 | 11603 | | 111415 |
| Protein residues | 1019 | 1064 | 1019 | | 1105 |
| Nucleotide residues | 180 | 161 | 149 | | 128 |
| Ligands | 7 | 2 | 5 | | 0 |
| Water | 21 | 2 | 19 | | 0 |
| B factors (Å²) | | | | | |
| Protein | 0.0/119.6/48.2 | 23.5/219.3/115.7 | 0.0/97.8/41.2 | | 5.0/135.0/73.4 |
| (min/max/mean) | | | | | |
| Nucleotides | 0.0/136.6/47.5 | 6.3/175.8/80.1 | 0.0/155.8/29.0 | | 13.5/182.1/99.2 |
| (min/max/mean) | | | | | |
| Ligand | 23.7/58.4/42.6 | 55.0/65.9/60.5 | 0.16/44.5/27.7 | | — |
| (min/max/mean) | | | | | |
| Water | 13.2/46.3/31.8 | 44.0/46.3/45.2 | 0.1/57.8/20.0 | | — |
| (min/max/mean) | | | | | |
| R.m.s. deviations | | | | | |
| Bond lengths (Å) | 0.005 | 0.005 | 0.005 | | 0.003 |
| Bond angles (°) | 1.031 | 0.764 | 0.849 | | 0.635 |
| Validation | | | | | |
| MolProbity score | 1.55 | 1.75 | 1.41 | | 1.69 |
| Clashscore | 4.97 | 5.79 | 4.60 | | 8.33 |
| Poor rotamers (%) | 0.00 | 0.00 | 0.00 | | 0.00 |
| Ramachandran plot | | | | | |
| Favored (%) | 95.9 | 93.4 | 96.9 | | 96.4 |
| Allowed (%) | 4.1 | 6.6 | 3.1 | | 3.6 |
| Disallowed (%) | 0.0 | 0.0 | 0.0 | | 0.0 |

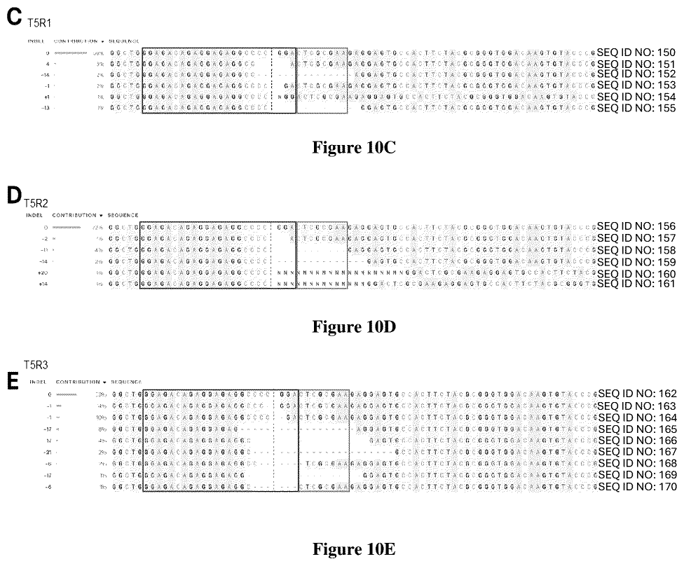

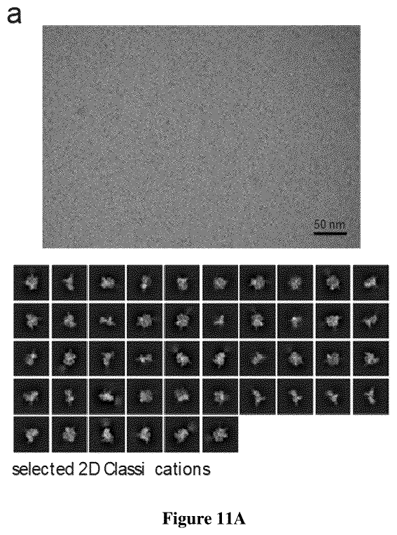

TABLE 2
Oligos Used in Studies

| Description | Sequence (5′-3′) | Used For |
|---|---|---|
| Forward primer | AGGGGAAATAAGGGATATTTTTTTTA | Bisulfite-converted |
| VEGFA bisulfite | (SEQ ID NO: 10) | gDNA |
| sequencing in | | amplification prior |
| HEK293 cell line | | to sequencing |
| Reverse primer | CCAAAATCTCCTCCTAAATTTTTAC | Bisulfite-converted |
| VEGFA bisulfite | (SEQ ID NO: 11) | gDNA |
| sequencing in | | amplification prior |
| HEK293 cell line | | to sequencing |
| Forward primer | GGGGAAATAAGGGATATTTTTTTTA | Bisulfite-converted |
| VEGFA bisulfite | (SEQ ID NO: 12) | gDNA |
| sequencing in | | amplification prior |
| HCT116 cell line | | to sequencing |
| Reverse primer | AACCCCCAACATCTAATTAATCTTTA | Bisulfite-converted |
| VEGFA bisulfite | (SEQ ID NO: 13) | gDNA |
| sequencing in | | amplification prior |
| HCT116 cell line | | to sequencing |
| Forward primer | TTGTTTTTTTGGGAGGGAGAT | Bisulfite-converted |
| EMX1 bisulfite | (SEQ ID NO: 14) | gDNA |
| sequencing | | amplification prior |
| | | to sequencing |
| Reverse primer | CATAAAATCCTTAATAACCAAAAAC | Bisulfite-converted |
| EMX1 bisulfite | (SEQ ID NO: 15) | gDNA |
| sequencing | | amplification prior |
| | | to sequencing |
| Forward primer | GGTTGGAGTTGTGTAGGGTTG | Bisulfite-converted |
| PRDX4 bisulfite | (SEQ ID NO: 16) | gDNA |
| sequencing | | amplification prior |
| | | to sequencing |
| Reverse primer | CCTCTTCTAAAATCTAATCAATAAAAACC | Bisulfite-converted |
| PRDX4 bisulfite | (SEQ ID NO: 17) | gDNA |
| sequencing | | amplification prior |
| | | to sequencing |
| Forward primer | GGAAGGGAGGAGAGATCCCA | Amplification of |
| VEGFA | (SEQ ID NO: 18) | gene-edited gDNA |
| | | prior to sequencing |
| | | & ICE analysis |
| Reverse primer | TACCATGGCTTTGGACCAGG | Amplification of |
| VEGFA | (SEQ ID NO: 19) | gene-edited gDNA |
| | | prior to sequencing |
| Forward primer | AGCGGGAGGAGTTGTACTCT | Amplification of |
| EMX1 | (SEQ ID NO: 20) | gene-edited gDNA |
| | | prior to sequencing |
| | | & ICE analysis |
| Reverse primer | GGGGGAGGTGAGGGGTG | Amplification of |
| EMX1 | (SEQ ID NO: 21) | gene-edited gDNA |
| | | prior to sequencing |
| Forward primer | ATGCAAGCCATTCACAAGCA | Amplification of |
| PRDX4 | (SEQ ID NO: 22) | gene-edited gDNA |
| | | prior to sequencing |
| | | & ICE analysis |
| Reverse primer | ATGCAAACTCGGGACTAGGG | Amplification of |
| PRDX4 | (SEQ ID NO: 23) | gene-edited gDNA |
| | | prior to sequencing |
| ThermoCas9 sgRNA | AAAACGCCTAAGAGTGGGGAATGCCCGA | In-vitro |
| in-vitro transcription | AGAAAGCGGGCGATAGGCGATCCCCAAC | transcription of |
| template strand | GCCACGGGTCAGTCTGCCTATAGGCAGA | ThermoCas9 |
| | AAGCCCTTATCATAGTAACCCTGTTTCCA | sgRNA |
| | GGGGAACTATGACTACCAGGATCTTGCC | |
| | ATCCTACCTATAGTGAGTCGTATTA | |
| | (SEQ ID NO: 24) | |
| T7 promoter oligo | TAATACGACTCACTATA | In-vitro |
| | (SEQ ID NO: 25) | transcription of |
| | | ThermoCas9 |
| | | sgRNA |
| PAM CC target | AGCTTCCTGTATACCAGGATCTTGCCATC | Substrate of |
| strand | CTACCTCTAGA (SEQ ID NO: 26) | ThermoCas9 in- |
| | | vitro cleavage |
| | | assay |
| PAM CC non-target | TCTAGAGGTAGGATGGCAAGATCCTGGT | Substrate of |
| strand | ATACACCAAGCT (SEQ ID NO: 27) | ThermoCas9 in- |
| | | vitro cleavage |
| | | assay |
| PAM CG target | AGCTTCGTGTATACCAGGATCTTGCCATC | Substrate of |
| strand | CTACCTCTAGA (SEQ ID NO: 28) | ThermoCas9 in- |
| | | vitro cleavage |
| | | assay |
| PAM CG non-target | TCTAGAGGTAGGATGGCAAGATCCTGGT | Substrate of |
| strand | ATACACGAAGCT (SEQ ID NO: 29) | ThermoCas9 in- |
| | | vitro cleavage |
| | | assay |
| Methylated PAM | AGCTT[5Me~dC]GTGTATACCAGGATCTTG | Substrate of |
| CG target strand | CCATCCTACCTCTAGA | ThermoCas9 in- |
| | (SEQ ID NO: 30) | vitro cleavage |
| | | assay |
| Methylated PAM | TCTAGAGGTAGGATGGCAAGATCCTGGT | Substrate of |
| CG non-target strand | ATACA[5Me~dC]GAAGCT | ThermoCas9 in- |
| | (SEQ ID NO: 31) | vitro cleavage |
| | | assay |
| HEX-labelled PAM | [5~HEX]AGCTTCGTGTATACCAGGATCTT | Fluorescence- |
| CG target strand | GCCATCCTACCTCTAGA | labelled substrate |
| | (SEQ ID NO: 32) | of ThermoCas9 in- |
| | | vitro cleavage |
| | | assay |
| HEX-labelled | [5~HEX]AGCTT[5Me~dC]GTGTATACCAGG | Fluorescence- |
| methylated PAM CG | ATCTTGCCATCCTACCTCTAGA | labelled substrate |
| target strand | (SEQ ID NO: 33) | of ThermoCas9 in- |
| | | vitro cleavage |
| | | assay |
| FAM-labelled PAM | [6~FAM]TCTAGAGGTAGGATGGCAAGATC | Fluorescence- |
| CC non-target strand | CTGGTATACACCAAGCT | labelled substrate |
| | (SEQ ID NO: 34) | of ThermoCas9 in- |
| | | vitro cleavage |
| | | assay |
| FAM-labelled PAM | [6~FAM]TCTAGAGGTAGGATGGCAAGATC | Fluorescence- |
| CG non-target strand | CTGGTATACACGAAGCT | labelled substrate |
| | (SEQ ID NO: 35) | of ThermoCas9 in- |
| | | vitro cleavage |
| | | assay |
| FAM-labelled | [6~FAM]TCTAGAGGTAGGATGGCAAGATC | Fluorescence- |
| methylated PAM CG | CTGGTATACA[5Me~dC]GAAGCT | labelled substrate |
| non-target strand | (SEQ ID NO: 36) | of ThermoCas9 in- |
| | | vitro cleavage |
| | | assay |
| K711A-F | GAATTTTAACGCAAACCGGGAAGAATCG | Q5 site directed |
| | AATTTG (SEQ ID NO: 37) | mutagenesis of |
| | | ThermoCas9 |
| K711A-R | CAACGGCTGCGTAAATGG | Q5 site directed |
| | (SEQ ID NO: 38) | mutagenesis of |
| | | ThermoCas9 |
| D723A-F | CATGCCGTCGCTGCTGCCATC | Q5 site directed |
| | (SEQ ID NO: 53) | mutagenesis of |
| | | ThermoCas9 |
| D723A-R | ATGCAAATTCGATTCTTCCCGG | Q5 site directed |
| | (SEQ ID NO: 39) | mutagenesis of |
| | | ThermoCas9 |
| D1017A-F | CAAACCATCGCCTCCTCCAATG | Q5 site directed |
| | (SEQ ID NO: 40) | mutagenesis of |
| | | ThermoCas9 |
| D1017A-R | ATAATAGGCGAACAGATC | Q5 site directed |
| | (SEQ ID NO: 41) | mutagenesis of |
| | | ThermoCas9 |
| S1019A-F | CATCGACTCCGCCAATGGAGG | Q5 site directed |
| | (SEQ ID NO: 42) | mutagenesis of |
| | | ThermoCas9 |
| S1019A-R | GTTTGATAATAGGCGAACAGATC | Q5 site directed |
| | (SEQ ID NO: 43) | mutagenesis of |
| | | ThermoCas9 |
| T9 gRNA target | ACCGATCCCCTGAGAGGACAGGGAACC | Golden gate |
| | (SEQ ID NO: 44) | assembly cloning |
| | | using SapI |
| T9 gRNA reverse | GACGGTTCCCTGTCCTCTCAGGGGATC | Golden gate |
| complement | (SEQ ID NO: 45) | assembly cloning |
| | | using SapI |
| T3 gRNA target | ACCGAGAGACCTGAACAGCGGAGAGTC | Golden gate |
| | (SEQ ID NO: 46) | assembly cloning |
| | | using SapI |
| T3 gRNA reverse | GACGACTCTCCGCTGTTCAGGTCTCTC | Golden gate |
| complement | (SEQ ID NO: 47) | assembly cloning |
| | | using SapI |
| T4 gRNA target | ACCGAATGGAGAGGGTCCCGGTGCTGG | Golden gate |
| | (SEQ ID NO: 48) | assembly cloning |
| | | using SapI |
| T4 gRNA reverse | GACCCAGCACCGGGACCCTCTCCATTC | Golden gate |
| complement | (SEQ ID NO: 49) | assembly cloning |
| | | using SapI |
| T5 gRNA target | ACCGGAGACAGAGGAGAGGCCCCGGA | Golden gate |
| | (SEQ ID NO: 50) | assembly cloning |
| | | using SapI |
| T5 gRNA reverse | GACTCCGGGGCCTCTCCTCTGTCTCC | Golden gate |
| complement | (SEQ ID NO: 51) | assembly cloning |
| | | using SapI |

| | SEQUENCES |
|---|---|
| 1. | SEQ ID NO: 1 - PAM |
| | NNNNCG |
| | (N represents any nucleotide selected from adenine (A), thymine (T), |
| | guanine (G), or cytosine (C)) |
| 2. | SEQ ID NO: 2 - PAM |
| | NNNNCC |
| | (N represents any nucleotide selected from adenine (A), thymine (T), |
| | guanine (G), or cytosine (C)) |
| 3. | SEQ ID NO: 3 - PAM |
| | NNNNCNR |
| | (N represents any nucleotide selected from adenine (A), thymine (T), |
| | guanine (G), or cytosine (C); R represents a purine nucleotide) |
| 4. | SEQ ID NO: 4 - optimal PAM |
| | NNNNCCAA |
| | (N represents any nucleotide selected from adenine (A), thymine (T), |
| | guanine (G), or cytosine (C)) |
| 5. | SEQ ID NO: 5 - CpC-containing PAM |
| | NNGGCCA |
| | (N represents any nucleotide selected from adenine (A), thymine (T), |
| | guanine (G), or cytosine (C)) |
| 6. | SEQ ID NO: 6 - CpG-containing PAM |
| | NNNNCGA |
| | (N represents any nucleotide selected from adenine (A), thymine (T), |
| | guanine (G), or cytosine (C)) |
| 7. | SEQ ID NO: 7 - PAM |
| | NNNNCGAA |
| | (N represents any nucleotide selected from adenine (A), thymine (T), |
| | guanine (G), or cytosine (C)) |
| 8. | SEQ ID NO: 8 - PAM |
| | NNNNCCAA |
| | (N represents any nucleotide selected from adenine (A), thymine (T), |
| | guanine (G), or cytosine (C)) |
| 9. | SEQ ID NO: 9 - PAM |
| | NNNAC |
| | (N represents any nucleotide selected from adenine (A), thymine (T), |
| | guanine (G), or cytosine (C)) |
| 10. | SEQ ID NO: 10 - Forward primer VEGFA bisulfite sequencing in HEK293 |
| | cell line |
| | AGGGGAAATAAGGGATATTTTTTTTA |
| 11. | SEQ ID NO: 11 - Reverse primer VEGFA bisulfite sequencing in HEK293 |
| | cell line |
| | CCAAAATCTCCTCCTAAATTTTTAC |
| 12. | SEQ ID NO: 12 - Forward primer VEGFA bisulfite sequencing in HCT116 |
| | cell line |
| | GGGGAAATAAGGGATATTTTTTTTA |
| 13. | SEQ ID NO: 13 - Reverse primer VEGFA bisulfite sequencing in HCT116 |
| | cell line |
| | AACCCCCAACATCTAATTAATCTTTA |
| 14. | SEQ ID NO: 14 - Forward primer EMX1 bisulfite sequencing |
| | TTGTTTTTTTGGGAGGGAGAT |
| 15. | SEQ ID NO: 15 - Reverse primer EMX1 bisulfite sequencing |
| | CATAAAATCCTTAATAACCAAAAAC |
| 16. | SEQ ID NO: 16 - Forward primer PRDX4 bisulfite sequencing |
| | GGTTGGAGTTGTGTAGGGTTG |
| 17. | SEQ ID NO: 17 - Reverse primer PRDX4 bisulfite sequencing |
| | CCTCTTCTAAAATCTAATCAATAAAAACC |
| 18. | SEQ ID NO: 18 - Forward primer VEGFA |
| | GGAAGGGAGGAGAGATCCCA |
| 19. | SEQ ID NO: 19 - Reverse primer VEGFA |
| | AGCGGGAGGAGTTGTACTCT |
| 20. | SEQ ID NO: 20 - Forward primer EMX1 |
| | AGCGGGAGGAGTTGTACTCT |
| 21. | SEQ ID NO: 21 - Reverse primer EMX1 |
| | GGGGGAGGTGAGGGGTG |
| 22. | SEQ ID NO: 22 - Forward primer PRDX4 |
| | ATGCAAGCCATTCACAAGCA |
| 23. | SEQ ID NO: 23 - Reverse primer PRDX4 |
| | ATGCAAACTCGGGACTAGGG |
| 24. | SEQ ID NO: 24 - ThermoCas9 sgRNA in-vitro transcription |
| | template strand |
| | AAAACGCCTAAGAGTGGGGAATGCCCGAAGAAAGCGGGCGATAGGCGATCCCCAACGCC |
| | ACGGGTCAGTCTGCCTATAGGCAGAAAGCCCTTATCATAGTAACCCTGTTTCCAGGGGAA |
| | CTATGACTACCAGGATCTTGCCATCCTACCTATAGTGAGTCGTATTA |
| 25. | SEQ ID NO: 25 - T7 promoter oligo |
| | TAATACGACTCACTATA |
| 26. | SEQ ID NO: 26 - PAM CC target strand |
| | AGCTTCCTGTATACCAGGATCTTGCCATCCTACCTCTAGA |
| 27. | SEQ ID NO: 27 - PAM CC non-target strand |
| | TCTAGAGGTAGGATGGCAAGATCCTGGTATACACCAAGCT |
| 28. | SEQ ID NO: 28 - PAM CG target strand |
| | AGCTTCGTGTATACCAGGATCTTGCCATCCTACCTCTAGA |
| 29. | SEQ ID NO: 29 - PAM CG non-target strand |
| | TCTAGAGGTAGGATGGCAAGATCCTGGTATACACGAAGCT |
| 30. | SEQ ID NO: 30 - Methylated PAM CG target strand |
| | AGCTT[5Me~dC]GTGTATACCAGGATCTTGCCATCCTACCTCTAGA |
| | (5Me~dC represents a 5′ methylated cytosine) |
| 31. | SEQ ID NO: 31 - Methylated PAM CG non-target strand |
| | TCTAGAGGTAGGATGGCAAGATCCTGGTATACA[5Me~dC]GAAGCT |
| | (5Me~dC represents a 5′ methylated cytosine) |
| 32. | SEQ ID NO: 32 - HEX-labelled PAM CG target strand |
| | [5~HEX]AGCTTCGTGTATACCAGGATCTTGCCATCCTACCTCTAGA |
| | (5~HEX represents Hexachlorofluorescein) |
| 33. | SEQ ID NO: 33 - HEX-labelled methylated PAM CG target strand |
| | [5~HEX]AGCTT[5Me~dC]GTGTATACCAGGATCTTGCCATCCTACCTCTAGA |
| | (5~HEX represents Hexachlorofluorescein; 5Me~dC represents a |
| | 5′ methylated cytosine) |
| 34. | SEQ ID NO: 34 - FAM-labelled PAM CC non-target strand |
| | [6~FAM]TCTAGAGGTAGGATGGCAAGATCCTGGTATACACCAAGCT |
| | (6~FAM represents fluorescein) |
| 35. | SEQ ID NO: 35 - FAM-labelled PAM CG non-target strand |
| | [6~FAM]TCTAGAGGTAGGATGGCAAGATCCTGGTATACACGAAGCT |
| | (6~FAM represents fluorescein) |
| 36. | SEQ ID NO: 36 - FAM-labelled methylated PAM CG non-target strand |
| | [6~FAM]TCTAGAGGTAGGATGGCAAGATCCTGGTATACA[5Me~dC]GAAGCT |
| | (6~FAM represents fluorescein; 5Me~dC represents a 5′ methylated |
| | cytosine) |
| 37. | SEQ ID NO: 37 - K771A-F |
| | GAATTTTAACGCAAACCGGGAAGAATCGAATTTG |
| 38. | SEQ ID NO: 38 - K711A-R |
| | CAACGGCTGCGTAAATGG |
| 39. | SEQ ID NO: 39 - D723A-R |
| | ATGCAAATTCGATTCTTCCCGG |
| 40. | SEQ ID NO: 40 - D1017A-F |
| | CAAACCATCGCCTCCTCCAATG |
| 41. | SEQ ID NO: 41 - D1017A-R |
| | ATAATAGGCGAACAGATC |
| 42. | SEQ ID NO: 42 - S1019A-F |
| | CATCGACTCCGCCAATGGAGG |
| 43. | SEQ ID NO: 43 - S1019A-R |
| | GTTTGATAATAGGCGAACAGATC |
| 44. | SEQ ID NO: 44 - T9 gRNA target |
| | ACCGATCCCCTGAGAGGACAGGGAACC |
| 45. | SEQ ID NO: 45 - T9 gRNA reverse complement |
| | GACGGTTCCCTGTCCTCTCAGGGGATC |
| 46. | SEQ ID NO: 46 - T3 gRNA target |
| | ACCGAGAGACCTGAACAGCGGAGAGTC |
| 47. | SEQ ID NO: 47 - T3 gRNA reverse complement |
| | GACGACTCTCCGCTGTTCAGGTCTCTC |
| 48. | SEQ ID NO: 48 - T4 gRNA target |
| | ACCGAATGGAGAGGGTCCCGGTGCTGG |
| 49. | SEQ ID NO: 49 - T4 gRNA reverse complement |
| | GACCCAGCACCGGGACCCTCTCCATTC |
| 50. | SEQ ID NO: 50 - T5 gRNA target |
| | ACCGGAGACAGAGGAGAGGCCCCGGA |
| 51. | SEQ ID NO: 51 - T5 gRNA reverse complement |
| | GACTCCGGGGCCTCTCCTCTGTCTCC |
| 52. | SEQ ID NO: 52 - ThermoCas9 |
| | MKYKIGLDIGITSIGWAVINLDIPRIEDLGVRIFDRAENPKTGESLALPRRLARSARRRLRRRKH |
| | RLERIRRLFVREGILTKEELNKLFEKKHEIDVWQLRVEALDRKLNNDELARILLHLAKRRGFRS |
| | NRKSVARDDLEREIKLIFAKQREYGNIVCTEAFEHEYISIWASQRPFASKDDIEKKVGFCTFEPK |
| | EKRAPKATYTFQSFTVWEHINKLRLVSPGGIRALTDDERRLIYKQAFHKNKITFHDVRTLLNLP |
| | DDTRFKGLLYDRNTTLKENEKVRFLELGAYHKIRKAIDSVYGKGAAKSFRPIDFDTFGYALTM |
| | FKDDTDIRSYLRNEYEQNGKRMENLADKVYDEELIEELLNLSFSKFGHLSLKALRNILPYMEQ |
| | GEVYSTACERAGYTFTGPKKKQKTVLLPNIPPIANPVVMRALTQARKVVNAIIKKYGSPVSIHI |
| | ELARELSQSFDERRKMQKEQEGNRKKNETAIRQLVEYGLTLNPTGLDIVKFKLWSEQNGKCA |
| | YSLQPIEIERLLEPGYTEVDHVIPYSRSLDDSYTNKVLVLTKENREKGNRTPAEYLGLGSERWQ |
| | QFETFVLTNKQFSKKKRDRLLRLHYDENEENEFKNRNLNDTRYISRFLANFIREHLKFADSDD |
| | KQKVYTVNGRITAHLRSRWNFNKNREESNLHHAVDAAIVACTTPSDIARVTAFYQRREQNKE |
| | LSKKTDPQFPQPWPHFADELQARLSKNPKESIKALNLGNYDNEKLESLQPVFVSRMPKRSITG |
| | AAHQETLRRYIGIDERSGKIQTVVKKKLSEIQLDKTGHFPMYGKESDPRTYEAIRQRLLEHNN |
| | DPKKAFQEPLYKPKKNGELGPIIRTIKIIDTTNQVIPLNDGKTVAYNSNIVRVDVFEKDGKYYC |
| | VPIYTIDMMKGILPNKAIEPNKPYSEWKEMTEDYTFRFSLYPNDLIRIEFPREKTIKTAVGEEIKI |
| | KDLFAYYQTIDSSNGGLSLVSHDNNFSLRSIGSRTLKRFEKYQVDVLGNIYKVRGEKRVGVAS |
| | S |
| 53. | SEQ ID NO: 53 - D723A-R |
| | CATGCCGTCGCTGCTGCCATC |
| 54. | SEQ ID NO: 54 |
| | GTCGAA |
| 55. | SEQ ID NO: 55 - EMX1 T4 Guide |
| | AATGGAGAGGGTCCCGGTGCTGG |
| 56. | SEQ ID NO: 56 |
| | GTTGAA |
| 57. | SEQ ID NO: 57 - EMX1 T4 PAM |
| | GCGCCGAA |
| 58. | SEQ ID NO: 58 |
| | TACGAA |
| 59. | SEQ ID NO: 59 - VEGFA T3 Guide |
| | AGAGACCTGAACAGCGGAGAGTC |
| 60. | SEQ ID NO: 60 - VEGFA T3 PAM |
| | CTCACGAA |
| 61. | SEQ ID NO: 61 |
| | TTCACA |
| 62. | SEQ ID NO: 62 - PRDX4 T5 (reverse complement) Guide |
| | GGAGACAGAGGAGAGGCCCCGGA |
| 63. | SEQ ID NO: 63 |
| | TTCGCG |
| 64. | SEQ ID NO: 64 - PRDX4 T5 (reverse complement) |
| | CTCGCGAA |
| 65. | SEQ ID NO: 65 - Nucleic Acid Sequence of FIG. 3B |
| | CTAGAGGTAGGATGGCAAGATCCTGGTATACACCAAGCT |
| 66. | SEQ ID NO: 66 - Nucleic Acid Sequence of FIG. 3B |
| | GATCTCCATCCTACCGTTCTAGGACCATATCTGGTTCGA |
| 67. | SEQ ID NO: 67 - Secondary structures of the observed R-loop structure |
| | GGUAUUAUGGCAAGAUCCUGGUAGUCAUAGUUCCCCUGGAAACAGGGUUACUAUGAUA |
| | AGGGCUUUCUGCCUAUAGGCAGACUGACCCGUGGCGUUGGGGAUCGCCUAUUCCCCAC |
| | UCUGGGCGU |
| 68. | SEQ ID NO: 68 - ThermoCas9 HNH Catalytic Site |
| | ----TEVDHVIPYSRSLD----ENREKGNRT---- |
| | Dashed lines indicate close contact distances. |
| 69. | SEQ ID NO: 69 - AnCas9 HNH catalytic site |
| | ---CQLDHIVPQAGPGS----CNRSKSNTP--- |
| | Dashed lines indicate close contact distances. |
| 70. | SEQ ID NO: 70 - AceCas9 HNH Catalytic Site |
| | ---SELDHIVPRTDGGS----CNKEKGRRP--- |
| | Dashed lines indicate close contact distances. |
| 71. | SEQ ID NO: 71 - SpCas9 HNH Catalytic Site |
| | ----YDVDHIVPQSFLKD----KNRGKSDNV--- |
| | Dashed lines indicate close contact distances. |
| 72. | SEQ ID NO: 72 - CjCas9 HNH Catalytic Site |
| | ----LEIDHIYPYSRSFD----QNQEKLNQT---- |
| | Dashed lines indicate close contact distances. |
| 73. | SEQ ID NO: 73 - Nm1Cas9 HNH Catalytic Site |
| | ----VEIDHALPFSRTWD----ENQNKGNQT---- |
| | Dashed lines indicate close contact distances. |
| 74. | SEQ ID NO: 74 - HpaCas9 HNH Catalytic Site |
| | ----VEVDHALPFSRTWD----ENQNKGNLT---- |
| | Dashed lines indicate close contact distances. |
| 75. | SEQ ID NO: 75 - StCas9 HNH Catalytic Site |
| | ----FEVDHILPLSITFD----ANQEKGQRT---- |
| | Dashed lines indicate close contact distances. |
| 76. | SEQ ID NO: 76 - SaCas9 HNH Catalytic Site |
| | ---YEVDHIIPRSVSFD----ENSKKGNRT--- |
| | Dashed lines indicate close contact distances. |
| 77. | SEQ ID NO: 77 - ThermoCas9 RuvC catalytic site |
| | MKYKIGLDIGITS------------HIELAR----KNREESNLHHAVDAAIVA---- |
| | Dashed lines indicate close contact distances. |
| 78. | SEQ ID NO: 78 - AnCas9 RuvC catalytic site |
| | RVGIDVGTHS------------HVEHVR----KDRI-DRRHHAVDASVVA---- |
| | Dashed lines indicate close contact distances. |
| 79. | SEQ ID NO: 79 - AceCas9 RuvC catalytic site |
| | RLGVDVGERS------------VVELAR----K-RL-DRRHHAVDAVVLT---- |
| | Dashed lines indicate close contact distances. |
| 80. | SEQ ID NO: 80 - SpCas9 RuvC catalytic site |
| | MDKKYSIGLDIGTNS------------VIEMAR----KVREINNYHHAHDAYLNA---- |
| | Dashed lines indicate close contact distances. |
| 81. | SEQ ID NO: 81 - CjCas 9 RuvC catalytic site |
| | MARILAFDIGISS------------NIELAR----KDR-NNHLHHAIDAVIIA---- |
| | Dashed lines indicate close contact distances. |
| 82. | SEQ ID NO: 82 - Nm1Cas9 RuvC catalytic site |
| | ILGLDIGIAS------------HIETAR----KVRAENDRHHALDAVVVA---- |
| | Dashed lines indicate close contact distances. |
| 83. | SEQ ID NO: 83 - HpaCas9 RuvC catalytic site |
| | KNLNYILGLDLGIAS------------HIETGR----KSREDNDRHHALDAVVVA---- |
| | Dashed lines indicate close contact distances. |
| 84. | SEQ ID NO: 84 - StCas9 RuvC catalytic site |
| | MSDLVLGLDIGIGS------------VIEMAR----KTRD-TYHHHAVDALIIA---- |
| | Dashed lines indicate close contact distances. |
| 85. | SEQ ID NO: 85 - SaCas9 RuvC catalytic site |
| | MKRNYILGLDIGITS------------IIELAR----KERNKGYKHHAEDALIIA---- |
| | Dashed lines indicate close contact distances. |
| 86. | SEQ ID NO: 86 - Indel 0 in FIG. 9A |
| | CACCCATCCCCTGAGAGGACAGGGAACCCCATCCAACAGCCAGGGGGACTCACCTTCGTG |
| | ATGATTCTGCCCTCC |
| 87. | SEQ ID NO: 87 - Indel -1 in FIG. 9A |
| | CACCCATCCCCTGAGAGGACAGGGACCCCATCCAACAGCCAGGGGGACTCACCTTCGTGA |
| | TGATTCTGCCCTCC |
| 88. | SEQ ID NO: 88 - Indel +1 in FIG. 9A |
| | CACCCATCCCCTGAGAGGACAGGGNACCCCATCCAACAGCCAGGGGGACTCACCTTCGTG |
| | ATGATTCTGCCCTC |
| | (N represents any nucleotide selected from adenine (A), thymine |
| | (T), guanine (G), or cytosine (C)) |
| 89. | SEQ ID NO: 89 - Indel -2 in FIG. 9A |
| | CACCCATCCCCTGAGAGGACAGGACCCCATCCAACAGCCAGGGGGACTCACCTTCGTGAT |
| | GATTCTGCCCTCC |
| 90. | SEQ ID NO: 90 - Indel -21 in FIG. 9A |
| | CACCCATCCCCTGAGAGGACAGGGGGACTCACCTTCGTGATGATTCTGCCCTCC |
| 91. | SEQ ID NO: 91 - Indel -1 in FIG. 9A |
| | CACCCATCCCCTGAGAGGACAGGGACCCCATCCAACAGCCAGGGGGACTCACCTTCGTGA |
| | TGATTCTGCCCTCC |
| 92. | SEQ ID NO: 92 - Indel -17 in FIG. 9A |
| | CACCCATCCCCTGAGAGGACAGCCAGGGGGACTCACCTTCGTGATGATTCTGCCCTCC |
| 93. | SEQ ID NO: 93 - Indel -10 in FIG. 9A |
| | CACCCATCCCCTGAGAGGACAGCCAACAGCCAGGGGGACTCACCTTCGTGATGATTCTGC |
| | CCTCC |
| 94. | SEQ ID NO: 94 - Indel -7 in FIG. 9A |
| | CACCCATCCCCTGAGAGGACAGCATCCAACAGCCAGGGGGACTCACCTTCGTGATGATTC |
| | TGCCCTCC |
| 95. | SEQ ID NO: 95 - Indel -21 in FIG. 9A |
| | CACCCATCCCCTGAGAGGACAGGGGGACTCACCTTCGTGATGATTCTGCCCTCC |
| 96. | SEQ ID NO: 96- Indel -17 in FIG. 9A |
| | CACCCATCCCCTGAGAGGACAGGCAGGGGGACTCACCTTCGTGATGATTCTGCCCTCC |
| 97. | SEQ ID NO: 97 - Indel -10 in FIG. 9A |
| | CACCCATCCCCTGAGAGGACAGGCAACAGCCAGGGGGACTCACCTTCGTGATGATTCTGC |
| | CCTCC |
| 98. | SEQ ID NO: 98 - Indel -3 in FIG. 9A |
| | CACCCATCCCCTGAGAGGACAGGCCCCATCCAACAGCCAGGGGGACTCACCTTCGTGATG |
| | ATTCTGCCCTCC |
| 99. | SEQ ID NO: 99 - Indel -2 in FIG. 9A |
| | CACCCATCCCCTGAGAGGACAGGGACCCATCCAACAGCCAGGGGGACTCACCTTCGTGAT |
| | GATTCTGCCTCC |
| 100. | SEQ ID NO: 100 - Indel 0 in FIG. 9B |
| | CACCCATCCCCTGAGAGGACAGGGAACCCCATCCAACAGCCAGGGGGACTCACCTTCGTG |
| | ATGATTCTGCCCTCC |
| 101. | SEQ ID NO: 101 - Indel -1 in FIG. 9B |
| | CACCCATCCCCTGAGAGGACAGGGACCCCATCCAACAGCCAGGGGGACTCACCTTCGTGA |
| | TGATTCTGCCCTCC |
| 102. | SEQ ID NO: 102 - Indel +1 in FIG. 9B |
| | CACCCATCCCCTGAGAGGACAGGGANACCCCATCCAACAGCCAGGGGGACTCACCTTCGT |
| | GATGATTCTGCCCTC |
| 103. | SEQ ID NO: 103 - Indel -17 in FIG. 9B |
| | CACCCATCCCCTGAGAGGACAGCCAGGGGGACTCACCTTCGTGATGATTCTGCCCTCC |
| 104. | SEQ ID NO: 104 - Indel -1 in FIG. 9B |
| | CACCCATCCCCTGAGAGGACAGGGACCCCATCCAACAGCCAGGGGGACTCACCTTCGTGA |
| | TGATTCTGCCCTCC |
| 105. | SEQ ID NO: 105 - Indel -2 in FIG. 9B |
| | CACCCATCCCCTGAGAGGACAGGACCCCATCCAACAGCCAGGGGGACTCACCTTCGTGAT |
| | GATTCTGCCCTCC |
| 106. | SEQ ID NO: 106 - Indel -17 in FIG. 9B |
| | CACCCATCCCCTGAGAGGACAGCCAGGGGGACTCACCTTCGTGATGATTCTGCCCTCC |
| 107. | SEQ ID NO: 107 - Indel -10 in FIG. 9B |
| | CACCCATCCCCTGAGAGGACATCCAACAGCCAGGGGGACTCACCTTCGTGATGATTCTGC |
| | CCTCC |
| 108. | SEQ ID NO: 108 - Indel -10 in FIG. 9B |
| | CACCCATCCCCTGAGAGGACATCCAACAGCCAGGGGGACTCACCTTCGTGATGATTCTGC |
| | CCTCC |
| 109. | SEQ ID NO: 109 - Indel -18 in FIG. 9B |
| | CACCCATCCCCTGAGAGGACAGCAGGGGGACTCACCTTCGTGATGATTCTGCCCTCC |
| 110. | SEQ ID NO: 110 - Indel -6 in FIG. 9B |
| | CACCCATCCCCTGAGAGGACAGCCATCCAACAGCCAGGGGGACTCACCTTCGTGATGATT |
| | CTGCCCTCC |
| 111. | SEQ ID NO: 111 - Indel 0 in FIG. 9C |
| | CACCCATCCCCTGAGAGGACAGGGAACCCCATCCAACAGCCAGGGGGACTCACCTTCGTG |
| | ATGATTCTGCCCTCC |
| 112. | SEQ ID NO: 112 - Indel -1 in FIG. 9C |
| | CACCCATCCCCTGAGAGGACAGGGACCCCATCCAACAGCCAGGGGGACTCACCTTCGTGA |
| | TGATTCTGCCCTCC |
| 113. | SEQ ID NO: 113 - Indel -17 in FIG. 9C |
| | CACCCATCCCCTGAGAGGACAGGCAGGGGGACTCACCTTCGTGATGATTCTGCCCTCC |
| 114. | SEQ ID NO: 114 - Indel -1 in FIG. 9C |
| | CACCCATCCCCTGAGAGGACAGGGACCCCATCCAACAGCCAGGGGGACTCACCTTCGTGA |
| | TGATTCTGCCCTCC |
| 115. | SEQ ID NO: 115 - Indel +1 in FIG. 9C |
| | CACCCATCCCCTGAGAGGACAGGGANACCCCATCCAACAGCCAGGGGGACTCACCTTCGT |
| | GATGATTCTGCCCTC |
| 116. | SEQ ID NO: 116 - Indel -17 in FIG. 9C |
| | CACCCATCCCCTGAGAGGACAGCCAGGGGGACTCACCTTCGTGATGATTCTGCCCTCC |
| 117. | SEQ ID NO: 117 - Indel -3 in FIG. 9C |
| | CACCCATCCCCTGAGAGGACAGGCCCCATCCAACAGCCAGGGGGACTCACCTTCGTGATG |
| | ATTCTGCCCTCC |
| 118. | SEQ ID NO: 118 - Indel -21 in FIG. 9C |
| | CACCCATCCCCTGAGAGGACAGGGGGACTCACCTTCGTGATGATTCTGCCCTCC |
| 119. | SEQ ID NO: 119 - Indel -10 in FIG. 9C |
| | CACCCATCCCCTGAGAGGACAGCCAACAGCCAGGGGGACTCACCTTCGTGATGATTCTGC |
| | CCTCC |
| 120. | SEQ ID NO: 120 - Indel -7 in FIG. 9C |
| | CACCCATCCCCTGAGAGGACAGCATCCAACAGCCAGGGGGACTCACCTTCGTGATGATTC |
| | TGCCCTCC |
| 121. | SEQ ID NO: 121 - Indel - 10 in FIG. 9C |
| | CACCCATCCCCTGAGAGGACAGCAACAGCCAGGGGGACTCACCTTCGTGATGATTCTGCC |
| | CTCC |
| 122. | SEQ ID NO: 122 - Indel -7 in FIG. 9C |
| | CACCCATCCCCTGAGAGGACAGATCCAACAGCCAGGGGGACTCACCTTCGTGATGATTCT |
| | GCCCTCC |
| 123. | SEQ ID NO: 123 - T3R1, T3R2, T3R3 Indel 0 in FIG. 9D |
| | CTAGCAGAGACCTGAACAGCGGAGAGTCCTCACGAAACTGAGGGTGAACCTCGTGGTGC |
| | CCAGCTCTTTCTTTCT |
| 124. | SEQ ID NO: 124 - T4R1 Indel 0 in FIG. 9D |
| | GTAGAAATGGAGAGGGTCCCGGTGCTGGGCGCCGAAGAAGGAGTGCGGGGGCTGCAGCG |
| | GGGAGGCGCCCAGCTG |
| 125. | SEQ ID NO: 125 - T4R1 Indel -7 in FIG. 9D |
| | GTAGAAATGGAGAGGGTCCCGGTGCCGAAGAAGGAGTGCGGGGGCTGCAGCGGGGAGGC |
| | GCCCAGCTG |
| 126. | SEQ ID NO: 126 - T4R1 Indel -1 in FIG. 9D |
| | GTAGAAATGGAGAGGGTCCCGGTGCNTGGGCGCCGAAGAAGGAGTGCGGGGGCTGCAGC |
| | GGGGAGGCGCCCAGCT |
| 127. | SEQ ID NO: 127 - T4R1 Indel -7 in FIG. 9D |
| | GTAGAAATGGAGAGGGTCCCGGCGCCGAAGAAGGAGTGCGGGGGCTGCAGCGGGGAGG |
| | CGCCCAGCTG |
| 128. | SEQ ID NO: 128 - T4R1 Indel -7 in FIG. 9D |
| | GTAGAAATGGAGAGGGTCCCGGTGCCGAAGAAGGAGTGCGGGGGCTGCAGCGGGGAGGC |
| | GCCCAGCTG |
| 129. | SEQ ID NO: 129 - T4R2 Indel 0 in FIG. 9D |
| | GTAGAAATGGAGAGGGTCCCGGTGCTGGGCGCCGAAGAAGGAGTGCGGGGGCTGCAGCG |
| | GGGAGGCGCCCAGCTG |
| 130. | SEQ ID NO: 130 - T4R2 Indel -7 in FIG. 9D |
| | GTAGAAATGGAGAGGGTCCCGGTGCCGAAGAAGGAGTGCGGGGGCTGCAGCGGGGAGGC |
| | GCCCAGCTG |
| 131. | SEQ ID NO: 131 - T4R2 Indel +1 in FIG. 9D |
| | GTAGAAATGGAGAGGGTCCCGGTGCNTGGGCGCCGAAGAGGAGTGCGGGGGCTGCAGCG |
| | GGGAGGCGCCCAGCT |
| 132. | SEQ ID NO: 132 - T4R2 Indel -7 in FIG. 9D |
| | GTAGAAATGGAGAGGGTCCCGGCGCCGAAGAAGGAGTGCGGGGGCTGCAGCGGGGAGG |
| | CGCCCAGCTG |
| 133. | SEQ ID NO: 133 - T4R3 Indel 0 in FIG. 9D |
| | GTAGAAATGGAGAGGGTCCCGGTGCTGGGCGCCGAAGAAGGAGTGCGGGGGCTGCAGCG |
| | GGGAGGCGCCCAGCTG |
| 134. | SEQ ID NO: 134 - T4R3 Indel -7 in FIG. 9D |
| | TGCCGAAGAAGGAGTGCGGGGGCTGCAGCGGGGAGGCGCCCAGCTG |
| 135. | SEQ ID NO: 135 - T4R3 Indel +1 in FIG. 9D |
| | TGCNTGGGCGCCGAAGAAGGAGTGCGGGGGCTGCAGCGGGGAGGCGCCCAGCT |
| | (N represents any nucleotide selected from adenine |
| | (A), thymine (T), guanine (G), or cytosine (C); R |
| | represents a purine nucleotide) |
| 136. | SEQ ID NO: 136 - T4R3 Indel -7 in FIG. 9D |
| | CGCCGAAGAAGGAGTGCGGGGGCTGCAGCGGGGAGGCGCCCAGCTG |
| 137. | SEQ ID NO: 137 - T5R1, T5R2, T5R4 Indel 0 in FIG. 9D |
| | GGCTGGGAGACAGAGGAGAGGCCCCGGACTCGCGAAGAGGAGTGCCACTTCTACGCGGG |
| | TGGACAAGTGTACCCG |
| 138. | SEQ ID NO: 138 - T9R1, T9R3 Indel 0 in FIG. 10A |
| | CACCCATCCCCTGAGAGGACAGGGAACCCCATCCAACAGCCAGGGGGACTCACCTTCGTG |
| | ATGATTCTGCCCTCC |
| 139. | SEQ ID NO: 139 - T9R1, T9R3 Indel -1 in FIG. 10A |
| | CACCCATCCCCTGAGAGGACAGGGACCCCATCCAACAGCCAGGGGGACTCACCTTCGTGA |
| | TGATTCTGCCCTCC |
| 140. | SEQ ID NO: 140 - T9R1, T9R3 Indel +1 in FIG. 10A |
| | CACCCATCCCCTGAGAGGACAGGGANACCCCATCCAACAGCCAGGGGGACTCACCTTCGT |
| | GATGATTCTGCCCTC |
| 141. | SEQ ID NO: 141 - T9R2 Indel 0 in FIG. 10A |
| | CACCCATCCCCTGAGAGGACAGGGAACCCCATCCAACAGCCAGGGGGACTCACCTTCGTG |
| | ATGATTCTGCCCTCC |
| 142. | SEQ ID NO: 142 - T9R2 Indel -1 in FIG. 10A |
| | CACCCATCCCCTGAGAGGACAGGGACCCCATCCAACAGCCAGGGGGACTCACCTTCGTGA |
| | TGATTCTGCCCTCC |
| 143. | SEQ ID NO: 143 - T9R2 Indel +1 in FIG. 10A |
| | CACCCATCCCCTGAGAGGACAGGGANACCCCATCCAACAGCCAGGGGGACTCACCTTCGT |
| | GATGATTCTGCCCTC |
| 144. | SEQ ID NO: 144 - T9R2 Indel -9 in FIG. 10A |
| | CACCCATCCCCTGAGAGGACAGGGACAACAGCCAGGGGGACTCACCTTCGTGATGATTCT |
| | GCCCTCC |
| 145. | SEQ ID NO: 145 - T9R2 Indel -7 in FIG. 10A |
| | CACCCATCCCCTGAGAGGACAGGGACCAACAGCCAGGGGGACTCACCTTCGTGATGATTC |
| | TGCCCTCC |
| 146. | SEQ ID NO: 146 - T9R2 Indel -3 in FIG. 10A |
| | CACCCATCCCCTGAGAGGACAGGGACCATCCAACAGCCAGGGGGACTCACCTTCGTGATG |
| | ATTCTGCCCTCC |
| 147. | SEQ ID NO: 147 - T9R2 Indel -17 in FIG. 10A |
| | CACCCATCCCCTGAGAGGACAGGCAGGGGGACTCACCTTCGTGATGATTCTGCCCTCC |
| 148. | SEQ ID NO: 148 - T3R1, T3R2, T3R3 Indel 0 in FIG. 10B |
| | CTAGCAGAGACCTGAACAGCGGAGAGTCCTCACGAAACTGAGGGTGAACCTCGTGGTGC |
| | CCAGCTCTTTCTTTCT |
| 149. | SEQ ID NO: 149 - T4R1, T4R2, T4R3 Indel 0 in FIG. 10B |
| | CTAGAAATGGAGAGGGTCCCGGTCTGGGCGCCGAAGAAGGAGTGCGGGGGCTGCAGCGG |
| | GGAGGCGCCCAGCTG |
| 150. | SEQ ID NO: 150 - T5R1 Indel 0 in FIG. 10C |
| | GGCTGGGAGACAGAGGAGAGGCCCCGGACTCGCGAAGAGGAGTGCCACTTCTACGCGGG |
| | TGGACAAGTGTACCCG |
| 151. | SEQ ID NO: 151 - T5R1 Indel -4 in FIG. 10C |
| | GGCTGGGAGACAGAGGAGAGGCCACTCGCGAAGAGGAGTGCCACTTCTACGCGGGTGGA |
| | CAAGTGTACCCG |
| 152. | SEQ ID NO: 152 - T5R1 Indel -14 in FIG. 10C |
| | GGCTGGGAGACAGAGGAGAGGCCAGGAGTGCCACTTCTACGCGGGTGGACAAGTGTACC |
| | CG |
| 153. | SEQ ID NO: 153 - T5R1 Indel -1 in FIG. 10C |
| | GGCTGGGAGACAGAGGAGAGGCCCCGACTCGCGAAGAGGAGTGCCACTTCTACGCGGGT |
| | GGACAAGTGTACCCG |
| 154. | SEQ ID NO: 154 - T5R1 Indel +1 in FIG. 10C |
| | GGCTGGGAGACAGAGGAGAGGCCCCNGGACTCGCGAAGAGGAGTGCCACTTCTACGCGG |
| | GTGGACAAGTGTACCC |
| | (N represents any nucleotide selected from adenine (A), |
| | thymine (T), guanine (G), or cytosine (C); R represents |
| | a purine nucleotide) |
| 155. | SEQ ID NO: 155 - T5R1 Indel -13 in FIG. 10C |
| | GGCTGGGAGACAGAGGAGAGGCCCCGGAGTGCCACTTCTACGCGGGTGGACAAGTGTAC |
| | CCG |
| 156. | SEQ ID NO: 156 - T5R2 Indel 0 in FIG. 10D |
| | GGCTGGGAGACAGAGGAGAGGCCCCGGACTCGCGAAGAGGAGTGCCACTTCTACGCGGG |
| | TGGACAAGTGTACCCG |
| 157. | SEQ ID NO: 157 - T5R2 Indel -2 in FIG. 10D |
| | GGCTGGGAGACAGAGGAGAGGCCCCACTCGCGAAGAGGAGTGCCACTTCTACGCGGGTG |
| | GACAAGTGTACCCG |
| 158. | SEQ ID NO: 158 - T5R2 Indel -11 in FIG. 10D |
| | GGCTGGGAGACAGAGGAGAGGCCCCGAGGAGTGCCACTTCTACGCGGGTGGACAAGTGT |
| | ACCCG |
| 159. | SEQ ID NO: 159 - T5R2 Indel -14 in FIG. 10D |
| | GGCTGGGAGACAGAGGAGAGGCCCCGAGTGCCACTTCTACGCGGGTGGACAAGTGTACC |
| | CG |
| 160. | SEQ ID NO: 160 - T5R2 Indel +20 in FIG. 10D |
| | GGCTGGGAGACAGAGGAGAGGCCCCNNNNNNNNNNNNNNNNNNNNGGACTCGCGAAGA |
| | GGAGTGCCACTTCTACG |
| | (N represents any nucleotide selected from adenine (A), |
| | thymine (T), guanine (G), or cytosine (C); R represents |
| | a purine nucleotide) |
| 161. | SEQ ID NO: 161 - T5R2 Indel +14 in FIG. 10D |
| | GGCTGGGAGACAGAGGAGAGGCCCCNNNNNNNNNNNNNNACTCGCGAAGAGGAGTGCC |
| | ACTTCTACGCGGGTG |
| | (N represents any nucleotide selected from adenine (A), |
| | thymine (T), guanine (G), or cytosine (C); R represents |
| | a purine nucleotide) |
| 162. | SEQ ID NO: 162 - T5R3 Indel 0 in FIG. 10E |
| | GGCTGGGAGACAGAGGAGAGGCCCCGGACTCGCGAAGAGGAGTGCCACTTCTACGCGGG |
| | TGGACAAGTGTACCCG |
| 163. | SEQ ID NO: 163 - T5R3 Indel -1 in FIG. 10E |
| | GGCTGGGAGACAGAGGAGAGGCCCGGACTCGCGAACAGGAGTGCCACTTCTACGCGGGT |
| | GGACAAGTGTACCCG |
| 164. | SEQ ID NO: 164 - T5R3 Indel -1 in FIG. 10E |
| | GGCTGGGAGACAGAGGAGAGGCCCCGACTCGCGAAGAGGAGAGCCACTTCTACGCGGGT |
| | GGACAAGTGTACCCG |
| 165. | SEQ ID NO: 165 - T5R3 Indel -17 in FIG. 10E |
| | GGCTGGGAGACAGAGGAGAGAGGAGTGCCACTTCTACGCGGGTGGACAAGTGTACCCG |
| 166. | SEQ ID NO: 166 - T5R3 Indel -17 in FIG. 10E |
| | GGCTGGGAGACAGAGGAGAGGAGTGCCACTTCTACGCGGTGGACAAGTGTACCCG |
| 167. | SEQ ID NO: 167 - T5R3 Indel -21 in FIG. 10E |
| | GGCTGGGAGACAGAGGAGAGGCGCCACTTCTACGCGGGTGGACAAGTGTACCCG |
| 168. | SEQ ID NO: 168 - T5R3 Indel -6 in FIG. 10E |
| | GGCTGGGAGACAGAGGAGAGGCCTCGCGAAGAGGAGTGCCACTTCTACGCGGGTGGACA |
| | AGTGTACCCG |
| 169. | SEQ ID NO: 169 - T5R3 Indel -12 in FIG. 10E |
| | GGCTGGGAGACAGAGGAGAGGGGAGTGCCACTTCTACGCGGGTGGACAAGTGTACCCG |
| 170. | SEQ ID NO: 170 - T5R3 Indel -6 in FIG. 10E |
| | GGCTGGGAGACAGAGGAGAGGCCTCGCGAAGAGGAGTGCCACTTCTACGCGGGTGGACA |
| | AGTGTACCCG |
| 171. | SEQ ID NO: 171 - Nucleic acid in FIG. 12 |
| | CCTGGTATATACCAGG |
| 172. | SEQ ID NO: 172 - sgRNA in FIG. 12 |
| | CCUGGUAG |
| 173. | SEQ ID NO: 173 - PAM nucleotide in FIG. 5A |
| | TACACCAA |
| 174. | SEQ ID NO: 174 - PAM nucleotide in FIG. 5A |
| | ATGTGGTT |

### REFERENCES

ADDIN EN.REFLIST 1 Wang, J. Y. & Doudna, J. A. CRISPR technology: A decade of genome editing is only the beginning. Science 379, eadd8643, doi: 10.1126/science.add8643 (2023).
2 Zhang, F. Development of CRISPR-Cas systems for genome editing and beyond. Quarterly reviews of biophysics 52, doi: ARTN e6 10.1017/S0033583519000052 (2019).
3 Charpentier, E. CRISPR-Cas9: how research on a bacterial RNA-guided mechanism opened new perspectives in biotechnology and biomedicine. EMBO Mol Med 7, 363-365, doi: 10.15252/emmm.201504847 (2015).
4 Tsui, T. K. & Li, H. Structure Principles of CRISPR-Cas Surveillance and Effector Complexes. Annu Rev Biophys, doi: 10.1146/annurev-biophys-060414-033939 (2015).
5 Nunez, J. K., Harrington, L. B. & Doudna, J. A. Chemical and Biophysical Modulation of Cas9 for Tunable Genome Engineering. ACS Chem Biol, doi: 10.1021/acschembio.5b01019 (2016).
6 Garcia-Doval, C. & Jinek, M. Molecular architectures and mechanisms of Class 2 CRISPR-associated nucleases. Current opinion in structural biology 47, 157-166, doi: 10.1016/j.sbi.2017.10.015 (2017).
7 Law, J. A. & Jacobsen, S. E. Establishing, maintaining and modifying DNA methylation patterns in plants and animals. Nat Rev Genet 11, 204-220, doi: 10.1038/nrg2719 (2010).
8 Ziller, M. J. et al. Charting a dynamic DNA methylation landscape of the human genome. Nature 500, 477-481, doi: 10.1038/nature12433 (2013).
9 Bird, A. DNA methylation patterns and epigenetic memory. Genes Dev 16, 6-21, doi: 10.1101/gad.947102 (2002).
10 Greenberg, M. V. C. & Bourc′his, D. The diverse roles of DNA methylation in mammalian development and disease. Nature reviews. Molecular cell biology 20, 590-607, doi: 10.1038/s41580-019-0159-6 (2019).
11 Krepelova, A. & Neri, F. DNA methylation controls hematopoietic stem cell aging. Nat Aging, doi: 10.1038/s43587-023-00511-0 (2023).
12 Jones, P. A., Issa, J. P. & Baylin, S. Targeting the cancer epigenome for therapy. Nat Rev Genet 17, 630-641, doi: 10.1038/nrg.2016.93 (2016).
13 Yaung, S. J., Esvelt, K. M. & Church, G. M. CRISPR/Cas9-mediated phage resistance is not impeded by the DNA modifications of phage T4. PLOS One 9, e98811, doi: 10.1371/journal.pone.0098811 (2014).
14 Hsu, P. D. et al. DNA targeting specificity of RNA-guided Cas9 nucleases. Nature biotechnology 31, 827-832, doi: 10.1038/nbt.2647 (2013).
15 Anzalone, A. V., Koblan, L. W. & Liu, D. R. Genome editing with CRISPR-Cas nucleases, base editors, transposases and prime editors. Nature biotechnology 38, 824-844, doi: 10.1038/s41587-020-0561-9 (2020).
16 Loyfer, N. et al. A DNA methylation atlas of normal human cell types. Nature, 1-10, doi: 10.1038/s41586-022-05580-6 (2023).
17 Yousefi, P. D. et al. DNA methylation-based predictors of health: applications and statistical considerations. Nat Rev Genet 23, 369-383, doi: 10.1038/s41576-022-00465-w (2022).
18 Baylin, S. B. & Jones, P. A. A decade of exploring the cancer epigenome-biological and translational implications. Nat Rev Cancer 11, 726-734, doi: 10.1038/nrc3130 (2011).
19 Hanahan, D. Hallmarks of Cancer: New Dimensions. Cancer Discov 12, 31-46, doi: 10.1158/2159-8290.CD-21-1059 (2022).
20 Chemi, F. et al. cfDNA methylome profiling for detection and subtyping of small cell lung cancers. Nat Cancer 3, 1260-1270, doi: 10.1038/s43018-022-00415-9 (2022).
21 Nuzzo, P. V. et al. Detection of renal cell carcinoma using plasma and urine cell-free DNA methylomes. Nat Med 26, 1041-1043, doi: 10.1038/s41591-020-0933-1 (2020).
22 Cohen, J. D. et al. Detection and localization of surgically resectable cancers with a multi-analyte blood test. Science 359, 926-930, doi: 10.1126/science.aar3247 (2018).
23 Liu, X. S. et al. Editing DNA Methylation in the Mammalian Genome. Cell 167, 233-247 e217, doi: 10.1016/j.cell.2016.08.056 (2016).
24 Nakamura, M., Gao, Y., Dominguez, A. A. & Qi, L. S. CRISPR technologies for precise epigenome editing. Nat Cell Biol 23, 11-22, doi: 10.1038/s41556-020-00620-7 (2021).
25 Das, A. et al. The molecular basis for recognition of 5′-NNNCC-3′ PAM and its methylation state by Acidothermus cellulolyticus Cas9. Nature communications 11, 6346, doi: 10.1038/s41467-020-20204-1 (2020).
26 Mougiakos, I. et al. Characterizing a thermostable Cas9 for bacterial genome editing and silencing. Nature communications 8, 1647, doi: 10.1038/s41467-017-01591-4 (2017).
27 Trasanidou, D. et al. Efficient Genome and Base Editing in Human Cells Using ThermoCas9. CRISPR J 6, 278-288, doi: 10.1089/crispr.2023.0005 (2023).
28 Pinney, S. E. Mammalian Non-CpG Methylation: Stem Cells and Beyond. Biology (Basel) 3, 739-751, doi: 10.3390/biology3040739 (2014).
29 Guo, J. U. et al. Distribution, recognition and regulation of non-CpG methylation in the adult mammalian brain. Nat Neurosci 17, 215-222, doi: 10.1038/nn.3607 (2014).
30 Yu, B. et al. Genome-wide, Single-Cell DNA Methylomics Reveals Increased Non-CpG Methylation during Human Oocyte Maturation. Stem Cell Reports 9, 397-407, doi: 10.1016/j.stemcr.2017.05.026 (2017).
31 Patil, V. et al. Human mitochondrial DNA is extensively methylated in a non-CpG context. Nucleic Acids Res 47, 10072-10085, doi: 10.1093/nar/gkz762 (2019).
32 Schmitz, R. J., Lewis, Z. A. & Goll, M. G. DNA Methylation: Shared and Divergent Features across Eukaryotes. Trends in genetics: TIG 35, 818-827, doi: 10.1016/j.tig.2019.07.007 (2019).
33 de Mendoza, A., Lister, R. & Bogdanovic, O. Evolution of DNA Methylome Diversity in Eukaryotes. Journal of molecular biology, doi: 10.1016/j.jmb.2019.11.003 (2019).
34 Jiang, F. & Doudna, J. A. CRISPR-Cas9 Structures and Mechanisms. Annu Rev Biophys 46, 505-529, doi: 10.1146/annurev-biophys-062215-010822 (2017).
35 Cofsky, J. C., Soczek, K. M., Knott, G. J., Nogales, E. & Doudna, J. A. CRISPR-Cas9 bends and twists DNA to read its sequence. Nat Struct Mol Biol 29, 395-402, doi: 10.1038/s41594-022-00756-0 (2022).
36 Pacesa, M. et al. R-loop formation and conformational activation mechanisms of Cas9. Nature 609, 191-196, doi: 10.1038/s41586-022-05114-0 (2022).
37 Sternberg, S. H., LaFrance, B., Kaplan, M. & Doudna, J. A. Conformational control of DNA target cleavage by CRISPR-Cas9. Nature 527, 110-113, doi: 10.1038/nature 15544 (2015).
38 Das, A. et al. Coupled catalytic states and the role of metal coordination in Cas9. Nat Catalysis, 6346 (In press).
39 Nierzwicki, L. et al. Principles of target DNA cleavage and the role of Mg2+ in the catalysis of CRISPR-Cas9. Nat Catal 5, 912-922, doi: 10.1038/s41929-022-00848-6 (2022).
40 Das, A. et al. Coupled catalytic states and the role of metal coordination in Cas9. Nat Catal (2023).
41 Hand, T. H. et al. Catalytically Enhanced Cas9 through Directed Protein Evolution. CRISPR J 4, 223-232, doi: 10.1089/crispr.2020.0092 (2021).
42 Ma, E., Harrington, L. B., O'Connell, M. R., Zhou, K. & Doudna, J. A. Single-Stranded DNA Cleavage by Divergent CRISPR-Cas9 Enzymes. Mol Cell 60, 398-407, doi: 10.1016/j.molcel.2015.10.030 (2015).
43 Mir, A., Edraki, A., Lee, J. & Sontheimer, E. J. Type II-C CRISPR-Cas9 Biology, Mechanism, and Application. ACS Chem Biol 13, 357-365, doi: 10.1021/acschembio.7b00855 (2018).
44 Zhang, K. Gctf: Real-time CTF determination and correction. J Struct Biol 193, 1-12, doi: 10.1016/j.jsb.2015.11.003 (2016).
45 Bepler, T. et al. Positive-unlabeled convolutional neural networks for particle picking in cryo-electron micrographs. Nature methods 16, 1153-1160, doi: 10.1038/s41592-019-0575-8 (2019).
46 Punjani, A., Rubinstein, J. L., Fleet, D. J. & Brubaker, M. A. cryoSPARC: algorithms for rapid unsupervised cryo-EM structure determination. Nature methods 14, 290-296, doi: 10.1038/nmeth.4169 (2017).
47 Zivanov, J. et al. A Bayesian approach to single-particle electron cryo-tomography in RELION-4.0. bioRxiv (2022).
48 Punjani, A., Zhang, H. & Fleet, D. J. Non-uniform refinement: adaptive regularization improves single-particle cryo-EM reconstruction. Nature methods 17, 1214-1221, doi: 10.1038/s41592-020-00990-8 (2020).
49 Emsley, P., Lohkamp, B., Scott, W. G. & Cowtan, K. Features and development of Coot. Acta Crystallogr D Biol Crystallogr 66, 486-501, doi: 10.1107/S0907444910007493 (2010).
50 Liebschner, D. et al. Macromolecular structure determination using X-rays, neutrons and electrons: recent developments in Phenix. Acta Crystallogr D 75, 861-877, doi: 10.1107/S2059798319011471 (2019).
51 Hand, T. H., Das, A. & Li, H. Directed evolution studies of a thermophilic Type II-C Cas9. Methods in enzymology 616, 265-288, doi: 10.1016/bs.mie.2018.10.029 (2019).
52 Consortium, E. P. An integrated encyclopedia of DNA elements in the human genome. Nature 489, 57-74, doi: 10.1038/nature11247 (2012).
53 Luo, Y. et al. New developments on the Encyclopedia of DNA Elements (ENCODE) data portal. Nucleic Acids Res 48, D882-D889, doi: 10.1093/nar/gkz1062 (2020).
54 Robinson, J. T. et al. Integrative genomics viewer. Nature biotechnology 29, 24-26, doi: 10.1038/nbt.1754 (2011).

What is claimed is:

1. A method of detecting one or more methylation sites in a specific location on a target nucleic acid, the method comprising:
a. exposing a target nucleic acid to a Cas9 molecule, wherein the target nucleic acid molecule comprises a protospacer adjacent motif (PAM) recognition sequence which is recognized by the Cas9 molecule, and further wherein the Cas9 molecule cleaves the target nucleic acid at a cleavage site differently upon presence of a methylated cytosine residue within the PAM recognition sequence compared to a non-methylated version of the same cytosine residue; and
b. detecting cleavage of the target nucleic, wherein said cleavage indicates a presence of one or more methylation sites.

2. The method of claim 1, wherein the methylation site within the PAM recognition sequence comprises a cytosine.

3. The method of claim 2, wherein the cytosine is a fifth nucleotide within the PAM.

4. The method of claim 2, wherein the PAM recognition sequence further comprises a guanine.

5. The method of claim 4, wherein the guanine is a sixth nucleotide within the PAM.

6. The method of claim 1, wherein the Cas9 molecule cleaves the target nucleic acid upstream of the PAM sequence.

7. The method of claim 1, wherein the Cas9 molecule is ThermoCas9.

8. The method of claim 7, wherein the ThermoCas9 is naturally occurring.

9. The method of claim 8, wherein the ThermoCas9 comprises SEQ ID NO: 52, or a variation thereof.

10. The method of claim 7, wherein the ThermoCas9 is engineered.

11. The method of claim 3, wherein the PAM recognition site further comprises a second cytosine.

12. The method of claim 11, wherein the second cytosine is a sixth nucleotide within the PAM.

13. The method of claim 1, wherein a site of methylation on the target nucleic acid indicates a higher likelihood of presence of a disease or disorder than a non-methylated version of the target nucleic acid.

14. The method of claim 13, wherein the disease comprises cancer.

15. The method of claim 1, wherein more than one methylation site within the target nucleic acid is recognized by the Cas9 molecule.

16. The method of claim 15, wherein an epigenetic pattern of the target nucleic acid molecule can be established.

17. The method of claim 1, wherein detection occurs by carrying out PCR on non-cleaved target nucleic acid and detecting a product thereof, thereby determining that the target nucleic acid did not comprise one or more methylation sites in a specific location.

18. The method of claim 1, wherein detection occurs via a high throughput assay.

19-56. (canceled)


SEQUENCE LISTING (174 sequences)

SEQ ID NO: 1-9 - skipped (9 entries)

SEQ ID NO: 10 - DNA, 26 bp, synthetic construct
  misc_feature 1..26: note = Description of sequence: Oligonucleotide, specific description: Forward primer VEGFA bisulfite sequencing in HEK293 cell line
  source 1..26: mol_type = other DNA
aggggaaataagggatatttttttta

SEQ ID NO: 11 - DNA, 25 bp, synthetic construct
  misc_feature 1..25: note = Description of sequence: Oligonucleotide, specific description: Reverse primer VEGFA bisulfite sequencing in HEK293 cell line
  source 1..25: mol_type = other DNA
ccaaaatctcctcctaaatttttac

SEQ ID NO: 12 - DNA, 25 bp, synthetic construct
  misc_feature 1..25: note = Description of sequence: Oligonucleotide, specific description: Forward primer VEGFA bisulfite sequencing in HCT116 cell line
  source 1..25: mol_type = other DNA
ggggaaataagggatatttttttta

SEQ ID NO: 13 - DNA, 26 bp, synthetic construct
  misc_feature 1..26: note = Description of sequence: Oligonucleotide, specific description: Reverse primer VEGFA bisulfite sequencing in HCT116 cell line
  source 1..26: mol_type = other DNA
aacccccaacatctaattaatcttta

SEQ ID NO: 14 - DNA, 21 bp, synthetic construct
  misc_feature 1..21: note = Description of sequence: Oligonucleotide, specific description: Forward primer EMX1 bisulfite sequencing
  source 1..21: mol_type = other DNA
ttgtttttttgggagggagat

SEQ ID NO: 15 - DNA, 25 bp, synthetic construct
  misc_feature 1..25: note = Description of sequence: Oligonucleotide, specific description: Reverse primer EMX1 bisulfite sequencing
  source 1..25: mol_type = other DNA
cataaaatccttaataaccaaaaac

SEQ ID NO: 16 - DNA, 21 bp, synthetic construct
  misc_feature 1..21: note = Description of sequence: Oligonucleotide, specific description: Forward primer PRDX4 bisulfite sequencing
  source 1..21: mol_type = other DNA
ggttggagttgtgtagggttg

SEQ ID NO: 17 - DNA, 29 bp, synthetic construct
  misc_feature 1..29: note = Description of sequence: Oligonucleotide, specific description: Reverse primer PRDX4 bisulfite sequencing
  source 1..29: mol_type = other DNA
cctcttctaaaatctaatcaataaaaacc

SEQ ID NO: 18 - DNA, 20 bp, synthetic construct
  misc_feature 1..20: note = Description of sequence: Oligonucleotide, specific description: Forward primer VEGFA
  source 1..20: mol_type = other DNA
ggaagggaggagagatccca

SEQ ID NO: 19 - DNA, 20 bp, synthetic construct
  misc_feature 1..20: note = Description of sequence: Oligonucleotide, specific description: Reverse primer VEGFA
  source 1..20: mol_type = other DNA
agcgggaggagttgtactct

SEQ ID NO: 20 - DNA, 20 bp, synthetic construct
  misc_feature 1..20: note = Description of sequence: Oligonucleotide, specific description: Forward primer EMX1
  source 1..20: mol_type = other DNA
agcgggaggagttgtactct

SEQ ID NO: 21 - DNA, 17 bp, synthetic construct
  misc_feature 1..17: note = Description of sequence: Oligonucleotide, specific description: Reverse primer EMX1
  source 1..17: mol_type = other DNA
gggggaggtgaggggtg

SEQ ID NO: 22 - DNA, 20 bp, synthetic construct
  misc_feature 1..20: note = Description of sequence: Oligonucleotide, specific description: Forward primer PRDX4
  source 1..20: mol_type = other DNA
atgcaagccattcacaagca

SEQ ID NO: 23 - DNA, 20 bp, synthetic construct
  misc_feature 1..20: note = Description of sequence: Oligonucleotide, specific description: Reverse primer PRDX4
  source 1..20: mol_type = other DNA
atgcaaactcgggactaggg

SEQ ID NO: 24 - DNA, 166 bp, synthetic construct
  misc_feature 1..166: note = Description of sequence: Oligonucleotide, specific description: ThermoCas9 sgRNA in-vitro transcription template strand
  source 1..166: mol_type = other DNA
aaaacgcctaagagtggggaatgcccgaagaaagcgggcgataggcgatccccaacgccacgggtcagtctgcctataggcagaaagcccttatcatagtaaccctgtttccaggggaactatgactaccaggatcttgccatcctacctatagtgagtcgtatta

SEQ ID NO: 25 - DNA, 17 bp, synthetic construct
  misc_feature 1..17: note = Description of sequence: Oligonucleotide, specific description: T7 promoter oligo
  source 1..17: mol_type = other DNA
taatacgactcactata

SEQ ID NO: 26 - DNA, 40 bp, synthetic construct
  misc_feature 1..40: note = Description of sequence: Oligonucleotide, specific description: PAM CC target strand
  source 1..40: mol_type = other DNA
agcttcctgtataccaggatcttgccatcctacctctaga

SEQ ID NO: 27 - DNA, 40 bp, synthetic construct
  misc_feature 1..40: note = Description of sequence: Oligonucleotide, specific description: PAM CC non-target strand
  source 1..40: mol_type = other DNA
tctagaggtaggatggcaagatcctggtatacaccaagct

SEQ ID NO: 28 - DNA, 40 bp, synthetic construct
  misc_feature 1..40: note = Description of sequence: Oligonucleotide, specific description: PAM CG target strand
  source 1..40: mol_type = other DNA
agcttcgtgtataccaggatcttgccatcctacctctaga

SEQ ID NO: 29 - DNA, 40 bp, synthetic construct
  misc_feature 1..40: note = Description of sequence: Oligonucleotide, specific description: PAM CG non-target strand
  source 1..40: mol_type = other DNA
tctagaggtaggatggcaagatcctggtatacacgaagct

SEQ ID NO: 30 - DNA, 39 bp, synthetic construct
  misc_feature 1..39: note = Description of sequence: Oligonucleotide, specific description: Methylated a protospacer adjacent motif CG target strand; (5Me˜dC represents a 5 methylated cytosine)
  source 1..39: mol_type = other DNA
  modified_base 6: mod_base = m5c
agcttgtgtataccaggatcttgccatcctacctctaga

SEQ ID NO: 31 - DNA, 40 bp, synthetic construct; identical to SEQ ID NO: 29
  misc_feature 1..40: note = Description of sequence: Oligonucleotide, specific description: Methylated a protospacer adjacent motif CG non-target strand; (5Me˜dC represents a 5 methylated cytosine)
  source 1..40: mol_type = other DNA
  modified_base 34: mod_base = m5c

SEQ ID NO: 32 - DNA, 40 bp, synthetic construct; identical to SEQ ID NO: 28
  misc_feature 1..40: note = Description of sequence: Oligonucleotide, specific description: HEX-labelled a protospacer adjacent motif CG target strand
  source 1..40: mol_type = other DNA
  modified_base 1: mod_base = OTHER; note = nucleotide bound to Hexachlorofluorescein

SEQ ID NO: 33 - DNA, 40 bp, synthetic construct; identical to SEQ ID NO: 28
  misc_feature 1..40: note = Description of sequence: Oligonucleotide, specific description: HEX-labelled methylated a protospacer adjacent motif CG target strand
  source 1..40: mol_type = other DNA
  modified_base 1: mod_base = OTHER; note = nucleotide bound to Hexachlorofluorescein
  modified_base 6: mod_base = m5c

SEQ ID NO: 34 - DNA, 40 bp, synthetic construct; identical to SEQ ID NO: 27
  misc_feature 1..40: note = Description of sequence: Oligonucleotide, specific description: FAM-labelled a protospacer adjacent motif CC non-target strand
  source 1..40: mol_type = other DNA
  modified_base 1: mod_base = OTHER; note = 6-fluorescein amidite-d-thymine-puromycin

SEQ ID NO: 35 - DNA, 40 bp, synthetic construct; identical to SEQ ID NO: 29
  misc_feature 1..40: note = Description of sequence: Oligonucleotide, specific description: FAM-labelled a protospacer adjacent motif CG non-target strand
  source 1..40: mol_type = other DNA
  modified_base 1: mod_base = OTHER; note = 6-fluorescein amidite-d-thymine-puromycin

SEQ ID NO: 36 - DNA, 40 bp, synthetic construct; identical to SEQ ID NO: 29
  misc_feature 1..40: note = Description of sequence: Oligonucleotide, specific description: FAM-labelled methylated a protospacer adjacent motif CG non-target strand
  source 1..40: mol_type = other DNA
  modified_base 1: mod_base = OTHER; note = 6-fluorescein amidite-d-thymine-puromycin
  modified_base 34: mod_base = m5c

SEQ ID NO: 37 - DNA, 34 bp, synthetic construct
  misc_feature 1..34: note = Description of sequence: Oligonucleotide, specific description: K771A-F
  source 1..34: mol_type = other DNA
gaattttaacgcaaaccgggaagaatcgaatttg

SEQ ID NO: 38 - DNA, 18 bp, synthetic construct
  misc_feature 1..18: note = Description of sequence: Oligonucleotide, specific description: K711A-R
  source 1..18: mol_type = other DNA
caacggctgcgtaaatgg

SEQ ID NO: 39 - DNA, 22 bp, synthetic construct
  misc_feature 1..22: note = Description of sequence: Oligonucleotide, specific description: D723A-R
  source 1..22: mol_type = other DNA
atgcaaattcgattcttcccgg

SEQ ID NO: 40 - DNA, 22 bp, synthetic construct
  misc_feature 1..22: note = Description of sequence: Oligonucleotide, specific description: D1017A-F
  source 1..22: mol_type = other DNA
caaaccatcgcctcctccaatg

SEQ ID NO: 41 - DNA, 18 bp, synthetic construct
  misc_feature 1..18: note = Description of sequence: Oligonucleotide, specific description: D1017A-R
  source 1..18: mol_type = other DNA
ataataggcgaacagatc

SEQ ID NO: 42 - DNA, 21 bp, synthetic construct
  misc_feature 1..21: note = Description of sequence: Oligonucleotide, specific description: S1019A-F
  source 1..21: mol_type = other DNA
catcgactccgccaatggagg

SEQ ID NO: 43 - DNA, 23 bp, synthetic construct
  misc_feature 1..23: note = Description of sequence: Oligonucleotide, specific description: S1019A-R
  source 1..23: mol_type = other DNA
gtttgataataggcgaacagatc

SEQ ID NO: 44 - DNA, 27 bp, synthetic construct
  misc_feature 1..27: note = Description of sequence: Oligonucleotide, specific description: T9 gRNA target
  source 1..27: mol_type = other DNA
accgatcccctgagaggacagggaacc

SEQ ID NO: 45 - DNA, 27 bp, synthetic construct
  misc_feature 1..27: note = Description of sequence: Oligonucleotide, specific description: T9 gRNA reverse complement
  source 1..27: mol_type = other DNA
gacggttccctgtcctctcaggggatc

SEQ ID NO: 46 - DNA, 27 bp, synthetic construct
  misc_feature 1..27: note = Description of sequence: Oligonucleotide, specific description: T3 gRNA target
  source 1..27: mol_type = other DNA
accgagagacctgaacagcggagagtc

SEQ ID NO: 47 - DNA, 27 bp, synthetic construct
  misc_feature 1..27: note = Description of sequence: Oligonucleotide, specific description: T3 gRNA reverse complement
  source 1..27: mol_type = other DNA
gacgactctccgctgttcaggtctctc

SEQ ID NO: 48 - DNA, 27 bp, synthetic construct
  misc_feature 1..27: note = Description of sequence: Oligonucleotide, specific description: T4 gRNA target
  source 1..27: mol_type = other DNA
accgaatggagagggtcccggtgctgg

SEQ ID NO: 49 - DNA, 27 bp, synthetic construct
  misc_feature 1..27: note = Description of sequence: Oligonucleotide, specific description: T4 gRNA reverse complement
  source 1..27: mol_type = other DNA
gacccagcaccgggaccctctccattc

SEQ ID NO: 50 - DNA, 26 bp, synthetic construct
  misc_feature 1..26: note = Description of sequence: Oligonucleotide, specific description: T5 gRNA target
  source 1..26: mol_type = other DNA
accggagacagaggagaggccccgga

SEQ ID NO: 51 - DNA, 26 bp, synthetic construct
  misc_feature 1..26: note = Description of sequence: Oligonucleotide, specific description: T5 gRNA reverse complement
  source 1..26: mol_type = other DNA
gactccggggcctctcctctgtctcc

SEQ ID NO: 52 - protein, 1019 aa, synthetic construct
  REGION 1..1019: note = Description of sequence: Peptide, specific description: ThermoCas9
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASS

SEQ ID NO: 53 - DNA, 21 bp, synthetic construct
  misc_feature 1..21: note = Description of sequence: Oligonucleotide, specific description: D723A-R
  source 1..21: mol_type = other DNA
catgccgtcgctgctgccatc

SEQ ID NO: 54 - skipped

SEQ ID NO: 55 - DNA, 23 bp, synthetic construct
  misc_feature 1..23: note = Description of sequence: Oligonucleotide, specific description: EMX1 T4 Guide
  source 1..23: mol_type = other DNA
aatggagagggtcccggtgctgg

SEQ ID NO: 56-58 - skipped (3 entries)

SEQ ID NO: 59 - DNA, 23 bp, synthetic construct
  misc_feature 1..23: note = Description of sequence: Oligonucleotide, specific description: VEGFA T3 Guide
  source 1..23: mol_type = other DNA
agagacctgaacagcggagagtc

SEQ ID NO: 60-61 - skipped (2 entries)

SEQ ID NO: 62 - DNA, 23 bp, synthetic construct
  misc_feature 1..23: note = Description of sequence: Oligonucleotide, specific description: PRDX4 T5 (reverse complement) Guide
  source 1..23: mol_type = other DNA
ggagacagaggagaggccccgga

SEQ ID NO: 63-64 - skipped (2 entries)

SEQ ID NO: 65 - DNA, 39 bp, synthetic construct
  misc_feature 1..39: note = Description of sequence: Oligonucleotide, specific description: Nucleic Acid Sequence of Figure 3B
  source 1..39: mol_type = other DNA
ctagaggtaggatggcaagatcctggtatacaccaagct

SEQ ID NO: 66 - DNA, 39 bp, synthetic construct
  misc_feature 1..39: note = Description of sequence: Oligonucleotide, specific description: Nucleic Acid Sequence of Figure 3B
  source 1..39: mol_type = other DNA
gatctccatcctaccgttctaggaccatatctggttcga

SEQ ID NO: 67 - RNA, 125 nt, synthetic construct
  misc_feature 1..125: note = Description of sequence: RNA oligonucleotide, specific description: Secondary structures of the observed R-loop structure
  source 1..125: mol_type = other RNA
ggtattatggcaagatcctggtagtcatagttcccctggaaacagggttactatgataagggctttctgcctataggcagactgacccgtggcgttggggatcgcctattccccactctgggcgt

SEQ ID NO: 68 - protein, 23 aa, synthetic construct
  REGION 1..23: note = Description of sequence: Peptide, specific description: ThermoCas9 HNH Catalytic Site
TEVDHVIPYSRSLDENREKGNRT

SEQ ID NO: 69 - protein, 23 aa, synthetic construct
  REGION 1..23: note = Description of sequence: Peptide, specific description: AnCas9 HNH catalytic site
CQLDHIVPQAGPGSCNRSKSNTP

SEQ ID NO: 70 - protein, 23 aa, synthetic construct
  REGION 1..23: note = Description of sequence: Peptide, specific description: AceCas9 HNH Catalytic Site
SELDHIVPRTDGGSCNKEKGRRP

SEQ ID NO: 71 - protein, 23 aa, synthetic construct
  REGION 1..23: note = Description of sequence: Peptide, specific description: SpCas9 HNH Catalytic Site
YDVDHIVPQSFLKDKNRGKSDNV

SEQ ID NO: 72 - protein, 23 aa, synthetic construct
  REGION 1..23: note = Description of sequence: Peptide, specific description: CjCas9 HNH Catalytic Site
LEIDHIYPYSRSFDQNQEKLNQT

SEQ ID NO: 73 - protein, 23 aa, synthetic construct
  REGION 1..23: note = Description of sequence: Peptide, specific description: Nm1Cas9 HNH Catalytic Site
VEIDHALPFSRTWDENQNKGNQT

SEQ ID NO: 74 - protein, 23 aa, synthetic construct
  REGION 1..23: note = Description of sequence: Peptide, specific description: HpaCas9 HNH Catalytic Site
VEVDHALPFSRTWDENQNKGNLT

SEQ ID NO: 75 - protein, 23 aa, synthetic construct
  REGION 1..23: note = Description of sequence: Peptide, specific description: StCas9 HNH Catalytic Site
FEVDHILPLSITFDANQEKGQRT

SEQ ID NO: 76 - protein, 23 aa, synthetic construct
  REGION 1..23: note = Description of sequence: Peptide, specific description: SaCas9 HNH Catalytic Site
YEVDHIIPRSVSFDENSKKGNRT

SEQ ID NO: 77 - protein, 37 aa, synthetic construct
  REGION 1..37: note = Description of sequence: Peptide, specific description: ThermoCas9 RuvC catalytic site
MKYKIGLDIGITSHIELARKNREESNLHHAVDAAIVA

SEQ ID NO: 78 - protein, 33 aa, synthetic construct
  REGION 1..33: note = Description of sequence: Peptide, specific description: AnCas9 RuvC catalytic site
RVGIDVGTHSHVEHVRKDRIDRRHHAVDASVVA

SEQ ID NO: 79 - protein, 32 aa, synthetic construct
  REGION 1..32: note = Description of sequence: Peptide, specific description: AceCas9 RuvC catalytic site
RLGVDVGERSVVELARKRLDRRHHAVDAVVLT

SEQ ID NO: 80 - protein, 39 aa, synthetic construct
  REGION 1..39: note = Description of sequence: Peptide, specific description: SpCas9 RuvC catalytic site
MDKKYSIGLDIGTNSVIEMARKVREINNYHHAHDAYLNA

SEQ ID NO: 81 - protein, 36 aa, synthetic construct
  REGION 1..36: note = Description of sequence: Peptide, specific description: CjCas 9 RuvC catalytic site
MARILAFDIGISSNIELARKDRNNHLHHAIDAVIIA

SEQ ID NO: 82 - protein, 34 aa, synthetic construct
  REGION 1..34: note = Description of sequence: Peptide, specific description: Nm1Cas9 RuvC catalytic site
ILGLDIGIASHIETARKVRAENDRHHALDAVVVA

SEQ ID NO: 83 - protein, 39 aa, synthetic construct
  REGION 1..39: note = Description of sequence: Peptide, specific description: HpaCas9 RuvC catalytic site
KNLNYILGLDLGIASHIETGRKSREDNDRHHALDAVVVA

SEQ ID NO: 84 - protein, 37 aa, synthetic construct
  REGION 1..37: note = Description of sequence: Peptide, specific description: StCas9 RuvC catalytic site
MSDLVLGLDIGIGSVIEMARKTRDTYHHHAVDALIIA

SEQ ID NO: 85 - protein, 39 aa, synthetic construct
  REGION 1..39: note = Description of sequence: Peptide, specific description: SaCas9 RuvC catalytic site
MKRNYILGLDIGITSIIELARKERNKGYKHHAEDALIIA

SEQ ID NO: 86 - DNA, 75 bp, synthetic construct
  misc_feature 1..75: note = Description of sequence: Oligonucleotide, specific description: Indel 0 in Figure 9A
  source 1..75: mol_type = other DNA
cacccatcccctgagaggacagggaaccccatccaacagccagggggactcaccttcgtgatgattctgccctcc

SEQ ID NO: 87 - DNA, 74 bp, synthetic construct
  misc_feature 1..74: note = Description of sequence: Oligonucleotide, specific description: Indel -1 in Figure 9A
  source 1..74: mol_type = other DNA
cacccatcccctgagaggacagggaccccatccaacagccagggggactcaccttcgtgatgattctgccctcc

SEQ ID NO: 88 - DNA, 74 bp, synthetic construct
  misc_feature 1..74: note = Description of sequence: Oligonucleotide, specific description: Indel ‘in Figure 9A
  source 1..74: mol_type = other DNA
  variation 25: note = n is a, c, g or ,t
cacccatcccctgagaggacagggnaccccatccaacagccagggggactcaccttcgtgatgattctgccctc

SEQ ID NO: 89 - DNA, 73 bp, synthetic construct
  misc_feature 1..73: note = Description of sequence: Oligonucleotide, specific description: Indel -2 in Figure 9A
  source 1..73: mol_type = other DNA
cacccatcccctgagaggacaggaccccatccaacagccagggggactcaccttcgtgatgattctgccctcc

SEQ ID NO: 90 - DNA, 54 bp, synthetic construct
  misc_feature 1..54: note = Description of sequence: Oligonucleotide, specific description: Indel -21 in Figure 9A
  source 1..54: mol_type = other DNA
cacccatcccctgagaggacagggggactcaccttcgtgatgattctgccctcc

SEQ ID NO: 91 - DNA, 74 bp, synthetic construct; identical to SEQ ID NO: 87
  misc_feature 1..74: note = Description of sequence: Oligonucleotide, specific description: Indel -1 in Figure 9A
  source 1..74: mol_type = other DNA

SEQ ID NO: 92 - DNA, 58 bp, synthetic construct
  misc_feature 1..58: note = Description of sequence: Oligonucleotide, specific description: Indel -17 in Figure 9A
  source 1..58: mol_type = other DNA
cacccatcccctgagaggacagccagggggactcaccttcgtgatgattctgccctcc

SEQ ID NO: 93 - DNA, 65 bp, synthetic construct
  misc_feature 1..65: note = Description of sequence: Oligonucleotide, specific description: Indel -10 in Figure 9A
  source 1..65: mol_type = other DNA
cacccatcccctgagaggacagccaacagccagggggactcaccttcgtgatgattctgccctcc

SEQ ID NO: 94 - DNA, 68 bp, synthetic construct
  misc_feature 1..68: note = Description of sequence: Oligonucleotide, specific description: Indel -7 in Figure 9A
  source 1..68: mol_type = other DNA
cacccatcccctgagaggacagcatccaacagccagggggactcaccttcgtgatgattctgccctcc

SEQ ID NO: 95 - DNA, 54 bp, synthetic construct; identical to SEQ ID NO: 90
  misc_feature 1..54: note = Description of sequence: Oligonucleotide, specific description: Indel -21 in Figure 9A
  source 1..54: mol_type = other DNA

SEQ ID NO: 96 - DNA, 58 bp, synthetic construct
  misc_feature 1..58: note = Description of sequence: Oligonucleotide, specific description: Indel -17 in Figure 9A
  source 1..58: mol_type = other DNA
cacccatcccctgagaggacaggcagggggactcaccttcgtgatgattctgccctcc

SEQ ID NO: 97 - DNA, 65 bp, synthetic construct
  misc_feature 1..65: note = Description of sequence: Oligonucleotide, specific description: Indel -10 in Figure 9A
  source 1..65: mol_type = other DNA
cacccatcccctgagaggacaggcaacagccagggggactcaccttcgtgatgattctgccctcc

SEQ ID NO: 98 - DNA, 72 bp, synthetic construct
  misc_feature 1..72: note = Description of sequence: Oligonucleotide, specific description: Indel -3 in Figure 9A
  source 1..72: mol_type = other DNA
cacccatcccctgagaggacaggccccatccaacagccagggggactcaccttcgtgatgattctgccctcc

SEQ ID NO: 99 - DNA, 72 bp, synthetic construct
  misc_feature 1..72: note = Description of sequence: Oligonucleotide, specific description: Indel -2 in Figure 9A
  source 1..72: mol_type = other DNA
cacccatcccctgagaggacagggacccatccaacagccagggggactcaccttcgtgatgattctgcctcc

SEQ ID NO: 100 - DNA, 75 bp, synthetic construct; identical to SEQ ID NO: 86
  misc_feature 1..75: note = Description of sequence: Oligonucleotide, specific description: Indel 0 in Figure 9B
  source 1..75: mol_type = other DNA

SEQ ID NO: 101 - DNA, 74 bp, synthetic construct; identical to SEQ ID NO: 87
  misc_feature 1..74: note = Description of sequence: Oligonucleotide, specific description: Indel -1 in Figure 9B
  source 1..74: mol_type = other DNA

SEQ ID NO: 102 - DNA, 75 bp, synthetic construct
  misc_feature 1..75: note = Description of sequence: Oligonucleotide, specific description: Indel ‘in Figure 9B
  source 1..75: mol_type = other DNA
  variation 26: note = n is a, c, g or ,t
cacccatcccctgagaggacaggganaccccatccaacagccagggggactcaccttcgtgatgattctgccctc

SEQ ID NO: 103 - DNA, 58 bp, synthetic construct; identical to SEQ ID NO: 92
  misc_feature 1..58: note = Description of sequence: Oligonucleotide, specific description: Indel -17 in Figure 9B
  source 1..58: mol_type = other DNA

SEQ ID NO: 104 - DNA, 74 bp, synthetic construct; identical to SEQ ID NO: 87
  misc_feature 1..74: note = Description of sequence: Oligonucleotide, specific description: Indel -1 in Figure 9B
  source 1..74: mol_type = other DNA

SEQ ID NO: 105 - DNA, 73 bp, synthetic construct; identical to SEQ ID NO: 89
  misc_feature 1..73: note = Description of sequence: Oligonucleotide, specific description: Indel -2 in Figure 9B
  source 1..73: mol_type = other DNA

SEQ ID NO: 106 - DNA, 58 bp, synthetic construct; identical to SEQ ID NO: 92
  misc_feature 1..58: note = Description of sequence: Oligonucleotide, specific description: Indel -17 in Figure 9B
  source 1..58: mol_type = other DNA

SEQ ID NO: 107 - DNA, 65 bp, synthetic construct
  misc_feature 1..65: note = Description of sequence: Oligonucleotide, specific description: Indel -10 in Figure 9B
  source 1..65: mol_type = other DNA
cacccatcccctgagaggacatccaacagccagggggactcaccttcgtgatgattctgccctcc

SEQ ID NO: 108 - DNA, 65 bp, synthetic construct; identical to SEQ ID NO: 107
  misc_feature 1..65: note = Description of sequence: Oligonucleotide, specific description: Indel -10 in Figure 9B
  source 1..65: mol_type = other DNA

SEQ ID NO: 109 - DNA, 57 bp, synthetic construct
  misc_feature 1..57: note = Description of sequence: Oligonucleotide, specific description: Indel -18 in Figure 9B
  source 1..57: mol_type = other DNA
cacccatcccctgagaggacagcagggggactcaccttcgtgatgattctgccctcc

SEQ ID NO: 110 - DNA, 69 bp, synthetic construct
  misc_feature 1..69: note = Description of sequence: Oligonucleotide, specific description: Indel -6 in Figure 9B
  source 1..69: mol_type = other DNA
cacccatcccctgagaggacagccatccaacagccagggggactcaccttcgtgatgattctgccctcc

SEQ ID NO: 111 - DNA, 75 bp, synthetic construct; identical to SEQ ID NO: 86
  misc_feature 1..75: note = Description of sequence: Oligonucleotide, specific description: Indel 0 in Figure 9C
  source 1..75: mol_type = other DNA

SEQ ID NO: 112 - DNA, 74 bp, synthetic construct; identical to SEQ ID NO: 87
  misc_feature 1..74: note = Description of sequence: Oligonucleotide, specific description: Indel -1 in Figure 9C
  source 1..74: mol_type = other DNA

SEQ ID NO: 113 - DNA, 58 bp, synthetic construct; identical to SEQ ID NO: 96
  misc_feature 1..58: note = Description of sequence: Oligonucleotide, specific description: Indel -17 in Figure 9C
  source 1..58: mol_type = other DNA

SEQ ID NO: 114 - DNA, 74 bp, synthetic construct; identical to SEQ ID NO: 87
  misc_feature 1..74: note = Description of sequence: Oligonucleotide, specific description: Indel -1 in Figure 9C
  source 1..74: mol_type = other DNA

SEQ ID NO: 115 - DNA, 75 bp, synthetic construct; identical to SEQ ID NO: 102
  misc_feature 1..75: note = Description of sequence: Oligonucleotide, specific description: Indel ‘in Figure 9C
  source 1..75: mol_type = other DNA
  variation 26: note = n is a, c, g or ,t

SEQ ID NO: 116 - DNA, 58 bp, synthetic construct; identical to SEQ ID NO: 92
  misc_feature 1..58: note = Description of sequence: Oligonucleotide, specific description: Indel -17 in Figure 9C
  source 1..58: mol_type = other DNA

SEQ ID NO: 117 - DNA, 72 bp, synthetic construct; identical to SEQ ID NO: 98
  misc_feature 1..72: note = Description of sequence: Oligonucleotide, specific description: Indel -3 in Figure 9C
  source 1..72: mol_type = other DNA

SEQ ID NO: 118 - DNA, 54 bp, synthetic construct; identical to SEQ ID NO: 90
  misc_feature 1..54: note = Description of sequence: Oligonucleotide, specific description: Indel -21 in Figure 9C
  source 1..54: mol_type = other DNA

SEQ ID NO: 119 - DNA, 65 bp, synthetic construct; identical to SEQ ID NO: 93
  misc_feature 1..65: note = Description of sequence: Oligonucleotide, specific description: Indel -10 in Figure 9C
  source 1..65: mol_type = other DNA

SEQ ID NO: 120 - DNA, 68 bp, synthetic construct; identical to SEQ ID NO: 94
  misc_feature 1..68: note = Description of sequence: Oligonucleotide, specific description: Indel -7 in Figure 9C
  source 1..68: mol_type = other DNA

SEQ ID NO: 121 - DNA, 64 bp, synthetic construct
  misc_feature 1..64: note = Description of sequence: Oligonucleotide, specific description: Indel -10 in Figure 9C
  source 1..64: mol_type = other DNA
cacccatcccctgagaggacagcaacagccagggggactcaccttcgtgatgattctgccctcc

SEQ ID NO: 122 - DNA, 67 bp, synthetic construct
  misc_feature 1..67: note = Description of sequence: Oligonucleotide, specific description: Indel -7 in Figure 9C
  source 1..67: mol_type = other DNA
cacccatcccctgagaggacagatccaacagccagggggactcaccttcgtgatgattctgccctcc

SEQ ID NO: 123 - DNA, 75 bp, synthetic construct
  misc_feature 1..75: note = Description of sequence: Oligonucleotide, specific description: T3R1 T3R2 T3R3 Indel 0 in Figure 9D
  source 1..75: mol_type = other DNA
ctagcagagacctgaacagcggagagtcctcacgaaactgagggtgaacctcgtggtgcccagctctttctttct

SEQ ID NO: 124 - DNA, 75 bp, synthetic construct
  misc_feature 1..75: note = Description of sequence: Oligonucleotide, specific description: T4R1 Indel 0 in Figure 9D
  source 1..75: mol_type = other DNA
gtagaaatggagagggtcccggtgctgggcgccgaagaaggagtgcgggggctgcagcggggaggcgcccagctg

SEQ ID NO: 125 - DNA, 68 bp, synthetic construct
  misc_feature 1..68: note = Description of sequence: Oligonucleotide, specific description: T4R1 Indel -7 in Figure 9D
  source 1..68: mol_type = other DNA
gtagaaatggagagggtcccggtgccgaagaaggagtgcgggggctgcagcggggaggcgcccagctg

SEQ ID NO: 126 - DNA, 75 bp, synthetic construct
  misc_feature 1..75: note = Description of sequence: Oligonucleotide, specific description: T4R1 Indel -1 in Figure 9D
  source 1..75: mol_type = other DNA
  variation 26: note = n is a, c, g or ,t
gtagaaatggagagggtcccggtgcntgggcgccgaagaaggagtgcgggggctgcagcggggaggcgcccagct

SEQ ID NO: 127 - DNA, 68 bp, synthetic construct
  misc_feature 1..68: note = Description of sequence: Oligonucleotide, specific description: T4R1 Indel -7 in Figure 9D
  source 1..68: mol_type = other DNA
gtagaaatggagagggtcccggcgccgaagaaggagtgcgggggctgcagcggggaggcgcccagctg

SEQ ID NO: 128 - DNA, 68 bp, synthetic construct; identical to SEQ ID NO: 125
  misc_feature 1..68: note = Description of sequence: Oligonucleotide, specific description: T4R1 Indel -7 in Figure 9D
  source 1..68: mol_type = other DNA

SEQ ID NO: 129 - DNA, 75 bp, synthetic construct; identical to SEQ ID NO: 124
  misc_feature 1..75: note = Description of sequence: Oligonucleotide, specific description: T4R2 Indel 0 in Figure 9D
  source 1..75: mol_type = other DNA

SEQ ID NO: 130 - DNA, 68 bp, synthetic construct; identical to SEQ ID NO: 125
  misc_feature 1..68: note = Description of sequence: Oligonucleotide, specific description: T4R2 Indel -7 in Figure 9D
  source 1..68: mol_type = other DNA

SEQ ID NO: 131 - DNA, 74 bp, synthetic construct
  misc_feature 1..74: note = Description of sequence: Oligonucleotide, specific description: T4R2 Indel ‘in Figure 9D
  source 1..74: mol_type = other DNA
  variation 26: note = n is a, c, g or ,t
gtagaaatggagagggtcccggtgcntgggcgccgaagaggagtgcgggggctgcagcggggaggcgcccagct

SEQ ID NO: 132 - DNA, 68 bp, synthetic construct; identical to SEQ ID NO: 127
  misc_feature 1..68: note = Description of sequence: Oligonucleotide, specific description: T4R2 Indel -7 in Figure 9D
  source 1..68: mol_type = other DNA

SEQ ID NO: 133 - DNA, 75 bp, synthetic construct; identical to SEQ ID NO: 124
  misc_feature 1..75: note = Description of sequence: Oligonucleotide, specific description: T4R3 Indel 0 in Figure 9D
  source 1..75: mol_type = other DNA

SEQ ID NO: 134 - DNA, 46 bp, synthetic construct
  misc_feature 1..46: note = Description of sequence: Oligonucleotide, specific description: T4R3 Indel -7 in Figure 9D
  source 1..46: mol_type = other DNA
tgccgaagaaggagtgcgggggctgcagcggggaggcgcccagctg

SEQ ID NO: 135 - DNA, 53 bp, synthetic construct
  misc_feature 1..53: note = Description of sequence: Oligonucleotide, specific description: T4R3 Indel ‘in Figure 9D; N represents any nucleotide selected from adenine (A) thymine (T) guanine (G) or cytosine (C); R represents a purine nucleotide
  source 1..53: mol_type = other DNA
  variation 4: note = n is a, c, g or ,t
tgcntgggcgccgaagaaggagtgcgggggctgcagcggggaggcgcccagct

SEQ ID NO: 136 - DNA, 46 bp, synthetic construct
  misc_feature 1..46: note = Description of sequence: Oligonucleotide, specific description: T4R3 Indel -7 in Figure 9D
  source 1..46: mol_type = other DNA
cgccgaagaaggagtgcgggggctgcagcggggaggcgcccagctg

SEQ ID NO: 137 - DNA, 75 bp, synthetic construct
  misc_feature 1..75: note = Description of sequence: Oligonucleotide, specific description: T5R1 T5R2 T5R4 Indel 0 in Figure 9D
  source 1..75: mol_type = other DNA
ggctgggagacagaggagaggccccggactcgcgaagaggagtgccacttctacgcgggtggacaagtgtacccg

SEQ ID NO: 138 - DNA, 75 bp, synthetic construct; identical to SEQ ID NO: 86
  misc_feature 1..75: note = Description of sequence: Oligonucleotide, specific description: T9R1 T9R3 Indel 0 in Figure 10A
  source 1..75: mol_type = other DNA

SEQ ID NO: 139 - DNA, 74 bp, synthetic construct; identical to SEQ ID NO: 87
  misc_feature 1..74: note = Description of sequence: Oligonucleotide, specific description: T9R1 T9R3 Indel -1 in Figure 10A
  source 1..74: mol_type = other DNA

SEQ ID NO: 140 - DNA, 75 bp, synthetic construct; identical to SEQ ID NO: 102
  misc_feature 1..75: note = Description of sequence: Oligonucleotide, specific description: T9R1 T9R3 Indel ‘in Figure 10A
  source 1..75: mol_type = other DNA
  variation 26: note = n is a, c, g or ,t

SEQ ID NO: 141 - DNA, 75 bp, synthetic construct; identical to SEQ ID NO: 86
  misc_feature 1..75: note = Description of sequence: Oligonucleotide, specific description: t9r2 Indel 0 in Figure 10A
  source 1..75: mol_type = other DNA

SEQ ID NO: 142 - DNA, 74 bp, synthetic construct; identical to SEQ ID NO: 87
  misc_feature 1..74: note = Description of sequence: Oligonucleotide, specific description: t9r2 Indel -1 in Figure 10A
  source 1..74: mol_type = other DNA

SEQ ID NO: 143 - DNA, 75 bp, synthetic construct; identical to SEQ ID NO: 102
  misc_feature 1..75: note = Description of sequence: Oligonucleotide, specific description: t9r2 Indel ‘in Figure 10A
  source 1..75: mol_type = other DNA
  variation 26: note = n is a, c, g or ,t

SEQ ID NO: 144 - DNA, 67 bp, synthetic construct
  misc_feature 1..67: note = Description of sequence: Oligonucleotide, specific description: t9r2 Indel -9 in Figure 10A
  source 1..67: mol_type = other DNA
cacccatcccctgagaggacagggacaacagccagggggactcaccttcgtgatgattctgccctcc

SEQ ID NO: 145 - DNA, 68 bp, synthetic construct
  misc_feature 1..68: note = Description of sequence: Oligonucleotide, specific description: t9r2 Indel -7 in Figure 10A
  source 1..68: mol_type = other DNA
cacccatcccctgagaggacagggaccaacagccagggggactcaccttcgtgatgattctgccctcc

SEQ ID NO: 146 - DNA, 72 bp, synthetic construct
  misc_feature 1..72: note = Description of sequence: Oligonucleotide, specific description: t9r2 Indel -3 in Figure 10A
  source 1..72: mol_type = other DNA
cacccatcccctgagaggacagggaccatccaacagccagggggactcaccttcgtgatgattctgccctcc

SEQ ID NO: 147 - DNA, 58 bp, synthetic construct; identical to SEQ ID NO: 96
  misc_feature 1..58: note = Description of sequence: Oligonucleotide, specific description: t9r2 Indel -17 in Figure 10A
  source 1..58: mol_type = other DNA

SEQ ID NO: 148 - DNA, 75 bp, synthetic construct; identical to SEQ ID NO: 123
  misc_feature 1..75: note = Description of sequence: Oligonucleotide, specific description: T3R1 T3R2 T3R3 Indel 0 in Figure 10B
  source 1..75: mol_type = other DNA

SEQ ID NO: 149 - DNA, 74 bp, synthetic construct
  misc_feature 1..74: note = Description of sequence: Oligonucleotide, specific description: T4R1 T4R2 T4R3 Indel 0 in Figure 10B
  source 1..74: mol_type = other DNA
ctagaaatggagagggtcccggtctgggcgccgaagaaggagtgcgggggctgcagcggggaggcgcccagctg

SEQ ID NO: 150 - DNA, 75 bp, synthetic construct; identical to SEQ ID NO: 137
  misc_feature 1..75: note = Description of sequence: Oligonucleotide, specific description: T5R1 Indel 0 in Figure 10C
  source 1..75: mol_type = other DNA

SEQ ID NO: 151 - DNA, 71 bp, synthetic construct
  misc_feature 1..71: note = Description of sequence: Oligonucleotide, specific description: T5R1 Indel -4 in Figure 10C
  source 1..71: mol_type = other DNA
ggctgggagacagaggagaggccactcgcgaagaggagtgccacttctacgcgggtggacaagtgtacccg

SEQ ID NO: 152 - DNA, 61 bp, synthetic construct
  misc_feature 1..61: note = Description of sequence: Oligonucleotide, specific description: T5R1 Indel -14 in Figure 10C
  source 1..61: mol_type = other DNA
ggctgggagacagaggagaggccaggagtgccacttctacgcgggtggacaagtgtacccg

SEQ ID NO: 153 - DNA, 74 bp, synthetic construct
  misc_feature 1..74: note = Description of sequence: Oligonucleotide, specific description: T5R1 Indel -1 in Figure 10C
  source 1..74: mol_type = other DNA
ggctgggagacagaggagaggccccgactcgcgaagaggagtgccacttctacgcgggtggacaagtgtacccg

SEQ ID NO: 154 - DNA, 75 bp, synthetic construct
  misc_feature 1..75: note = Description of sequence: Oligonucleotide, specific description: T5R1 Indel ‘in Figure 10C; N represents any nucleotide selected from adenine (A) thymine (T) guanine (G) or cytosine (C); R represents a purine nucleotide
  source 1..75: mol_type = other DNA
  variation 26: note = n is a, c, g or ,t
ggctgggagacagaggagaggccccnggactcgcgaagaggagtgccacttctacgcgggtggacaagtgtaccc

SEQ ID NO: 155 - DNA, 62 bp, synthetic construct
  misc_feature 1..62: note = Description of sequence: Oligonucleotide, specific description: T5R1 Indel -13 in Figure 10C
  source 1..62: mol_type = other DNA
ggctgggagacagaggagaggccccggagtgccacttctacgcgggtggacaagtgtacccg

SEQ ID NO: 156 - DNA, 75 bp, synthetic construct; identical to SEQ ID NO: 137
  misc_feature 1..75: note = Description of sequence: Oligonucleotide, specific description: T5R2 Indel 0 in Figure 10D
  source 1..75: mol_type = other DNA

SEQ ID NO: 157 - DNA, 73 bp, synthetic construct
  misc_feature 1..73: note = Description of sequence: Oligonucleotide, specific description: T5R2 Indel -2 in Figure 10D
  source 1..73: mol_type = other DNA
ggctgggagacagaggagaggccccactcgcgaagaggagtgccacttctacgcgggtggacaagtgtacccg

SEQ ID NO: 158 - DNA, 64 bp, synthetic construct
  misc_feature 1..64: note = Description of sequence: Oligonucleotide, specific description: T5R2 Indel -11 in Figure 10D
  source 1..64: mol_type = other DNA
ggctgggagacagaggagaggccccgaggagtgccacttctacgcgggtggacaagtgtacccg

SEQ ID NO: 159 - DNA, 61 bp, synthetic construct
  misc_feature 1..61: note = Description of sequence: Oligonucleotide, specific description: T5R2 Indel -14 in Figure 10D
  source 1..61: mol_type = other DNA
ggctgggagacagaggagaggccccgagtgccacttctacgcgggtggacaagtgtacccg

SEQ ID NO: 160 - DNA, 75 bp, synthetic construct
  misc_feature 1..75: note = Description of sequence: Oligonucleotide, specific description: T5R2 Indel °in Figure 10D
  source 1..75: mol_type = other DNA
  variation 26..45: note = n is a, c, g or ,t
ggctgggagacagaggagaggccccnnnnnnnnnnnnnnnnnnnnggactcgcgaagaggagtgccacttctacg

SEQ ID NO: 161 - DNA, 73 bp, synthetic construct
  misc_feature 1..73: note = Description of sequence: Oligonucleotide, specific description: T5R2 Indel –in Figure 10D
  source 1..73: mol_type = other DNA
  variation 26..39: note = n is a, c, g or ,t
ggctgggagacagaggagaggccccnnnnnnnnnnnnnnactcgcgaagaggagtgccacttctacgcgggtg

SEQ ID NO: 162 - DNA, 75 bp, synthetic construct; identical to SEQ ID NO: 137
  misc_feature 1..75: note = Description of sequence: Oligonucleotide, specific description: T5R3 Indel 0 in Figure 10E
  source 1..75: mol_type = other DNA

SEQ ID NO: 163 - DNA, 74 bp, synthetic construct
  misc_feature 1..74: note = Description of sequence: Oligonucleotide, specific description: T5R3 Indel -1 in Figure 10E
  source 1..74: mol_type = other DNA
ggctgggagacagaggagaggcccggactcgcgaacaggagtgccacttctacgcgggtggacaagtgtacccg

SEQ ID NO: 164 - DNA, 74 bp, synthetic construct
  misc_feature 1..74: note = Description of sequence: Oligonucleotide, specific description: T5R3 Indel -1 in Figure 10E
  source 1..74: mol_type = other DNA
ggctgggagacagaggagaggccccgactcgcgaagaggagagccacttctacgcgggtggacaagtgtacccg

SEQ ID NO: 165 - DNA, 58 bp, synthetic construct
  misc_feature 1..58: note = Description of sequence: Oligonucleotide, specific description: T5R3 Indel -17 in Figure 10E
  source 1..58: mol_type = other DNA
ggctgggagacagaggagagaggagtgccacttctacgcgggtggacaagtgtacccg

SEQ ID NO: 166 - DNA, 55 bp, synthetic construct
  misc_feature 1..55: note = Description of sequence: Oligonucleotide, specific description: T5R3 Indel -17 in Figure 10E
  source 1..55: mol_type = other DNA
ggctgggagacagaggagaggagtgccacttctacgcggtggacaagtgtacccg

SEQ ID NO: 167 - DNA, 54 bp, synthetic construct
  misc_feature 1..54: note = Description of sequence: Oligonucleotide, specific description: T5R3 Indel -21 in Figure 10E
  source 1..54: mol_type = other DNA
ggctgggagacagaggagaggcgccacttctacgcgggtggacaagtgtacccg

SEQ ID NO: 168 - DNA, 69 bp, synthetic construct
  misc_feature 1..69: note = Description of sequence: Oligonucleotide, specific description: T5R3 Indel -6 in Figure 10E
  source 1..69: mol_type = other DNA
ggctgggagacagaggagaggcctcgcgaagaggagtgccacttctacgcgggtggacaagtgtacccg

SEQ ID NO: 169 - DNA, 58 bp, synthetic construct
  misc_feature 1..58: note = Description of sequence: Oligonucleotide, specific description: T5R3 Indel -12 in Figure 10E
  source 1..58: mol_type = other DNA
ggctgggagacagaggagaggggagtgccacttctacgcgggtggacaagtgtacccg

SEQ ID NO: 170 - DNA, 69 bp, synthetic construct; identical to SEQ ID NO: 168
  misc_feature 1..69: note = Description of sequence: Oligonucleotide, specific description: T5R3 Indel -6 in Figure 10E
  source 1..69: mol_type = other DNA

SEQ ID NO: 171 - DNA, 16 bp, synthetic construct
  misc_feature 1..16: note = Description of sequence: Oligonucleotide, specific description: Nucleic acid in Figure 12
  source 1..16: mol_type = other DNA
cctggtatataccagg

SEQ ID NO: 172-174 - skipped (3 entries)